(19) United States
(12) Patent Application Publication
(10) Pub. No.: US 2004/0247578 A1
(43) Pub. Date: Dec. 9, 2004

(54) Methods and reagents for inducing immunity

(75) Inventors: Michael T. Lotze, Pittsburgh, PA (US); Hideaki Tahara, Tokyo (JP)

Correspondence Address: TESTA, HURWITZ & THIBEAULT, LLP, HIGH STREET TOWER, 125 HIGH STREET, BOSTON, MA, 02110, US

(73) Assignee: University of Pittsburgh of Commonwealth System of Higher Education, Pittsburgh, PA

(21) Appl. No.: 10/688,845

(22) Filed: Oct. 15, 2003

Related U.S. Application Data
(60) Provisional application No. 60/418,865, filed on Oct. 15, 2002.

(51) Int. Cl. A61K 48/00; A61K 38/20

(52) U.S. Cl. 424/93.21; 514/44; 424/85.2

(57) ABSTRACT

Methods and reagents for treating tumors, metastases, and infectious lesions by co-administration of antigen presenting cells and immunostimulatory cytokines or nucleic acid encoding an immunostimulatory cytokine into or near the site of the tumor or infectious lesion are described.

### RELATED APPLICATIONS

This application claims priority to U.S. Application Ser. No. 60/418,865, filed Oct. 15, 2002, the entire disclosure of which is incorporated herein by reference.

### GOVERNMENT LICENSE RIGHTS

Certain work described herein was supported, in part, by Federal Grant No. PO1 CA59371, awarded by the National Cancer Institute of the National Institutes of Health. The Government may have certain rights in the invention.

### FIELD OF THE INVENTION

The invention is directed to the field of immunology. In particular, the invention is directed to the use of co-administration of antigen presenting cells and immunostimulatory cytokines to treat tumors or infections.

### BACKGROUND OF THE INVENTION

Antigen presenting cells (APCs), such as dendritic cells, perform a number of closely linked functions that result in the activation of T lymphocytes, including antigen processing, during which large antigen moieties, cells or proteins are degraded to fragments more recognizable by T cells. (Steinman (1991) Annu. Rev. Immunol. 9:271-296; Macatonia et al. (1989) J. Exp. Med. 169:1255-1264). For example, activated dendritic cells take up soluble antigen and apoptotic bodies, migrate to the paracortical T cell-rich areas of lymph nodes, and initiate a series of interactions leading to the selection of antigen-specific T cells and the release of the dendritic cell cytokines interferon-α (IFN-α) and interleukin-12 (IL-12).

Administration of dendritic cells pulsed with synthetic tumor-associated peptides have been used as therapeutic anti-tumor vaccines to induce an anti-tumor immune response in vitro and in mice following adoptive transfer (Mayordomo et al. (1995) Nature Med. 1: 1297-1302; Zitvogel et al. (1996) J. Exp. Med. 183:87-97; Porgador and Gilboa (1995) J. Exp. Med. 182:255-260; Porgador et al. (1996) J. Immunol. 156:2918-2926). However, tumor-associated antigens have been identified for only a limited number of human tumors. Several approaches to overcoming this problem, including pulsing dendritic cells with acid-eluted bulk tumor peptides (Zitvogel et al. (1996) supra), tumor extracts and RNA (Flamand et al. (1994) Eur. J. Immunol. 24:605-610; Ashley et al. (1997) J. Exp. Med. 186:1177-1182; Boczkowski et al. (1996) J. Exp. Med. 184:465-472), or fusing of tumor cells with dendritic cells (Gong et al. (1997) Nature Med. 3:558-561), have been employed in dendritic cell-based vaccination strategies against tumors. However, the preparation of clinical samples from human solid tumors is time consuming and problematic. For example, tumor samples are not always available or in an accessible location in the body for obtaining a surgical biopsy. In addition, the technical expertise for obtaining, maintaining and processing such tumor samples may not be available at certain medical centers. Further, handling of a tumor sample ex vivo may introduce contamination into the tumor sample. Still further, the full repertoire of natural effector cells, e.g., DCs, natural killer (NK) cells and T cells, and cytokines recruited from the host that participate in the activation of the DCs may not be available to the full extent or in the appropriate signaling sequence or concentration as is found in vivo and simulating such events ex vivo is cumbersome.

Immunostimulatory cytokines have also been used as therapeutics for eliciting or regulating an immune response. For example, interleukin 18 (IL-18) induces the synthesis of high levels of IFN-γ by natural killer (NK), T cells, B cells, and cells of the monocyte lineage (Okamura et al. (1995a) Nature 378: 88-91; Okamura et al. (1995b) Infect. Immun. 63:3966-72). IL-18 also plays an important role in T cell proliferation (Okamura et al. (1995a) supra), cytotoxic T lymphocyte (CTL) activation (Okamoto et al. (1999) J. Immunol. 162: 3202-11) and enhancement of NK cell activity (Okamura et al. (1995b) supra; Dao et al. (1998) J. Immunol. 161: 2217-22; Hashimoto et al. (1999) J. Immunol. 163: 583-9). IL-18 also induces Th2 cytokines, including IL-13, IL-4, and IL-10, in synergy with IL-2 (Hoshino et al. (1999) J. Immunol. 162: 5070-7; Hoshino et al. (2000) Eur. J. Immunol. 30: 1998-2006; Leite-De-Moraes et al. (2001) J. Immunol. 166: 945-51). Systemic administration of recombinant IL-18 (rIL-18) is associated with significant in vivo anti-tumor effects which appears to be mediated by NK cells as primary effector cells (Osaki et al. (1998) J. Immunol. 160: 1742-49; Micallef et al. (1997) Cancer Immunol. Immunother. 43: 361-67). Further, established subcutaneous tumors can be successfully treated by intratumoral (i.t.) injection of recombinant adenoviral vectors expressing biologically active mIL-18 (Ad.PTH.IL-18) (Osaki et al. (1999) Gene Ther. 6: 808-15). However, administration of such immunostimulatory cytokines alone result in only a modest biological response and high dosages are often needed to produce an effect in an animal, which may be toxic or produce deleterious side effects. A need therefore remains for more effective and safe therapies that can elicit a strong immune response against specific tumor-associated antigens or pathogens.

### SUMMARY OF THE INVENTION

The instant invention relates to methods and reagents for treating a tumor or infection by administering an immunostimulatory cytokine, or a nucleic acid encoding an immunostimulatory cytokine, in combination with antigen presenting cells into or near a tumor or infectious lesion. In a preferred embodiment, the invention relates to the anti-tumor effects associated with intratumor (i.t.) injection of an IL-18-expressing adenovirus in combination with dendritic cells. Intratumor injection of a combination of IL-18-expressing adenovirus and dendritic cells was associated with potent anti-tumor effects not only on the treated tumor lesion but also on distant lesions (e.g., metastases).

In one aspect, the invention provides methods for preventing or treating a primary tumor. In another aspect, the invention provides methods for preventing or treating a metastasis. In yet another aspect, the invention provides methods for preventing or treating an infection. The method comprises administering to a subject an effective amount of an antigen presenting cell and an immunostimulatory cytokine or a nucleic acid encoding an immunostimulatory cytokine. In an embodiment, the nucleic acid is operatively linked to an expression vector. The methods of the invention can be used to stop tumor growth or to reduce tumor size. The methods of the invention can also be used to inhibit or neutralize an infectious pathogen. In a preferred embodiment, the tumor or infectious lesion is injected, and the size of the tumor or infectious lesion, or other indicia of the tumor or infectious lesion, is monitored. In another embodiment, a subject is injected with antigen presenting cells and a cytokine or cytokine nucleic acid at a site adjacent the tumor or infectious lesion, e.g., within the same organ site of the tumor or infectious lesion.

In still another aspect, the invention provides reagents for inhibiting or treating tumor growth, metastasis, or infection. A therapeutic composition comprising an antigen presenting cell combined with an immunostimulatory cytokine or a nucleic acid encoding an immunostimulatory cytokine is provided. The antigen presenting cell and immunostimulatory cytokine or nucleic acid encoding an immunostimulatory cytokine may be administered together as an admixture or may be administered separately or sequentially.

In an embodiment, the antigen presenting cell is a dendritic cell. Exemplary dendritic cells are CD34+-derived dendritic cells, bone marrow-derived dendritic cells, monocyte-derived dendritic cells, splenocyte derived dendritic cells, skin-derived dendritic cells, follicular dendritic cells, and germinal center dendritic cells, for example. The dendritic cell may be, for example, a CD34+-derived dendritic cell cultured in the presence of granulocyte colony stimulating factor, granulocyte macrophage colony stimulatory factor, tumor necrosis factor α, interleukin 4, the Flt-3 ligand, and/or the kit ligand. In another embodiment, the antigen presenting cell is a Langherhans' cell, an interdigitating cell, a B cell, or a macrophage, or other cell that presents antigen and synthesizes the appropriate co-stimulatory molecules (e.g., cytokines).

In an embodiment, the immunostimulatory cytokine is interleukin-1α (IL-1α or IL-1f1), interleukin-1β (IL-1β or IL-1f2), interleukin-2 (IL-2), interleukin-3 (IL-3), interleukin-4 (IL-4), interleukin-6 (IL-6), interleukin-8 (IL-8), interleukin-9 (IL-9), interleukin-10 (IL-10), interleukin-12 (IL-12), other members of the IL-12 family, (e.g., IL-23, and IL-27), interleukin-18 (IL-18 or IL-1f4), interleukin-19 (IL-19), interleukin-20 (IL-20), other IL-1 family members, such as IL-1f3, IL-1 f5, IL-1 f6, IL-1f7, IL-1f8, IL-1f9, and IL-1f10; interferon-α (INF-α), interferon-β (IFN-β), interferon-γ (IFN-γ), tumor necrosis factor α (TNFα), transforming growth factor-β (TGFβ), granulocyte colony stimulating factor (GCSF), macrophage colony stimulating factor (MCSF), granulocyte-macrophage colony stimulating factor (GMCSF), the Flt-3 ligand, or the kit ligand, for example. In a preferred embodiment, the cytokine is interleukin-18, however any factor or cytokine made by a cell acting on another cell to elicit an immune response may be used.

In an embodiment, the expression vector used in the methods of the invention is a viral vector, such as, for example, an adenoviral vector, an adeno-associated viral vector, a retroviral vector, a herpes virus, a vaccinia virus, lentivirus, etc., or other exogenous plasmid.

The methods of the invention may be used to treat melanoma, hepatoma, adenocarcinoma, colorectal cancer, basal cell cancer, oral cancer, nasopharyngeal cancer, laryngeal cancer, bladder cancer, head and neck cancer, renal cell cancer, pancreatic cancer, pulmonary cancer, cervical cancer, ovarian cancer, esophageal cancer, gastric cancer, prostate cancer, testicular cancer, or breast cancer, for example.

The methods of the invention may also be used to treat an infection caused by a pathogen such as a virus, bacteria, parasite, prion, yeast, or fungus, for example.

### BRIEF DESCRIPTION OF THE DRAWINGS

The foregoing and other objects, features and advantages of the present invention, as well as the invention itself, will be more fully understood from the following description of preferred embodiments when read together with the accompanying drawings, in which:

FIG. 1 shows that intratumoral (i.t.) injection of Ad.PTH.IL-18 inhibited the growth of MCA205 tumors in wild type (WT) mice, but not in gld mice, and in turn enhanced CTL activity. FIG. 1 (A and B) shows the anti-tumor effects of Ad.PTH.IL-18 injection alone. WT mice and gld mice (five mice per group) received 1×10⁵ of MCA205 cells i.d. inoculation in the right flank on day 0. The animals were treated by i.t. injection of HBSS, Ad.EGFP, or Ad.PTH.IL-18 on day 7 and day 10. Data represent the mean±S.D. of tumor area. The i.t. injection of Ad.PTH.IL-18 was associated with a significant anti-tumor effect in WT animals (p=0.003; WT/Ad.PTH.IL-18 vs. WT/Ad.EGFP,p=0.003; WT/Ad.PTH.IL-18 vs WT/HBSS) (A), but not in gld mice (>0.05; gld/Ad.PTH.IL-18 vs gld/Ad.EGFP or gld/HBSS) (B). **;p<0.01, n.s.; not significant (C) CTL activities after treatment; Data represent the mean±S.D. of cytotoxic activity in the duplicate culture cells from regional lymph nodes cells obtained from two mice in each group. On day 7 and day 10 Ad.PTH.IL-18, Ad.EGFP or HBSS was administered, two in each group were sacrificed for the in vitro assay on day 14. After 5 days co-cultured with irradiated MCA205 cells, cytotoxic activity was assessed against MCA205 cells (A) and YAC-1 cells (B). There is a statistically significant difference between WT/Ad.PTH.IL-18 vs WT/Ad.EGFP (p=0.013) and WT/Ad.PTH.IL-18 vs gld/Ad.PTH.IL-18 (p=0.021). n.s., not significant.

FIG. 2 shows that coinjection of Ad.PTH.IL-18 and DC induced potent anti-tumor effect against not only injected MCA205 tumor but also non-injected tumor at the distant site. On day 0, mice received 1×10⁵ MCA205 cells on both the right and left flanks. On day 7 and 10, mice received 1×10⁶ DC and 3×10⁸ plaque forming units (p.fu.) of adenoviral vector, or HBSS. Data represent the mean±S.D. of tumor area of (A) injected and (B) non-injected tumor area of the animals. (C) Data represent the mean±S.D. of cytolytic activity of the cells cultured from regional lymph nodes or spleen harvested from treated animals. On day 7 and day 10, Ad.PTH.IL-18, Ad.EGFP or HBSS and DC were administered; two in each group were sacrificed for the in vitro assay on day 14. After 5 days of co-culture with irradiated MCA205 cells, cytotoxic activity was assessed against MCA205 cells and YAC-1 cells.

FIG. 3 shows that CTL induced by i.t. coinjection of Ad.PTH.IL-18 and DC are tumor specific and MHC class I restricted. In FIG. 3(A), cytolytic activity was assessed against MCA205 cells, MC38 cells, EL-4 cells, B16 cells, and YAC-1 cells at various effector: target (E:T) ratios. Data represent the mean±S.D. of cytotoxicity. There is a significant difference between cytolytic activity against MCA205 cells and those against other targets (p<0.01 for all). In FIG. 3(B), effector cells were treated with anti-H2K^{b} antibody, anti-H2K^{d} antibody, or no antibody at various E:T ratios. Data represent the mean±S.D. of cytotoxicity. There was a statistically significant difference between cytotoxicity of the anti-H2K^{b} treated group and that of the non-treated or anti-H2K^{d} treated group (*: p<0.05).

FIG. 4 shows the involvement of endogenous IL-12 in the anti-tumor effects mediated by AD.PTH-IL-18 and DCs using DCs cultured from IL-12 gene deficient (IL-12 GKO) mice. Coinjection of Ad.PTH.IL-18 and DC from IL-12 GKO mice was associated with significantly less anti-tumor effects when compared with that of the treatment with DCs from immunocompetent animals.

FIG. 5 shows a table of IL-1 homologs.

FIG. 6 shows an alignment of IL-1 homologs.

### DETAILED DESCRIPTION OF THE INVENTION

The present invention relates to the discovery that co-administration of antigen-presenting cells (APCs) with an immunostimulatory cytokine, or nucleic acid encoding an immunostimulatory cytokine, into or near a tumor or infectious lesion induces a specific immunological response against antigens associated with the tumor or infectious lesion without pre-loading or pulsing the APCs with the antigens. For example, APCs such as dendritic cells (DCs) may be co-injected with IL-18 or an expression vector expressing IL-18, into or near the site of a tumor or infectious lesion to induce a specific immune response against antigens associated with the tumor or infectious lesion. Injection of dendritic cells and IL-18 can cause tumor regression both at the site of injection and at distant sites, such as metastases.

The below examples demonstrate the in vivo anti-tumor effects of intratumoral (i.t.) administration of an adenoviral vector expressing functional mouse interleukin-18 (Ad.PTH.IL-18) using murine tumor systems. Potent anti-tumor effects were observed when an established MCA205 fibrosarcoma was treated in syngeneic immunocompetent mice with i.t. injection of Ad.PTH.IL-18 (p=0.0025 vs. control vector treatment), and potent cytotoxic T lymphocytes (CTLs) were generated in culture. In contrast, this in vivo anti-tumor effect was absent, and cytotoxic activity was significantly less (p=0.021) in Fas ligand-deficient gld mice. Co-injection of an MCA205 fibrosarcoma and an MC38 adenocarcinoma with both Ad.PTH.IL-18 and dendritic cells inhibited growth of the injected tumors. Further, an anti-tumor effect was also observed on a distant tumor inoculated intradermally (i.d.) in the contralateral flank of a mouse. In vitro analysis showed that the induced cytolytic activity was tumor-specific and MHC class I restricted. These studies suggest that cancer patients with multiple distant metastasis could be treated using this strategy.

Choice and Isolation of Antigen Presenting Cell

The present invention employs antigen presenting cells preferably professional antigen presenting cells, more preferably, dendritic cells CD(s), particularly CD34+-derived DCs (CD34+-DCs) harvested from mobilized peripheral blood, and bone marrow-derived dendritic cells (BM-DCs), harvested from bone marrow. Other DCs that may be useful in the invention include monocyte-derived DCs harvested from blood, CD34+-DCs harvested from bone marrow, splenocyte derived DCs harvested from the spleen, skin-derived DCs, follicular dendritic cells, and germinal center dendritic cells. Methods of isolating these dendritic cells from the tissues in which they arise and/or localize are well known in the art. For example, methods for isolating BM-DCs are described in Inaba et al. ((1992), J. Exp. Med. 176:1693-1702). Alternatively, CD34+progenitor cells may be obtained from human umbilical cord or adult blood, and may be stimulated with cytokines to differentiate into dendritic cells (see, e.g., Caux et al. (1996) J. Exp. Med. 184:695-706; Romani et al. (1994) J. Exp. Med. 180:83-93). The effectiveness of the Flt-3 ligand in generating dendritic cells is described in, e.g., Shurin et al. (1997) Cell Immunol. 179:174-184. BM-DCs or CD34+-DCs cultured with GM-CSF and IL-4 for several (e.g., 5 days) are particularly preferred. TNF-α and the kit ligand are also effective at increasing the yield of DCs grown in culture (see, e.g., Mayordomo et al. (1997) Stem Cells 15:94-103, and references cited therein), and may be used to obtain the DCs of the present invention. A majority of such DCs may display the immature phenotype as determined by flow cytometry and mixed lymphocyte reaction assay in accordance with previous reports (Pierre et al. (1997), Nature 388:787-792; Inaba et al. (1993), J. Exp. Med. 178:479-488).

Preferably, the original source of the APCs is the subject to be treated, such that the APCs are autologous. Allogeneic APCs, obtained from other individuals, may also be employed in the present invention, but preferably the APCs are derived from histocompatible or syngeneic individuals so as to provide proper MHC presentation to the cognate, antigen-specific T cell receptors of the subject. In addition, genetically engineered animals, such as mice or pigs, may be created which express human or humanized MHC proteins, and optionally co-stimulatory molecules, and may be used as a renewable source of APCs capable of proper MHC presentation to the cognate, antigen-specific T cell receptors of the subject.

The number of antigen presenting cells to be introduced into a subject depends upon a number of factors, including the number of sites at which the cells are to be administered (e.g., injected), the number of administrations that are to be performed over time, the size of the tumor or infectious lesion, and the nature of the tumor or infectious lesion. Although the number of cells to be used will vary with such factors, it is presently expected that up to about 109, preferably about 104 to about 108, more preferably about 105 to about 107, cells are administered per site, per treatment.

The antigen presenting cells may be clonally expanded prior to administration by standard techniques of cell culture that are well known in the art. If the antigen presenting cells are autologous to the subject, the steps of obtaining and, optionally, clonally expanding the antigen presenting cells is preferably performed as closely as possible to the time of administration. If, however, heterologous but syngeneic antigen presenting cells are used, the cells may be obtained and, optionally, clonally expanded far in advance of administration, and may be maintained indefinitely prior to use.

Choice of Cytokine

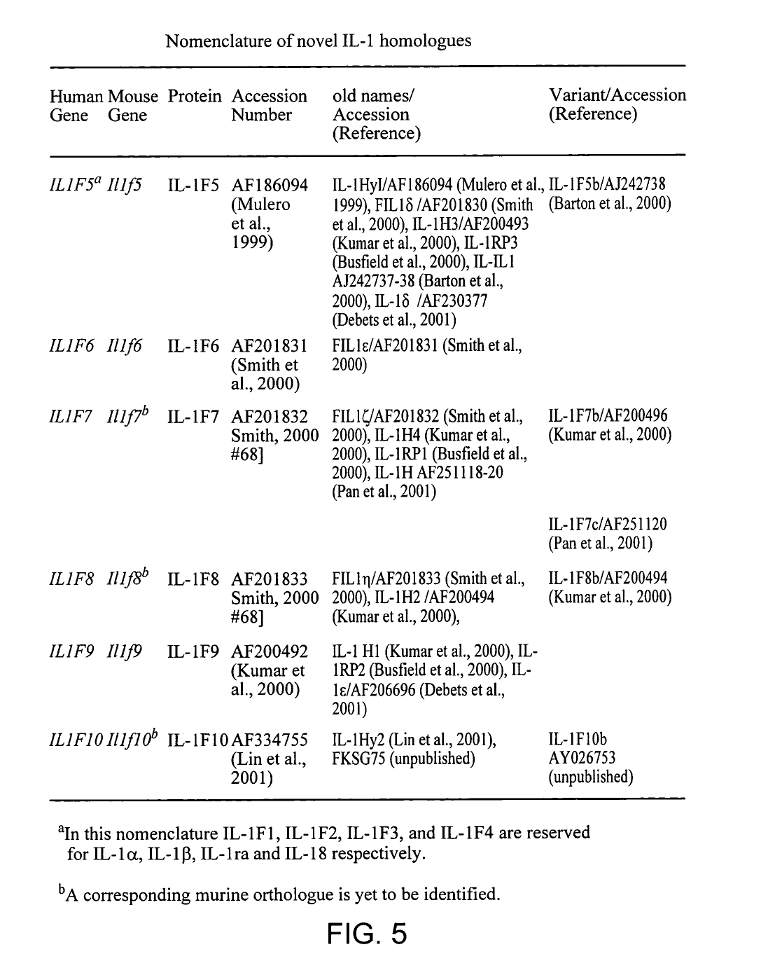

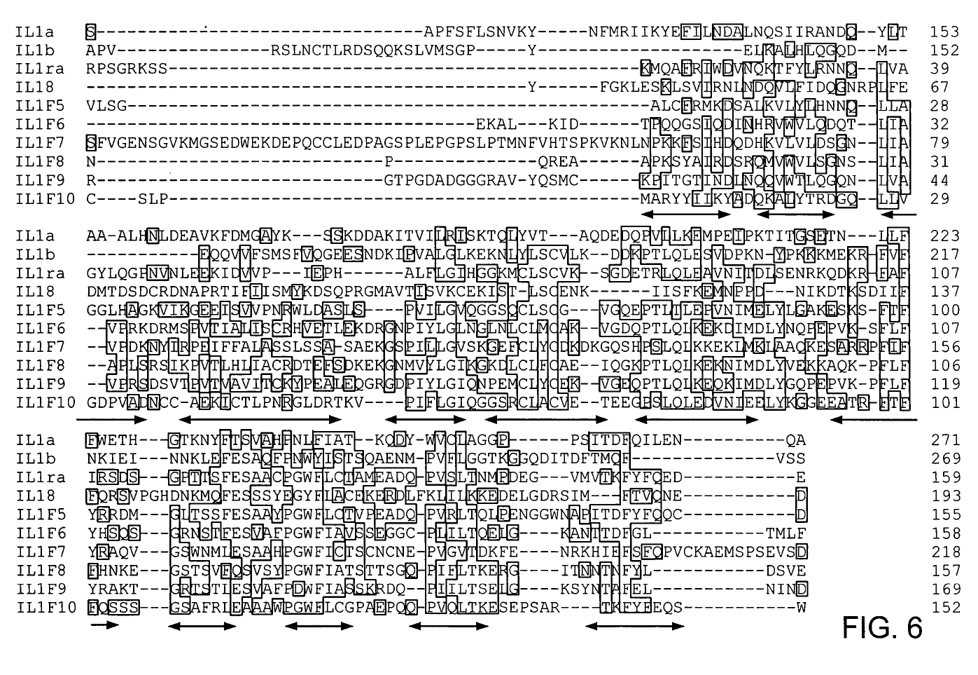

The immunostimulatory cytokine of the invention is a soluble molecule that mediates interactions among immune system cells or that causes an activation/increase or deactivation/decrease in an immune response against an antigenic peptide presented by an APC. Immunostimulatory cytokines include, but are not limited to, interleukin-1α (IL-1α), interleukin-1β (IL-1β), interleukin-2 (IL-2), interleukin-3 (IL-3), interleukin-4 (IL-4), interleukin-6 (IL-6), interleukin-8 (IL-8), interleukin-9 (IL-9), interleukin-10 (IL-10), interleukin-12 (IL-12), interleukin-18 (IL-18), interleukin-19 (IL-19), interleukin-20 (IL-20), ), interleukin-23 (IL-23), interleukin-27 (IL-27), IL-1f3, IL-1f5, IL-1f6, IL-1f7, IL-1f8, IL-1f9, and IL-1f10, interferon-α (IFN-α), interferon-β (IFN-β), interferon-γ (IFN-γ), tumor necrosis factor α (TNF-α), transforming growth factor-β (TGF-β), granulocyte colony stimulating factor (GCSF), macrophage colony stimulating factor (M-CSF), granulocyte-macrophage colony stimulating factor (GM-CSF), the Flt-3 ligand, and the kit ligand. The sequences for these immunostimulatory cytokines are known in the art and can be found, e.g,. in Genbank ((http://www.ncbi.nlm.nih.gov/), e.g., a Genbank accession number for IL-1a is NM000575; a Genbank accession number for IL-1 is X65019; a Genbank accession number for IL-2 is AF359939; a Genbank accession number for IL-3 is NM000588; a Genbank accession number for IL-4 is NM000589; a Genbank accession number for IL-6 is NM000600; a Genbank accession number for IL-8 is M2130; a Genbank accession number for IL-9 is M86593; a Genbank accession number for IL-10 is AF295024; a Genbank accession number for IL-12 is AF101062; a Genbank accession number for IL-18 is E17135; a Genbank accession number for IL-19 is NM013371; a Genbank accession number for IL-20 is AF212311; a Genbank accession number for IL-23 is NM016584; a Genbank accession number for IL-27 is AY099296; a Genbank accession number for IFN-α is M28586; a Genbank accession number for IFN-β is M28622; a Genbank accession number for IFN-γ is U10360; a Genbank accession number for TNF-α is NM000594; a Genbank accession number for TGF-β is AY149344; a Genbank accession number for GCSF Ml 7706; a Genbank accession number for M-CSF is M27087; a Genbank accession number for GM-CSF is M11734; a Genbank accession number for Flt-3 ligand is U03858; and a Genbank accession number for kit ligand is NM003994. See also Sims et al. (2001) Trends Immunol. 22:536, and sequences cited therein. See also FIGS. 5 and 6.

References to any of these cytokines are intended to embrace human homologs and variants and any other mammalian homologs or variants having activity in humans that is substantially similar to the human protein. The amino acid sequences of these cytokines are well known in the art. An amino acid sequence of IL-4 may be found in, for example, Arai et al. (1989) J. Immunol. 142(1):274-282; an amino acid sequence of interleukin-6 may be found in, for example, Yasukawa et al. (1987) EMBO J. 6(10):2939-2945; amino acid sequences of the p35 and p40 subunits of IL-12 may be found in, for example, Wolf et al. (1991) J. Immunol. 146(9):3074-3081; amino acid sequences of various IFN-α subtypes may be found in, for example, Gren et al. (1984) J. Interferon Res. 4(4):609-617, and Weismann et al. (1982) Princess Takamatsu Symp. 12:1-22; an amino acid sequence of TNF may be found in, for example, Pennica et al. (1984) Nature 312:724-729; an amino acid sequence of G-CSF may be found in, for example, Hirano et al. (1986) Nature 324:73-76; and an amino acid sequence of GM-CSF may be found in, for example, Cantrell et al. (1985) Proc. Natl. Acad. Sci. (USA) 82(18):6250-6254.

One of ordinary skill in the art may choose to use a vector comprising an isolated naturally occurring nucleic acid sequence that encodes the immunostimulatory cytokine (e.g., a genomic DNA, cDNA or RNA sequence) or may, utilizing the degeneracy of the genetic code, design and produce a vector comprising a non-naturally occurring sequence that still encodes a functional cytokine. In addition, a cytokine having amino acid sequence variations that do not adversely alter the activity or function of the cytokine may also be useful in the invention. For example, variations in the amino acid sequence of a cytokine that do not alter the residues that are conserved among humans and other mammals may be used. In the case of heterodimeric immunostimulatory cytokines (e.g., IL-12), nucleic acids that encode both subunits of the cytokine molecule must be either present in the expression vector or present on different expression vectors and co-administered. The expression vectors may contain any sequences know in the art required for expression of the operatively linked cytokine nucleic acid sequence, such as promoters, enhancers, etc.

In one embodiment of the invention, the cytokine nucleic acids are linked to an appropriate replicative cloning vector, a number of which are well known in the art. For expression, the subject nucleic acids can be operably linked to a transcriptional regulatory sequence, e.g., at least one of a transcriptional promoter (e.g., for constitutive expression or inducible expression) or transcriptional enhancer sequence. Such regulatory sequences in conjunction with a cytokine nucleic acid molecule can provide a useful vector for gene expression. This invention also comtemplates the use of host cells transfected with the cytokine expression vector.

Any vector capable of transporting and expressing the cytokine nucleic acid may be used. One type of vector is an episome, i.e., a nucleic acid capable of extra-chromosomal replication. Preferred vectors are those capable of autonomous replication and/or expression of nucleic acids to which they are linked. In general, expression vectors of utility in recombinant DNA techniques are often in the form of “plasmids” which refer generally to circular double stranded DNA loops which, in their vector form are not bound to the chromosome. However, the invention is intended to include such other forms of expression vectors, including RNA vectors, which serve equivalent functions and which become known in the art subsequently hereto.

However, it may be difficult to achieve expression of sufficient intracellular concentrations of the cytokine in certain instances. Therefore a preferred approach utilizes a recombinant DNA construct in which the cytokine nucleic acid is placed under the control of a strong pol III or pol II promoter. The use of such a construct results in the transcription of sufficient amounts of cytokine mRNA. For example, a vector can be introduced in vivo such that it is taken up by a cell and directs the transcription of an cytokine RNA. Such a vector can remain episomal or become chromosomally integrated, as long as it can be transcribed to produce the desired cytokine mRNA. Such vectors can be constructed by recombinant DNA technology methods standard in the art. Vectors can be plasmid, viral, or others known in the art, used for replication and expression in mammalian cells. Expression of the sequence encoding the cytokine mRNA can be by any promoter known in the art to act in mammalian, preferably human cells. Such promoters can be inducible or constitutive and can include but not be limited to: the SV40 early promoter region, the promoter contained in the 3′ long terminal repeat of Rous sarcoma virus, the herpes thymidine kinase promoter, the regulatory sequences of the metallothionein gene, etc. Any type of plasmid, cosmid, YAC or viral vector can be used to prepare the cytokine expression vector that can be introduced directly into the tissue site. Alternatively, viral vectors can be used which selectively infect the desired tissue, in which case administration may be accomplished by another route (e.g., systematically).

Variants of the immunostimulatory cytokines may also be administered. For example, for those cytokines having both pro-forms and mature forms (e.g., before and after cleavage of a signal peptide, or before and after limited proteolysis to yield an active fragment), either the pro- or mature form may be administered with the antigen presenting cells. Other variants, such as fusion proteins between an active fragment of a cytokine and a heterologous sequence (e.g., a heterologous signal peptide), may also be administered. Species variants may also be employed to the extent that they retain activity in a human subject. Thus, for example, murine, bovine, equine, ovine, feline, canine, non-human primate or other mammalian variant of a human cytokine may be used in the methods of the invention if these species variants retain activity that is substantially similar to their human homologs.

“Homology” or “identity” or “similarity” refers to sequence similarity between two peptides or between two nucleic acid molecules and can be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. An “unrelated” or “non-homologous” sequence shares less than about 40% identity, though preferably less than about 25% identity. Percent identity, homology or similarity are determined using a sequence alignment software such as BLAST. Alternatively percent identity, homology or similarity are determined by the number of nucleotide or amino acid differences in a sequence of a certain length. For example, a 100 residue sequence with 20 residue differences is defined as 80% identical, wherein a difference means a different residue or lack of residue. “Homologous” refers to the evolutionary relatedness of two nucleic acid or protein sequences. “Identity” refers to the degree to which nucleic acids or amino acids are the same between two sequences. “Similarity” refers to the degree to which nucleic acids or amino acids are the same, but includes neutral amino acid substitutions that do not significantly change the function of the protein as is well known in the art. Similarity also refers to neutral degenerate nucleic acids that may be substituted within a codon without changing the amino acid identity of the codon, as is well known in the art.

Subjects for treatment with the methods of the present invention include cancer patients and patients having an infection. Because it is preferred that the APCs and immunostimulatory cytokines or immunostimulatory cytokine nucleic acids can be administered directly into or near (e.g., within the same organ site(s)) of a tumor or infectious lesion, the present methods are useful in subjects having at least one physically well-defined tumor or infectious lesion. In addition, administration of the APCs and cytokines of the invention into one site at which a tumor or infectious lesion is present can lead to the development of a systemic immune response. The methods of the invention are therefore effective in treating diffuse or highly metastasized cancers or secondary infections if at least one site of a tumor or infectious lesion can be identified at which the APCs and immunostimulatory cytokine or immunostimulatory cytokine nucleic acid can be administered and can effectively load tumor or pathogen-associated antigen(s).

The methods of the invention may be used to treat patients that have solid tumors, into which the APCs and immunostimulatory cytokines or immunostimulatory cytokine expression vectors of the invention may be directly administered (e.g., injected). Appropriate solid tumors include melanomas, hepatomas, colorectal cancers, adenocarcinomas, basal cell cancers, oral cancers, nasopharyngeal cancers, laryngeal cancers, bladder cancers, head and neck cancers, renal cell cancers, pancreatic cancers, pulmonary cancers, cervical cancers, ovarian cancers, esophageal cancers, gastric cancers, prostate cancers, testicular cancers, and breast cancers, for example. For many of these cancers, an association between DC infiltration and prognosis has been established.

The methods of the invention may be used to treat patients that have an infectious lesion, to which the APCs and immunostimulatory cytokines or immunostimulatory cytokine nucleic acids of the invention may be directly administered. The infectious lesion may be caused by a pathogen such as a virus, bacteria, parasite, prion, fungus, or yeast, for example.

The antigen presenting cells and immunostimulatory cytokines of the invention may be administered by any of a number of methods. In a preferred embodiment, the APCs and immunostimulatory cytokines or immunostimulatory cytokine nucleic acids are injected using standard sterile techniques for intratumoral, subcutaneous, intradermal, transdermal, intramuscular, intraperitoneal, or other forms of injection.

The cells and cytokines or expression vectors may be administered together as a combined therapeutic or may be administered separately or sequentially. The cells and cytokines may be administered in a physiologically acceptable solution or buffer and may be administered in combination with other agents such as proteins, peptides, small molecules, antibodies, antibody fragments, or lipids, particularly cytokines such as G-CSF or IL-12, which may promote the ability of the antigen presenting cells to survive, load antigen, traffic to the draining lymph nodes or spleen, and present antigen to activate an immune response.

It is contemplated that the antigen presenting cells and immunostimulatory cytokines or cytokine nucleic acids encoding an immunostimulatory cytokine may be incorporated into any suitable carrier prior to use. More specifically, the dose, mode of administration, and use of suitable carrier will depend upon the location, size and type of tumor or infectious lesion.

The present invention provides for both prophylactic and therapeutic methods of treating a subject having a tumor, metastasis, or infectious lesion. Administration of a prophylactic agent can occur prior to the manifestation of symptoms characteristic of the tumor, metastasis, or infectious lesion such that it is prevented or, alternatively, delayed in its progression. The prophylactic agent may also be administered during remission of a tumor or infection, to prevent regrowth of the tumor or further infectious episodes. Administration of a therapeutic agent can occur during tumor growth or infection, to reduce the size, extent, or spread of the tumor or infection.

Toxicity and therapeutic efficacy of DCs and cytokines or nucleic acids encoding cytokines can be determined by standard pharmaceutical procedures in cell cultures or experimental animals, e.g., for determining the Ld₅₀ (the dose lethal to 50% of the population) and the Ed₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. Care should be taken to design a delivery system that targets such DCs and cytokines or nucleic acids encoding a cytokine to the site of affected tissue in order to minimize potential damage to uninfected cells and to reduce side effects.

The data obtained from cell culture assays and animal studies can be used in formulating a range of dosage for use in humans. The dosage of such DCs and cytokines or nucleic acids encoding a cytokine lies preferably within a range of circulating concentrations that include the ED₅₀ with little or no toxicity. The dosage may vary within this range depending upon the dosage form employed and the route of administration utilized. For any DC and cytokine or nucleic acid encoding a cytokine used in the method of the invention, the therapeutically effective dose can be estimated initially from cell culture assays. A dose may be formulated in animal models to achieve a circulating plasma concentration range that includes the IC₅₀ (i.e., the concentration of the DC and cytokine or nucleic acid encoding a cytokine that achieves a half-maximal inhibition of symptoms) as determined in cell culture. Such information can be used to more accurately determine useful doses in humans. Levels in plasma may be measured, for example, by high performance liquid chromatography.

It is contemplated that with regard to mammalian recipients, the DCs and cytokines or nucleic acids encoding a cytokine of interest may be administered by any conventional approach known and/or used in the art. Thus, as appropriate, administration can be oral or parenteral, including intravenous and intraperitoneal routes of administration. In addition, administration can be by periodic injections of a bolus (e.g., directly into the tumor or infectious lesion), or can be made more continuous by intravenous or intraperitoneal administration from a reservoir which is external (e.g., an intravenous bag). Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the cytokine or nucleic acid encoding a cytokine may be in powder form for reconstitution with a suitable vehicle, e.g., sterile pyrogen-free water, before use. In certain embodiments, the compounds of the invention can be therapeutic-grade. That is, certain embodiments comply with standards of purity and quality control required for administration to humans. Veterinary applications are also within the intended meaning as used herein.

The formulations, both for veterinary and for human medical use, of the DCs and cytokines or nucleic acids encoding a cytokine according to the present invention typically include such drugs in association with a pharmaceutically acceptable carrier therefore and optionally other therapeutic ingredient(s). The carrier(s) should be “acceptable” in the sense of being compatible with the other ingredients or cells of the formulations and not deleterious to the recipient thereof. Pharmaceutically acceptable carriers, in this regard, are intended to include any and all solvents, dispersion media, coatings, antibacterial and antifungal agents, isotonic and absorption delaying agents, and the like, compatible with pharmaceutical administration. The use of such media and agents for pharmaceutically active substances is known in the art. Except insofar as any conventional media or agent is incompatible with an active compound, use thereof in the DCs and cytokines or nucleic acids encoding a cytokine compositions is contemplated. Supplementary active compounds also can be incorporated into the compositions. The formulations may conveniently be presented in dosage unit form and may be prepared by any of the methods well known in the art of pharmacy/microbiology. In general, some formulations are prepared by bringing the DCs and cytokines or nucleic acids encoding a cytokine into association with a liquid carrier or a finely divided solid carrier or both, and then, if necessary, shaping the product into the desired formulation.

A pharmaceutical composition of the invention should be formulated to be compatible with its intended route of administration. Examples of routes of administration include oral or parenteral, e.g., intravenous, intraarterial, intradermal, intratumoral, inhalation, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. In a preferred embodiment, the DCs and cytokines or nucleic acids encoding a cytokine are administered in AIM5 medium or other medium compatible with the viability and activity of the DCs.

Useful solutions for oral or parenteral administration can be prepared by any of the methods well known in the pharmaceutical art, described, for example, in Remington's Pharmaceutical Sciences, (Gennaro, A., ed.), Mack Pub., (1990). Formulations for parenteral administration can also include glycocholate for buccal administration, methoxysalicylate for rectal administration, or cutric acid for vaginal administration. The parenteral preparation can be enclosed in ampoules, disposable syringes or multiple dose vials made of glass or plastic. Suppositories for rectal administration also can be prepared by mixing the DCs and cytokines or nucleic acids encoding a cytokine with a non-irritating excipient such as cocoa butter, other glycerides, or other compositions which are solid at room temperature and liquid at body temperatures. Formulations also can include, for example, polyalkylene glycols such as polyethylene glycol, oils of vegetable origin, hydrogenated naphthalenes, and the like. Formulations for direct administration can include glycerol and other compositions of high viscosity. Other potentially useful parenteral carriers for these drugs include ethylene-vinyl acetate copolymer particles, osmotic pumps, implantable infusion systems, and liposomes. Formulations for inhalation administration can contain as excipients, for example, lactose, or can be aqueous solutions containing, for example, polyoxyethylene-9-lauryl ether, glycocholate and deoxycholate, or oily solutions for administration in the form of nasal drops, or as a gel to be applied intranasally. Retention enemas also can be used for rectal delivery.

Formulations of the present invention suitable for oral administration may be in the form of discrete units such as capsules, gelatin capsules, sachets, tablets, troches, or lozenges, each containing a predetermined amount of the cytokine or nucleic acid encoding a cytokine; in the form of a powder or granules; in the form of a solution or a suspension in an aqueous liquid or non-aqueous liquid; or in the form of an oil-in-water emulsion or a water-in-oil emulsion. The cytokines or nucleic acids encoding a cytokine may also be administered in the form of a bolus, electuary or paste. A tablet may be made by compressing or moulding the cytokine or nucleic acids encoding a cytokine optionally with one or more accessory ingredients. Compressed tablets may be prepared by compressing, in a suitable machine, the drug in a free-flowing form such as a powder or granules, optionally mixed by a binder, lubricant, inert diluent, surface active or dispersing agent. Moulded tablets may be made by moulding, in a suitable machine, a mixture of the powdered cytokine or nucleic acid encoding a cytokine and suitable carrier moistened with an inert liquid diluent.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition should be sterile and should be fluid to the extent that easy syringability exists. It should be stable under the conditions of manufacture and storage and should be preserved against the contaminating action of microorganisms such as bacteria and fimgi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Formulations suitable for topical administration, include liquid or semi-liquid preparations such as liniments, lotions, gels, applicants, oil-in-water or water-in-oil emulsions such as creams, ointments or pastes; or solutions or suspensions such as drops. Formulations for topical administration to the skin surface can be prepared by dispersing the cytokine or nucleic acid encoding a cytokine with a dermatologically acceptable carrier such as a lotion, cream, ointment or soap. Particularly useful are carriers capable of forming a film or layer over the skin to localize application and inhibit removal. For topical administration to internal tissue surfaces, the DCs and cytokine or nucleic acid encoding a cytokine can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used.

For inhalation treatments, inhalation of powder (self-propelling or spray formulations) dispensed with a spray can, a nebulizer, or an atomizer can be used. Such formulations can be in the form of a fine powder for pulmonary administration from a powder inhalation device or self-propelling powder-dispensing formulations. In the case of self-propelling solution and spray formulations, the effect may be achieved either by choice of a valve having the desired spray characteristics (i.e., being capable of producing a spray having the desired particle size) or by incorporating the cytokine as a suspended powder in controlled particle size. For administration by inhalation, the compounds also can be delivered in the form of an aerosol spray from pressured container or dispenser that contains a suitable propellant, e.g., a gas such as carbon dioxide, or a nebulizer.

Systemic administration also can be by transmucosal or transdermal means. For transmucosal or transdermal administration, penetrants appropriate to the barrier to be permeated are used in the formulation. Such penetrants generally are known in the art, and include, for example, for transmucosal administration, detergents, bile salts, and filsidic acid derivatives. Transmucosal administration can be accomplished through the use of nasal sprays or suppositories. For transdermal administration, the active DCs and cytokine or nucleic acid encoding a cytokine compounds typically are formulated into ointments, salves, gels, or creams as generally known in the art. A wash solution can be used locally to treat an injury or inflammation to accelerate healing.

The active compounds may be prepared with carriers that will protect the DCs and cytokine or nucleic acid encoding a cytokine against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials also can be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811. Microsomes and microparticles also can be used.

In addition to the formulations described previously, the DCs and cytokine or nucleic acid encoding a cytokine may also be formulated as a depot preparation. Such long acting formulations may be administered by implantation (for example subcutaneously or intramuscularly) or by intramuscular injection. Thus, for example, the cytokine or nucleic acid encoding a cytokine may be formulated with suitable polymeric or hydrophobic materials (for example as an emulsion in an acceptable oil) or ion exchange resins, or as sparingly soluble derivatives, for example, as a sparingly soluble salt. Other suitable delivery systems include microspheres, which offer the possibility of local noninvasive delivery of DCs and cytokine or nucleic acid encoding a cytokine over an extended period of time. This technology utilizes microspheres of precapillary size that can be injected via a coronary catheter into any selected part of the body, e.g., the eye, or other organs without causing inflammation or ischemia. The administered DCs and cytokine or nucleic acid encoding a cytokine therapeutic is slowly released from these microspheres and taken up by surrounding tissue cells.

Where the drug comprises part of a tissue or organ preservation solution, any commercially available preservation solution can be used to advantage. For example, useful solutions known in the art include Collins solution, Wisconsin solution, Belzer solution, Eurocollins solution, and lactated Ringer's solution.

The effective concentration of the DCs and cytokine or nucleic acid encoding a cytokine to be delivered in a therapeutic composition will vary depending upon a number of factors, including the final desired dosage of the DCs and cytokine or nucleic acid encoding a cytokine to be administered and the route of administration. The preferred dosage to be administered also is likely to depend on such variables as the type and extent of tumor or infection to be treated, the overall health status of the particular patient, the relative biological efficacy of the DCs and cytokine or nucleic acid encoding a cytokine delivered, the formulation of the DCs and cytokine or nucleic acid encoding a cytokine, the extent to which regulatory sequences in an expression vector encoding a cytokine cause expression of the cytokine (e.g., high vs. low expression) the presence and types of excipients in the formulation, the level of endogenous expression of the cytokine in vivo and the route of administration. In general terms, the therapy of this invention can be provided to an individual using typical dose units deduced from the earlier-described mammalian studies using non-human primates and rodents.

Active immunostimulatory cytokines used in the methods of the invention also include precursors of the active cytokines. The term precursors refers to a pharmacologically inactive (or partially inactive) derivative of a parent molecule that requires biotransformation, either spontaneous or enzymatic, within the organism to release the active cytokines. Precursors are variations or derivatives of the cytokines of the invention which have groups cleavable under metabolic conditions. Precursors become the active cytokines of the invention that are pharmaceutically active in vivo, when they undergo solvolysis under physiological conditions or undergo enzymatic degradation. Precursor forms often offer advantages of solubility, tissue compatibility, or delayed release in the mammalian organism (see Bundgard, Design of Prodrugs, pp. 7-9, 21-24, Elsevier, Amsterdam (1985); and Silverman, The Organic Chemistry of Drug Design and Drug Action, pp. 352-401, Academic Press, San Diego, Calif. (1992).

In conjunction with such treatment, pharmacogenomics (i.e., the study of the relationship between an individual's genotype and that individual's response to a foreign compound or drug) may be considered. Differences in metabolism of cytokine therapeutics can lead to severe toxicity or therapeutic failure by altering the relationship between dose and blood concentration of the pharmacologically active cytokine(s). Thus, a physician or clinician may consider applying knowledge obtained in relevant pharmacogenomics studies in determining whether to administer a cytokine as well as tailoring the dosage and/or therapeutic regimen of treatment with the cytokine.

With regard to mammals, it is contemplated that the effective dose of a cytokine will be in the range of about 0.01 to about 50 mg/kg, preferably about 0.1 to about 10 mg/kg of body weight, administered in single or multiple doses. Typically, the cytokine may be administered to a human recipient in need of treatment at a daily dose range of about 1 to about 2000 mg per patient.

The DC and immunostimulatory cytokine or nucleic acid encoding an immunostimulatory cytokine therapeutic may be administered alone or in combination with other molecules known to have a beneficial effect on stimulating the immune system, including molecules capable of tissue repair and regeneration and/or inhibiting inflammation. Examples of useful cofactors include basic fibroblast growth factor (bFGF), ciliary neurotrophic factor (CNTF), axokine (a mutein of CNTF), leukemia inhibitory factor (LIF), neutrotrophin 3 (NT-3), neurotrophin-4 (NT-4), nerve growth factor (NGF), insulin-like growth factor II, prostaglandin E2, 30 kD survival factor, taurine, and vitamin A. Other useful cofactors include symptom-alleviating cofactors, including antiseptics, antibiotics, antiviral, antifungal agents, analgesics, and anesthetics.

The DC and immunostimulatory cytokine or nucleic acid encoding an immunostimulatory cytokine therapeutics also may be associated with means for targeting the therapeutic to a desired tissue. Alternatively, an antibody or other binding protein that interacts specifically with a surface molecule on the desired target tissue cells also may be used. Such targeting molecules further may be covalently associated to the cytokine therapeutic, e.g., by chemical crosslinking, or by using standard genetic engineering means to create, for example, an acid labile bond such as an Asp-Pro linkage. Useful targeting molecules may be designed, for example, using the simple chain binding site technology disclosed, for example, in U.S. Pat. No. 5,091,513.

The present invention is further illustrated by the following examples, which should not be construed as limiting in any way. The contents of all references cited throughout this application (including literature references, issued patents, and published patent applications) are hereby expressly incorporated by reference.

### EXEMPLIFICATION

### Example 1

### Materials and Methods

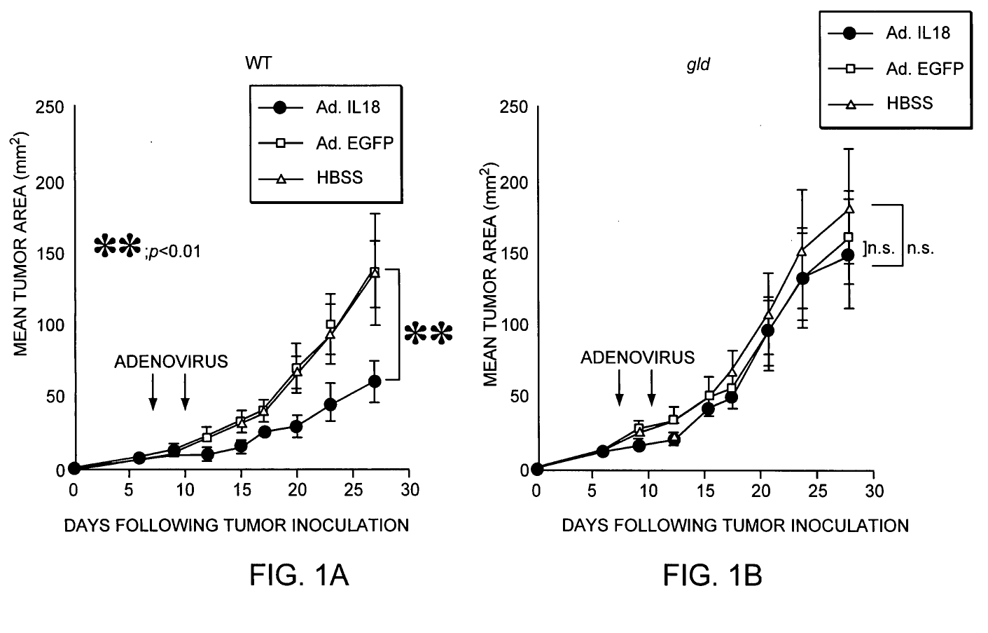

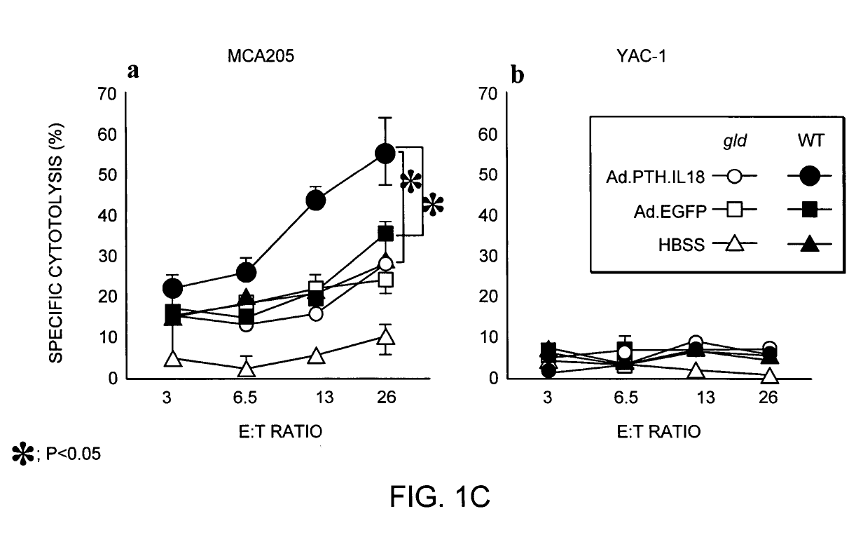

Preparation of Adenoviral Vectors: Construction of the IL-18 vector was described previously (Osaki et al. (1999) supra). Briefly, the fragment encoding the prepro leader peptide of human parathyroid hormone (Ghivizzani et al. (1997) J. Immunol. 159: 3604-12) (Genbank numbers) was fused to the sequence encoding mature IL-18 cDNA, termed PTH.IL-18 (Genbank numbers). The PTH.IL-18 fragment was inserted into a shuttle plasmid (Hardy et al. (1997) J. Virol. 71: 1842-49), termed pAdlox, to make an E1 and E3-substituted recombinant adenoviral vector designated Adlox.PTH.IL-18 (FIG. 1A). A recombinant adenoviral vector encoding PTH.IL-18 was generated through Cre-lox recombination as previously described (Hardy et al. (1997) supra), and was termed Ad.PTH.IL-18. An adenoviral vector expressing enhanced green fluorescent protein (EGFP) (Ad.EGFP) was created in the same manner by subcloning the respective fragments obtained from pEGFP-N1 (Clontech, Palo Alto, Calif.) (Cormack et al. (1996) Gene 177: 33-38) (FIG. 1B). Expression of mIL-18 was determined in an mIL-18 ELISA using MCA205 sarcoma cells (ATCC number) infected at different multiplicities of infection (m.o.i) with Ad.PTH.IL-18 (FIG. 1C), and biological activity was confirmed as described previously (Osaki et al. (1999) supra).

Recombinant cytokines: Murine rIL-18 was obtained from Hayashibara Biochemical Laboratories (Okayama, Japan). Murine rIL-4 and murine GM-CSF were obtained from Schering-Plough Research Institute. rhIL-2 was obtained from Chiron, Emeryville, Calif.

Tumor cell lines, dendritic cells and animal experiments: MCA205, a methylcholanthrene-induced murine fibrosarcoma cell line (ATCC), and MC38, a murine adenocarcinoma cell line (ATCC), were obtained from the National Cancer Institute, Bethesda, Md. YAC-1 cells were obtained from the University of Pittsburgh, Pittsburgh, Pa. (ATCC). These cell lines were maintained in RPMI 1640 supplemented with 10% heat-inactivated fetal bovine serum, 2 mM glutamine, 100 mg/ml streptimycin, 100 IU/ml penicillin, and 5×10⁻⁵ M 2-mercaptoethanol (Life Technologies, Inc., Grand Island, N.Y.), referred to hereinafter as complete medium (CM) (Nishioka (1999) Cancer Res. 59: 4035-41). Bone-marrow derived dendritic cells (BM-DCs) cultured with GM-CSF and IL-4 for 6 days were prepared as previously described (Nishioka (1999) supra). Phenotypic analysis with flow cytometry was performed on all preparations as described below to ensure the quality of the cell preparations. Wild type (WT) C57BL/6 mice were obtained from Taconic Farms (Germantown, N.Y.) and gld (FasL deficient) mice were obtained from the Jackson Laboratory (Bar Harbor, Mass. and used for experiments when they were 7 to 12 weeks old. All the animals were ear-tagged, randomized before experiments, and treated and examined in a blinded fashion, 7 animals per treatment group. 1×10⁵ MCA205 tumor cells were injected i.d. into the flank(s) of mice on day 0. In some experiments, mice received 1×10⁹ plaque forming units (p.f.u.) of adenoviral vector or HBSS as a control on day 7 and day 10. In other experiments, mice received 3×10⁸ p.f.u. of adenoviral vector or HBSS and 1×10⁶ of DC. Tumor size was measured every 3-4 days and expressed as the product of the perpendicular diameters of the individual tumors. On day 14, two animals in each group were sacrificed to harvest samples to be tested in vitro. Each animal experiment was repeated at least twice. Representative results are shown.

Flow cytometry: DCs were stained with phycoerythrin (PE-) or fluorescein isothiocyanate (FITC)-conjugated monoclonal antibodies against murine cell surface molecules CD11c, CD80, CD86, Gr-1, H-2 Kb, I-Ab and appropriate isotype controls (all from PharMingen, San Diego, Calif.). Lymphoid cells harvested from lymph nodes were stained with CD4 and CD8. Cells were examined with the FACScan (Becton Dickinson, Sunnyvale, Calif.).

Cytokine release or CTL assay in vitro: Lymphoid cells were obtained from groin lymph nodes harvested from the mice that had received i.t. injection of adenoviral vector or HBSS, or adenoviral vector or HBSS and DCs 7 days earlier. To examine the IFN-γ response of the lymphoid cells, contaminating erythrocytes were lysed with 0.83M NH4Cl buffer and 2×10⁶ lymphoid cells were co-cultured with 2×10⁵ irradiated (10000 rad) MCA205 cells in the presence of rhIL-2 (25 IU/ml) in 24-well plates for 40 hours. The resultant supernatant was collected and examined with an enzyme linked immunoabsorbent assay (ELISA) specific for mIFN-γ (PharMingen, San Diego, Calif.). To obtain CTLs from these cells, 2×10⁶ lymphoid cells were re-stimulated in vitro with 2×10⁵ irradiated (10000 rad) MCA205 cells in the presence of rhIL-2 (25 IU/ml) for 5 days. Cytotoxic activity was examined using lymphoid cells after co-culture. Viable lymphoid cells were counted using trypan blue-exclusion according to standard methods and used as effector cells for the standard 4 hour−⁵¹Cr release assay against the target cells. In brief, 10⁶ cells of each target were labeled with 100 μCi of Na₂⁵¹CrO₄ for 1 hour, rinsed twice, and plated with target cells at an appropriate E:T ratio in 96-well round bottom plates. The supernatant (100 μl) was collected after a 4 hour incubation and the radioactivity was counted with a y-counter. The percentage of the specific lysis was calculated using the following formula: % specific lysis=100×(experimental release−spontaneous release)/(maximal release−spontaneous release). Forty hours after the culture, 0.4 ml of supernatant was collected for ELISA analysis for IFN-γ (Pharmingen, San Diego, Calif.). The lower detection limit of these assays was 15 pg/ml. For cytolytic assay, cells were cultured for 4 days, collected and tested for cytotoxic activity.

Statistical evaluation: Statistical analyses were performed using the repeated measure ANOVA method when comparing the in vivo tumor growth and cytotoxic activity in an individual group. An unpaired two-tailed Student's t test was used to compare cytokine expression. Differences were considered significant when the p value was less than 0.05.

### Example 2

### Intratumoral Injection of an IL-18 Adenoviral Vector Induces Regression of Established MCA205 Sarcoma in WT Mice, But Not Gld Mice

To examine the anti-tumor effect of i.t., injection of Ad.PTH.IL-18, 1×10⁵ of MCA205 cells were inoculated intradermally (i.d.) in the right flank of mice on day 0. Each group, consisting of 5 mice, was treated with i.t. injection of HBSS, 1×10⁹ p.f.u. Ad.EGFP, or 1×10⁹ p.f.u. Ad.PTH.IL-18 on day 7 and day 10. Significant anti-tumor effects were observed in mice treated with Ad.PTH.IL-18 compared with mice injected with a control adenoviral vector, Ad.EGFP (p=0.0025), or HBSS (p=0.0033) (FIG. 1Ba). In contrast, when gld mice were treated with Ad.PTH.IL-18, no significant anti-tumor effects were observed (p=0.508 against mice treated with Ad.EGFP,p=0.273 against mice treated with HBSS) (FIG. 1Bb). Thus, the Fas-FasL pathway appears to be involved in the anti-tumor effects of Ad.PTH.IL-18.

### Example 3

### Intratumoral Delivery of Ad.PTH.IL-18 Enhances the Tumor Specific CTL Response in Regional Lymph Nodes of WT but Not of Gld Mice

The in vitro cytotoxic activity of lymphoid cells harvested from the regional lymph nodes of treated animals and the mechanism responsible for the anti-tumor effect of i.t. injection of Ad.PTH.IL-18 were examined. Animals were sacrificed 7 days after i.t. injection, and draining lymph nodes were harvested and processed to obtain lymphoid cells. These lymphoid cells were cultured in vitro with irradiated MCA205 cells to examine the IFN—Y response and in vitro cytolytic activity against MCA205 cells and YAC-1 cells. In immunocompetent mice, i.t. injection of Ad.PTH.IL-18 enhanced tumor specific IFN-γ responses (1102±129 pg/ml) and the cytolytic activity against MCA205 but not against YAC-1 cells (FIG. 1C). In gld mice, i.t. injection of Ad.PTH.IL-18 enhanced neither tumor specific IFN-γ responses (137±11 μg/ml) nor in vitro cytotoxic activity (FIG. 1C). These data suggest that the i.t. injection of Ad.PTH.IL-18 significantly enhanced the cellular immune response to MCA205 cells in WT mice, but not in gld mice.

### Example 4

### Intratumoral Injection of Both Ad.PTH.IL-18 and DC a Induces Strong Anti-Tumor Effect In Vivo

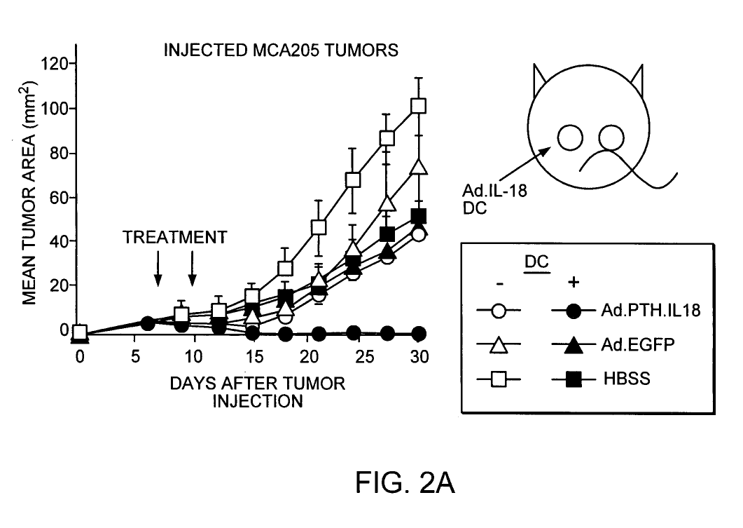

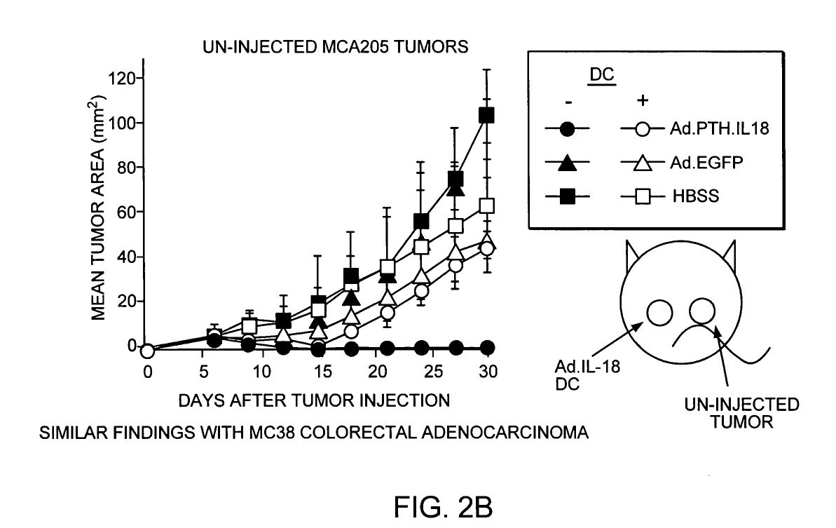

Tumor cells killed by IL-18-activated NK cells can serve as an antigen source for DCs to rapidly induce tumor specific CTL (Tanaka et al. (2000) Cancer Res. 60: 4838-44). Thus, the in vivo anti-tumor effect of i.t. injection of both Ad.PTH.IL-18 and DC against MCA205 fibrosarcoma cells was examined. Although the dose of 3×10⁸ p.f.u. of Ad.PTH.IL-18 alone could not achieve a significant anti-tumor effect on MCA205 cells, the tumors treated with Ad.PTH.IL-18 and DC were completely abrogated after day 18 (FIG. 2A). DC alone, Ad.PTH.IL-18 alone or DC plus control vector showed significant but modest anti-tumor effects.

In addition, un-injected tumor located at a distant site in the same animal was completely rejected only with the treatment consisting of DC plus Ad.PTH.IL-18 (FIG. 2B). Similar anti-tumor responses were observed in MC38 cells, another syngeneic mouse tumor system. Coinjection of Ad.PTH.IL-18 and DC therefore induces potent anti-tumor effects against not only the injected tumor but also against distant tumors such as metastases.

### Example 5

### The Cellular Immune Response Associated With the Treatment With Ad.PTH.IL-18 and DC is Specific for the Treated Tumor, MCA205, and is MHC Class-I Restricted

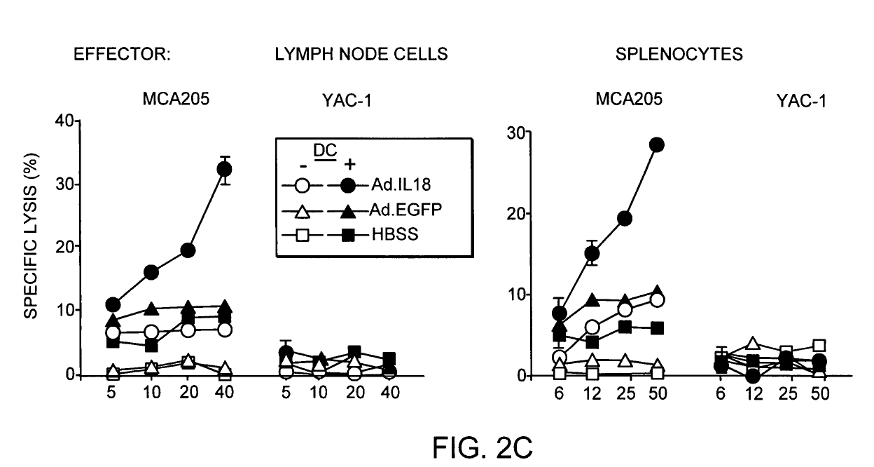

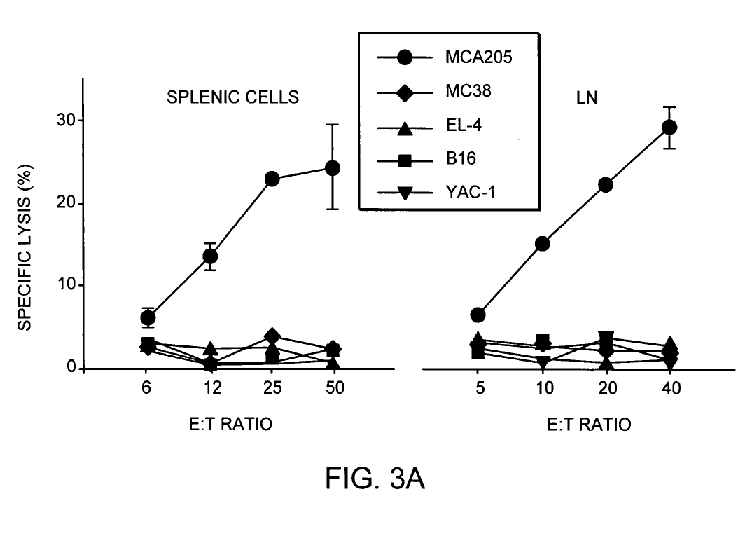

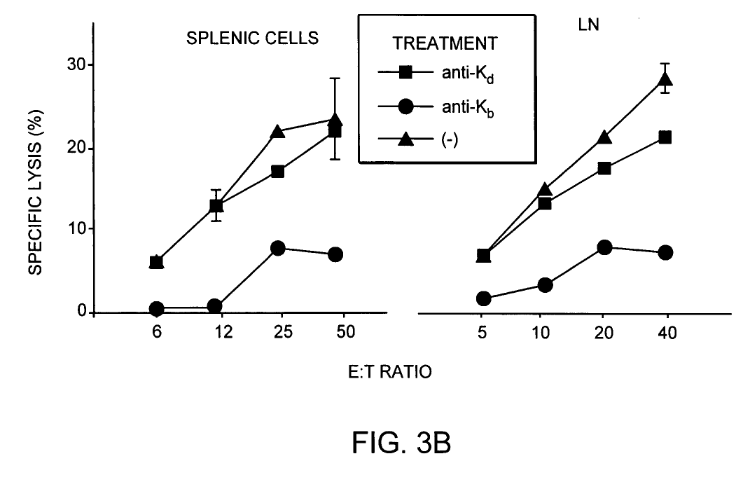

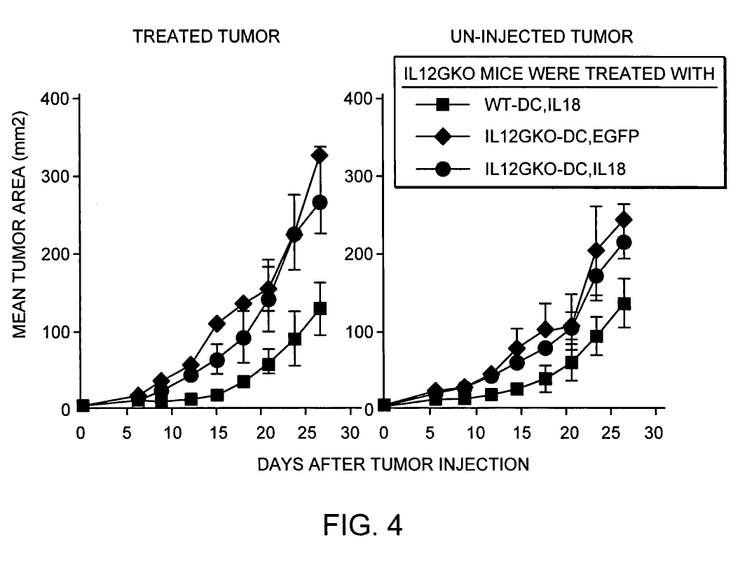

To investigate the nature of anti-tumor immune response, lymphoid cells were collected from lymph nodes and spleens of the Ad.PTH.IL-18 and DC treated animals and the specificity and MHC class-I restriction of effector cells against tumor cells was examined. The most potent cytolytic activity of those effector cells was generated against MCA205 cells when the animals were treated with Ad.PTH.IL-18 and DC. Treatment with DC alone, DC plus control vector, or Ad.IL-18 alone showed significant but modest increases of cytolysis (FIG. 2C). To analyze the specificity of effector cells generated from the lymphoid cells of the animals treated with Ad.PTH.IL-18 and DC, the cytolytic activity of effector cells was assayed against four different syngeneic tumor cell lines (MCA205, MC38, EL-4, B116), and YAC-1 cells (a NK sensitive cell line). The cytotoxicity of the effector cells was significantly enhanced only against MCA205 cells but not significantly against any other cell lines (p<0.01 for all) (FIG. 3A). Thus, the anti-tumor effects against the tumor at a distant site appeared to be mediated by the tumor-specific effector cells generated by the injection of both Ad.PTH.IL-18 and DC. When the effector cells were incubated with anti-MHC class I antibody, the cytolytic activity was significantly inhibited (FIG. 3B). These results strongly suggest that the cytolytic activity of the effector cells was MCA205-specific and MHC class I-restricted.

### Example 6

### Coinjection of Ad.PTH.IL-18 and DC Can be an Effective Treatment for Treatment Against Another Tumor Cell Line, the MC38 Adenocarcinoma Cell

The efficiency of i.t. injection of both Ad.PTH.IL-18 and DC on MC38 adenocarcinoma cells was analyzed. As shown in FIG. 3A, significant anti-tumor effects were observed on the injected tumor with i.t. coinjection of Ad.PTH.IL-18 and DC, and the effects were superior than those in mice treated with DC alone (p<0.001), DC plus control vector (p<0.001), or Ad.PTH.IL-18 alone (p<0.01). The anti-tumor effect was also observed against a non-injected distant tumor of the treated animals (FIG. 3B). These results suggest that i.t. coinjection using Ad.PTH.IL-18 and DC can be an effective therapy even against weakly immunogenic tumors such as MC38 adenocarcinoma.

These results suggest that the NK activity through the Fas-FasL system, which requires direct effector-target contact, plays an important role for the generation of potent CTLs specific to the tumor both in vitro and in vivo.

In the studies of coinjection of Ad.PTH.IL-18 and DC, ⅓ the amount of adenoviral vector achieved sufficient anti-tumor effect compared to adenoviral vector alone. Although a significantly lower dose (⅓) of adenoviral vector was injected with DCs, coinjection of Ad.PTH.IL-18 and DC was associated with more effective anti-tumor response when compared with that of Ad.PTH.IL-18 injection alone. There, DCs appear to play an important role as primary antigen-presenting cells to initiate and maintain T-cell responses acquiring tumor cells damaged with locally expressed IL-18 through enhanced NK cell activity (Hashimoto et al. (1999) J. Immunol. 163: 583-9; Osaki et al. (1998) J. Immunol. 160: 1742-49; Tanaka et al. (2000) supra). Not to be limited to any particular theory, inoculated immature DCs may acquire damaged tumor cells, become matured and express CCR on the surface (Hirao et al. (2000) Cancer Res. 60: 2209-17), and migrate into drainage lymph nodes. Then, DCs in the T cell area of the regional lymph nodes may activate and educate naïve T cells into tumor specific CTLs (Steinman (1991) Ann. Rev. Immunol. 9: 271-96; Mayordomo et al. (1995) Nature Med. 1: 1297-302; Zitvogel et al. (1996) J. Exp. Med. 183: 87-97; Porgador et al. (1996) J. Immunol. 156: 2918-26). This hypothesis is also consistent with previous reports of co-culture system using NK cells, DCs, T cells, live tumor cells, and IL-18 protein in vitro (Tanaka et al. (2000) supra).

To examine the involvement of endogenous IL-12 in the potent anti-tumor effects mediated by Ad.PTH.IL-18 and DC, DCs cultured from IL-12 gene deficient (IL-12 GKO) mice were used. Coinjection of Ad.PTH.IL-18 and DC from IL-12 GKO mice was associated with significantly less anti-tumor effects when compared with that of the treatment with DCs from immunocompetent animals (FIG. 5). This suggested that the anti-tumor effect induced with coinjection of Ad.PTH.IL-18 and DC plays important role for inducing adoptive immune response with the help of endogenous IL-12. This observation is consistent with the reported synergistic effect between IL-18 and IL-12 in the multiple systems including fungus infection (Zhang et al. (1997) Infect. Immun. 65: 3594-99), Toxoplasma gondii infection (Cai et al. (2000) Infect. Immun. 68: 6932-38), angiogenesis inhibition in a murine tumor model (Coughlin et al. (1998) J. Clin. Invest. 101: 1441-52) and effective activation of CD8 positive T cells (Okamoto et al. (1999) J. Immunol. 162: 3202-11).

### Equivalents

The invention may be embodied in other specific forms without departing from the spirit or essential characteristics thereof. The foregoing embodiments are therefore to be considered in all respects illustrative rather than limiting of the invention described herein.

What is claimed is:

1. A method of inhibiting or treating a tumor or infectious lesion in a subject, comprising:
administering into or near a site of a tumor or infectious lesion in a subject an effective amount of an antigen presenting cell and an immunostimulatory cytokine or a nucleic acid encoding an immunostimulatory cytokine.

2. The method of claim 1, wherein the antigen presenting cell is a dendritic cell.

3. The method of claim 2, wherein the dendritic cell is selected from the group consisting of a CD34+-derived dendritic cell, a bone marrow-derived dendritic cell, a monocyte-derived dendritic cell, a splenocyte derived dendritic cell, a skin-derived dendritic cell, a follicular dendritic cell, and a germinal center dendritic cell.

4. The method of claim 1, wherein the dendritic cell is a CD34+-derived dendritic cell cultured in the presence of at least one factor selected from the group consisting of granulocyte colony stimulating factor, granulocyte macrophage colony stimulatory factor, tumor necrosis factor alpha, interleukin 4, the Flt-3 ligand, and the kit ligand.

5. The method of claim 1, wherein the antigen presenting cell is selected from a group consisting of a Langherhans' cell, an interdigitating cell, a B cell, and a macrophage.

6. The method of claim 1, wherein the immunostimulatory cytokine is selected from the group consisting of interleukin-1 a, interleukin-1 P, interleukin-2, interleukin-3, interleukin-4, interleukin-6, interleukin-8, interleukin-9, interleukin-10, interleukin-12, interleukin-18, interleukin-19, interleukin-20, interleukin-23, interleukin-27, interleukin-1f3, interleukin-1f5, interleukin-1f6, interleukin-1f7, interleukin-1f8, interleukin-1f9, interleukin-1f10, interferon-α, interferon-β, interferon-γ, tumor necrosis factor α, transforming growth factor, granulocyte colony stimulating factor, macrophage colony stimulating factor, granulocyte-macrophage colony stimulating factor, the Flt-3 ligand, and the kit ligand.

7. The method of claim 1, wherein the expression vector is a viral vector.

8. The method of claim 2, wherein the expression vector is selected from the group consisting of an adenoviral vector, an adeno-associated viral vector, a retroviral vector, a lentiviral vector, a herpes viral vector, and a vaccinia viral vector.

9. The method of claim 1, wherein the subject has a tumor selected from the group consisting of melanoma, hepatoma, adenocarcinoma, colorectal cancer, basal cell cancer, oral cancer, nasopharyngeal cancer, laryngeal cancer, bladder cancer, head and neck cancer, renal cell cancer, pancreatic cancer, pulmonary cancer, cervical cancer, ovarian cancer, esophageal cancer, gastric cancer, prostate cancer, testicular cancer, and breast cancer.

10. The method of claim 1, wherein the size of the tumor or infectious lesion is decreased.

11. The method of claim 1, wherein said administering step comprises injecting into the tumor or infectious lesion.

12. The method of claim 1, wherein said administering step comprises injecting the subject within the same organ as the tumor or infectious lesion.

13. A method of inhibiting or treating metastasis of a tumor in a subject, comprising:
administer into or near a site of a tumor in a subject an effective amount of an antigen presenting cell and an immunostimulatory cytokine or a nucleic acid encoding an immunostimulatory cytokine.

14. The method of claim 13, wherein the antigen presenting cell is a dendritic cell.

15. The method of claim 14, wherein the dendritic cell is selected from the group consisting of a CD34+-derived dendritic cell, a bone marrow-derived dendritic cell, a monocyte-derived dendritic cell, a splenocyte derived dendritic cell, a skin-derived dendritic cell, a follicular dendritic cell, and a germinal center dendritic cell.

16. The method of claim 13, wherein the dendritic cell is a CD34+-derived dendritic cell cultured in the presence of at least one factor selected from the group consisting of granulocyte colony stimulating factor, granulocyte macrophage colony stimulatory factor, tumor necrosis factor alpha, interleukin 4, the Flt-3 ligand, and the kit ligand.

17. The method of claim 13, wherein the antigen presenting cell is selected from a group consisting of a Langherhans' cell, an interdigitating cell, a B cell, and a macrophage.

18. The method of claim 13, wherein the immunostimulatory cytokine is selected from the group consisting of interleukin-1α, interleukin-1β, interleukin-2, interleukin-3, interleukin-4, interleukin-6, interleukin-8, interleukin-9, interleukin-10, interleukin-12, interleukin-18, interleukin-19, interleukin-20, interleukin-23, interleukin-27, interleukin-1f3, interleukin-1f5, interleukin-1f6, interleukin-1f7, interleukin-1f8, interleukin-1f9, interleukin-1f10, interferon-α, interferon-β, interferon-γ, tumor necrosis factor α, transforming growth factor-β, granulocyte colony stimulating factor, macrophage colony stimulating factor, granulocyte-macrophage colony stimulating factor, the Flt-3 ligand, and the kit ligand.

19. The method of claim 13, wherein the expression vector is a viral vector.

20. The method of claim 13, wherein the expression vector is selected from the group consisting of an adenoviral vector, an adeno-associated viral vector, a retroviral vector, a lentiviral vector, a herpes viral vector, and a vaccinia viral vector.

21. The method of claim 13, wherein the subject has a tumor selected from the group consisting of melanoma, hepatoma, adenocarcinoma, colorectal cancer, basal cell cancer, oral cancer, nasopharyngeal cancer, laryngeal cancer, bladder cancer, head and neck cancer, renal cell cancer, pancreatic cancer, pulmonary cancer, cervical cancer, ovarian cancer, esophageal cancer, gastric cancer, prostate cancer, testicular cancer, and breast cancer.

22. The method of claim 13, wherein the size of the tumor or infectious lesion is decreased.

23. The method of claim 13, wherein the size of the metastasis is decreased.

24. The method of claim 13, wherein the number of the metastases is decreased.

25. The method of claim 13, wherein said administering step comprises injecting into the tumor or infectious lesion.

26. The method of claim 13, wherein said administering step comprises injecting the subject within the same organ as the tumor or infectious lesion.

27. A therapeutic composition comprising an antigen presenting cell and an immunostimulatory cytokine or a nucleic acid encoding an immunostimulatory cytokine.


SEQUENCE LISTING (87 sequences)

SEQ ID NO: 1 - DNA, 1314 bp, Mus musculus
ggggggggggatttagagacttgctcttgcactaccaaagccacaaagcagccttgcagaaaagagagctccatcatgcctggctcagcactgctatgctgcctgctcttactgactggcatgaggatcagcaggggccagtacagccgggaagacaataactgcacccacttcccagtcggccagagccacatgctcctagagctgcggactgccttcagccaggtgaagactttctttcaaacaaaggaccagctggacaacatactgctaaccgactccttaatgcaggactttaagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtcatcgatttctcccctgtgaaaataagagcaaggcagtggagcaggtgaagagtgattttaataagctccaagaccaaggtgtctacaaggccatgaatgaatttgacatcttcatcaactgcatagaagcatacatgatgatcaaaatgaaaagctaaaacacctgcagtgtgtattgagtctgctggactccaggacctagacagagctctctaaatctgatccagggatcttagctaacggaaacaactccttggaaaacctcgtttgtacctctctccgaaatatttattacctctgatacctcagttcccattctatttattcactgagcttctctgtgaactatttagaaagaagcccaatattataattttacagtatttattatttttaacctgtgtttaagctgtttccattggggacactttatagtatttaaagggagattatattatatgatgggaggggttcttccttgggaagcaattgaagcttctattctaaggctggccacacttgagagctgcagggccctttgctatggtgtcctttcaattgctctcatccctgagttcagagctcctaagagagttgtgaagaaactcatgggtcttgggaagagaaaccagggagatcctttgatgatcattcctgcagcagctcagagggttcccctactgtcatcccccagccgcttcatccctgaaaactgtggccagtttgttatttataaccacctaaaattagttctaatagaactcatttttaactagaagtaatgcaattcctctgggaatggtgtattgtttgtctgcctttgtagcagcatctaattttgaataaatggatcttattcg

SEQ ID NO: 2 - protein, 178 aa, Mus musculus
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 3 - DNA, 674 bp, Homo sapiens
gatccaaacatgagccgcctgcccgtcctgctcctgctccaactcctggtccgccccggactccaagctcccatgacccagacaacgtccttgaagacaagctgggttaactgctctaacatgatcgatgaaattataacacacttaaagcagccacctttgcctttgctggacttcaacaacctcaatggggaagaccaagacattctgatggaaaataaccttcgaaggccaaacctggaggcattcaacagggctgtcaagagtttacagaacgcatcagcaattgagagcattcttaaaaatctcctgccatgtctgcccctggccacggccgcacccacgcgacatccaatccatatcaaggacggtgactggaatgaattccggaggaaactgacgttctatctgaaaacccttgagaatgcgcaggctcaacagacgactttgagcctcgcgatcttttagtccaacgtccagctcgttctctgggccttctcaccacagcgcctcgggacatcaaaaacagcagaacttctgaaacctctgggtcatctctcacacattccaggaccagaagcatttcaccttttcctgcggcatcagatgaattgttaattatctaatttctgaaatgtgcagctcccatttggccttgtgcggttgtgttctca

SEQ ID NO: 4 - protein, 152 aa, Homo sapiens
MSRLPVLLLLQLLVRPGLQAPMTQTTSLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 5 - DNA, 614 bp, Homo sapiens
gatcgttagcttctcctgataaactaattgcctcacattgtcactgcaaatcgacacctattaatgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctgaatattttaatttatgagtttttgatagctttattttttaagtatttatatatttataactcatcataaaataaagtatatatagaatct

SEQ ID NO: 6 - protein, 153 aa, Homo sapiens [sequence omitted]

SEQ ID NO: 7 - DNA, 1604 bp, Homo sapiens
gaattcctctggtcctcatccaggtgcgcgggaagcaggtgcccaggagagaggggataatgaagattccatgctgatgatcccaaagattgaacctgcagaccaagcgcaaagtagaaactgaaagtacactgctggcggatcctacggaagttatggaaaaggcaaagcgcagagccacgccgtagtgtgtgccgccccccttgggatggatgaaactgcagtcgcggcgtgggtaagaggaaccagctgcagagatcaccctgcccaacacagactcggcaactccgcggaagaccagggtcctgggagtgactatgggcggtgagagcttgctcctgctccagttgcggtcatcatgactacgcccgcctcccgcagaccatgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactgaaaaatatggagtggcaatatagaaacacgaactttagctgcatcctccaagaatctatctgcttatgcagtttttcagagtggaatgcttcctagaagttactgaatgcaccatggtcaaaacggattagggcatttgagaaatgcatattgtattactagaagatgaatacaaacaatggaaactgaatgctccagtcaacaaactatttcttatatatgtgaacatttatcaatcagtataattctgtactgatttttgtaagacaatccatgtaaggtatcagttgcaataatacttctcaaacctgtttaaatatttcaagacattaaatctatgaagtatataatggtttcaaagattcaaaattgacattgctttactgtcaaaataattttatggctcactatgaatctattatactgtattaagagtgaaaattgtcttcttctgtgctggagatgttttagagttaacaatgatatatggataatgccggtgagaataagagagtcataaaccttaagtaagcaacagcataacaaggtccaagatacctaaaagagatttcaagagatttaattaatcatgaatgtgtaacacagtgccttcaataaatggtatagcaaatgttttgacatgaaaaaaggacaatttcaaaaaaataaaatgcaaatttttttaaa

SEQ ID NO: 8 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 9 - DNA, 515 bp, Artificial sequence
  Viral IL-10
ccatggagcgaaggttagtggtcactctgcagtgcctggtgctgctttacctggcacctgagtgtggaggtacagaccaatgtgacaattttccccaaatgttgagggacctaagagatgccttcagtcgtgttaaaacctttttccagacaaaggacgaggtagataaccttttgctcaaggagtctctgctagaggactttaagggctaccttggatgccaggccctgtcagaaatgatccaattctacctggaggaagtcatgccacaggctgaaaaccaggacccggaggctaaggaccatgtcaattctttgggtgaaaatctaaagaccctacggctccgcctgcgcaggtgccacaggttcctgccgtgtgagaacaagagtaaagctgtggaacagataaaaaatgcctttaacaagctgcaggaaaaaggaatttacaaagccatgagtgaatttgacatttttattaactacatagaagcatacatgacaattaaagccaggtga

SEQ ID NO: 10 - protein, 172 aa, Artificial Sequence
  Viral IL-10
MERRLVVTLQCLVLLYLAPECGGDNQCDNFPQMLRDLRDAFSRVLTFFQYLCEVDNLLLKESLLGCFLGYLGCQALSEMIQFQLEEVMPQAENQNPEALNHVNSLGEALLQLRLRLRRCHRFLPCENLSLAVEQILDAFDKLQELGIQLAMSEFCIFINQIEAQMYMYILAR

SEQ ID NO: 11 - DNA, 2527 bp, Homo Sapiens
acctccctccgcggagcagccagacagcgagggccccggccgggggcaggggggacgccccgtccggggcaccccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgccccgcgaggaggcaggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggagcctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgccgctgctgctaccgctgctgtggctactggtgctgacgcctggcccgccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctctacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggaggagctgtccaacatgatcgtgcgctcctgcaagtgcagctgaggtcccgccccgccccgccccgcccggccccaccccgccccgcccccgctgccttgcccatgggggctgtatttaaggacaccgtgccccaagcccacctggggccccattaaagatggagagaggactgcggatctctgtgtcattgggcgcctgcctggggtctccatccctgacgttcccccactcccactccctctctctccctctctgcctcctcctgcctgtctgcactattcctttgcccggcatcaaggcacaggggaccagtggggaacactactgtagttagatctatttattgagcaccttgggcactgttgaagtgccttacattaatgaactcattcagtcaccatagcaacactctgagatggcagggactctgataacacccattttaaaggttgaggaaacaagcccagagaggttaagggaggagttcctgcccaccaggaacctgctttagtgggggatagtgaagaagacaataaaagatagtagttcaggcca

SEQ ID NO: 12 - protein, 390 aa, Homo Sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 13 - DNA, 1695 bp, Homo Sapiens
caagcaggatacgtttttctgttgggcattgactagattgtttgcaaaagtttcgcatcaaaaacaaacaacaacaacaaaaaaccaaacaactctccttgatctatactttgagaattgttgatttctttttttttattctgacttttaaaaacaacttttttttccacttttttaaaaaatgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaaatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaaaattcttggaaaagtggcaagaccaaaatgacaatgatgatgataatgatgatgacgacgacaacgatgatgcttgtaacaagaaaacataagagagccttggttcatcagtgttaaaaaatttttgaaaaggcggtactagttcagacactttggaagtttgtgttctgtttgttaaaactggcatctgacacaaaaaaagttgaaggccttattctacatttcacctactttgtaagtgagagagacaagaagcaaatttttttaaa

SEQ ID NO: 14 - protein, 414 aa, Homo Sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 15 - DNA, 2574 bp, Homo Sapiens
  CDS (254)..(1492)
cctgtttagacacatggacaacaatcccagcgctacaaggcacacagtccgcttcttcgtcctcagggttgccagcgcttcctggaagtcctgaagctctcgcagtgcagtgagttcatgcaccttcttgccaagcctcagtctttgggatctggggaggccgcctggttttcctccctccttctgcacgtctgctggggtctcttcctctccaggccttgccgtccccctggcctctcttcccagctcacacatgaagatgcacttgcaaagggctctggtggtcctggccctgctgaactttgccacggtcagcctctctctgtccacttgcaccaccttggacttcggccacatcaagaagaagagggtggaagccattaggggacagatcttgagcaagctcaggctcaccagcccccctgagccaacggtgatgacccacgtcccctatcaggtcctggccctttacaacagcacccgggagctgctggaggagatgcatggggagagggaggaaggctgcacccaggaaaacaccgagtcggaatactatgccaaagaaatccataaattcgacatgatccaggggctggcggagcacaacgaactggctgtctgccctaaaggaattacctccaaggttttccgcttcaatgtgtcctcagtggagaaaaatagaaccaacctattccgagcagaattccgggtcttgcgggtgcccaaccccagctctaagcggaatgagcagaggatcgagctcttccagatccttcggccagatgagcacattgccaaacagcgctatatcggtggcaagaatctgcccacacggggcactgccgagtggctgtcctttgatgtcactgacactgtgcgtgagtggctgttgagaagagagtccaacttaggtctagaaatcagcattcactgtccatgtcacacctttcagcccaatggagatatcctggaaaacattcacgaggtgatggaaatcaaattcaaaggcgtggacaatgaggatgaccatggccgtggagatctggggcgcctcaagaagcagaaggatcaccacaaccctcatctaatcctcatgatgattcccccacaccggctcgacaacccgggccaggggggtcagaggaagaagcgggctttggacaccaattactgcttccgcaacttggaggagaactgctgtgtgcgccccctctacattgacttccgacaggatctgggctggaagtgggtccatgaacctaagggctactatgccaacttctgctcaggcccttgcccatacctccgcagtgcagacacaacccacagcacggtgctgggactgtacaacactctgaaccctgaagcatctgcctcgccttgctgcgtgccccaggacctggagcccctgaccatcctgtactatgttgggaggacccccaaagtggagcagctctccaacatggtggtgaagtcttgtaaatgtagctgagaccccacgtgcgacagagagaggggagagagaaccaccactgcctgactgcccgctcctcgggaaacacacaagcaacaaacctcactgagaggcctggagcccacaaccttcggctccgggcaaatggctgagatggaggtttccttttggaacatttctttcttgctggctctgagaatcacggtggtaaagaaagtgtgggtttggttagaggaaggctgaactcttcagaacacacagactttctgtgacgcagacagaggggatggggatagaggaaagggatggtaagttgagatgttgtgtggcaatgggatttgggctaccctaaagggagaaggaagggcagagaatggctgggtcagggccagactggaagacacttcagatctgaggttggatttgctcattgctgtaccacatctgctctagggaatctggattatgttatacaaggcaagcatttttttttttaaagacaggttacgaagacaaagtcccagaattgtatctcatactgtctgggattaagggcaaatctattacttttgcaaactgtcctctacatcaattaacatcgtgggtcactacagggagaaaatccaggtcatgcagttcctggcccatcaactgtattgggccttttggatatgctgaacgcagaagaaagggtggaaatcaaccctctcctgtctgccctctgggtccctcctctcacctctccctcgatcatatttccccttggacacttggttagacgccttccaggtcaggatgcacatttctggattgtggttccatgcagccttggggcattatgggtcttcccccacttcccctccaagaccctgtgttcatttggtgttcctggaagcaggtgctacaacatgtgaggcattcggggaagctgcacatgtgccacacagtgacttggccccagacgcatagactgaggtataaagacaagtatgaatattactctcaaaatctttgtataaataaatatttttggggcatcctggatgatttcatcttctggaatattgtttctagaacagtaaaagccttattctaaggtg

SEQ ID NO: 16 - protein, 412 aa, Homo Sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 17 - DNA, 2064 bp, Homo sapiens
gaattccgcaggcgctcggggttggagccagcgaccgtcggtagcagcatggctctcctctttctcctaccccttgtcatgcagggtgtgagcagggctgagatgggcaccgcggatctggggccgtcctcagtgcctacaccaactaatgttacaattgaatcctataacatgaaccctatcgtatattgggagtaccagatcatgccacaggtccctgtttttaccgtagaggtaaagaactatggtgttaagaattcagaatggattgatgcctgcatcaatatttctcatcattattgtaatatttctgatcatgttggtgatccatcaaattctctttgggtcagagttaaagccagggttggacaaaaagaatctgcctatgcaaagtcagaagaatttgctgtatgccgagatggaaaaattggaccacctaaactggatatcagaaaggaggagaagcaaatcatgattgacatatttcacccttcagtttttgtaaatggagacgagcaggaagtcgattatgatcccgaaactacctgttacattagggtgtacaatgtgtatgtgagaatgaacggaagtgagatccagtataaaatactcacgcagaaggaagatgattgtgacgagattcagtgccagttagcgattccagtatcctcactgaattctcagtactgtgtttcagcagaaggagtcttacatgtgtggggtgttacaactgaaaagtcaaaagaagtttgtattaccattttcaatagcagtataaaaggttctctttggattccagttgttgctgctttactactctttctagtgcttagcctggtattcatctgtttttatattaagaaaattaatccattgaaggaaaaaagcataatattacccaagtccttgatctctgtggtaagaagtgctactttagagacaaaacctgaatcaaaatatgtatcactcatcacgtcataccagccattttccttagaaaaggaggtggtctgtgaagagccgttgtctccagcaacagttccaggcatgcataccgaagacaatccaggaaaagtggaacatacagaagaactttctagtataacagaagtggtgactactgaagaaaatattcctgacgtggtcccgggcagccatctgactccaatagagagagagagttcttcacctttaagtagtaaccagtctgaacctggcagcatcgctttaaactcgtatcactccagaaattgttctgagagtgatcactccagaaatggttttgatactgattccagctgtctggaatcacatagctccttatctgactcagaatttcccccaaataataaaggtgaaataaaaacagaaggacaagagctcataaccgtaataaaagcccccacctcctttggttatgataaaccacatgtgctagtggatctacttgtggatgatagcggtaaagagtccttgattggttatagaccaacagaagattccaaagaattttcatgagatcagctaagttgcaccaactttgaagtctgattttcctggacagttttctgctttaatttcatgaaaagattatgatctcagaaattgtatcttagttggtatcaaccaaatggagtgacttagtgtacatgaaagcgtaaagaggatgtgtggcattttcacttttggcttgtaaagtacagacttttttttttttttaaacaaaaaaagcattgtaacttatgaacctttacatccagataggttaccagtaacggaacatatccagtactcctggttcctaggtgagcaggtgatgccccagggacctttgtagccacttcactttttttcttttctctgccttggtatagcatatgtgttttgtaagtttatgcatacagtaattttaagtaatttcagaagaaattctcgaagcttttcaaaattggacttaaaatctaattcaaactaatagaattaatggaatatgtaaatagaaacgtgtatattttttatgaaacattacagttagagatttttaaataaagaattttaaaactc

SEQ ID NO: 18 - protein, 489 aa, Homo sapiens
MALLFLLPLVMQGVSRAEMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 19 - DNA, 861 bp, Homo Sapiens
  CDS (49)..(861)
gaattccgcaggcgctcggggttggagccagcgaccgtcggtagcagcatggctctcctctttctcctaccccttgtcatgcagggtgtgagcagggctgagatgggcaccgcggatctggggccgtcctcagtgcctacaccaactaatgttacaattgaatcctataacatgaaccctatcgtatattgggagtaccagatcatgccacaggtccctgtttttaccgtagaggtaaagaactatggtgttaagaattcagaatggattgatgcctgcatcaatatttctcatcattattgtaatatttctgatcatgttggtgatccatcaaattctctttgggtcagagttaaagccagggttggacaaaaagaatctgcctatgcaaagtcagaagaatttgctgtatgccgagatggaaaaattggaccacctaaactggatatcagaaaggaggagaagcaaatcatgattgacatatttcacccttcagtttttgtaaatggagacgagcaggaagtcgattatgatcccgaaactacctgttacattagggtgtacaatgtgtatgtgagaatgaacggaagtgagatccagtataaaatactcacgcagaaggaagatgattgtgacgagattcagtgccagttagcgattccagtatcctcactgaattctcagtactgtgtttcagcagaaggagtcttacatgtgtggggtgttacaactgaaaagtcaaaagaagtttgtattaccattttcaatagcagtataaaaggttctctttggattccagttgttgctgctttactactctttctagtgcttagcctggtattcatctgtttttatatttga

SEQ ID NO: 20 - protein, 270 aa, Homo Sapiens
MALLFLLPLVMQGVSRAEMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGSLWIPVVAALLLFLVLSLVFICFYI

SEQ ID NO: 21 - DNA, 1890 bp, Mus musculus
  misc_feature (1390)..(1390): n = a, t, c or g
gatcctgttgggttttactctactccctgaggacctcagcacatttgccccccagccatggcttgtcttggactccggaggtacaaagctcaactgcagctgccttctaggacttggccttttgtagccctgctcactcttcttttcatcccagtcttctctgaagccatacaggtgacccaaccttcagtggtgttggctagcagccatggtgtcgccagctttccatgtgaatattcaccatcacacaacactgatgaggtccgggtgactgtgctgcggcagacaaatgaccaaatgactgaggtctgtgccacgacattcacagagaagaatacagtgggcttcctagattaccccttctgcagtggtacctttaatgaaagcagagtgaacctcaccatccaaggactgagagctgttgacacgggactgtacctctgcaaggtggaactcatgtacccaccgccatactttgtgggcatgggcaacgggacgcagatttatgtcattgatccagaaccatgcccggattctgacttcctcctttggatccttgtcgcagttagcttggggttgtttttttacagtttcctggtctctgctgtttctttgagcaagatgctaaagaaaagaagtcctcttacaacaggggtctatgtgaaaatgcccccaacagagccagaatgtgaaaagcaatttcagccttattttattcccatcaactgaaaggccgtttatgaagaagaaggagcatacttcagtctctaaaagctgaggcaatttcaactttccttttctctccagctatttttacctgtttgtatattttaaggagagtatgcctctctttaatagaaagctggagcaaaattccaattaagcatactacaatttaaagctaaggagcagaacagagagctgggatatttctgttgtgtcagaaccattttactaaaagcatcacttggaagcagcataaggatatagcattatggtgtggggtcaagggaacattagggaatggcacagcccaaagaaaggaagggggtgaaggaagagattatattgtacacatcttgtatttacctgagagggggtgaaggaagagattatattgtacacatcttgtatttacctgagagatgtttatgacttaaataatttttaaatttttcatgctgttattttctttaacaatgtataattacacgaaggtttaaacatttattcacagagactatgtgacatagccagtggttccaaaggttgtagtgttccaagatgtatttttaagtaatattgtacatgggtgtttcatgtgctgttgtgtatttgctggtggtttgaatataaacactatgtatcagtgtcgtcccacagtgggtcntggggaggtttggctggggagcttaggacactaatccatcaggttggactcgaggtcctgcaccaactggcttggaaactagatgaggctgtcacagggctcagttgcataaaccgatggtgatggagtgtgggctgggtctttacactcattttattttttgtttctgcttttgttttcttcaatgatttgcaaggaaaccaaaagctggcagtgtttgtatgaacctgacagaacactgtcttcaaggaaatgcctcattcctgagaccagtaggtttgtttttttaggaagttccaatactaggaccccctacaagtactatggctcctcgaaaacacaaagttaatgccacaggaagcagcagatggtaggatgggatgcacaagagttcctgaaaactaacactgttagtgttttttttttaactcaatattttccatgaaaatgcaaccacatgtataatatttttaattaaataaaagtttcttgtgattgtt

SEQ ID NO: 22 - protein, 223 aa, Mus musculus
MACLGLRRYKAQLQLPSRTWPFVALLTLLFIPVFSEAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSDFLLWILVAVSLGLFFYSFLVSAVSLSKMLKKRSPLTTGVYVKMPPTEPECEKQFQPYFIPIN

SEQ ID NO: 23 - DNA, 672 bp, Homo sapiens
atggcttgccttggatttcagcggcacaaggctcagctgaacctggctgccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgacggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaattga

SEQ ID NO: 24 - protein, 223 aa, Homo sapiens
MACLGFQRHKAQLNLAARTWPCTLLFFLLFIPVFCKAMHVAQPAVVLASSRGIASFVCEYASPGKATEVRVTVLRQADSQVTEVCAATYMTGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 25 - DNA, 960 bp, Artificial Sequence
  Mus musculus CTLA4-Mus musculus IgG3
atggcttgtcttggactccggaggtacaaagctcaactgcagctgccttctaggacttggccttttgtagccctgctcactcttcttttcatcccagtcttctctgaagccatacaggtgacccaaccttcagtggtgttggctagcagccatggtgtcgccagctttccatgtgaatattcaccatcacacaacactgatgaggtccgggtgactgtgctgcggcagacaaatgaccaaatgactgaggtctgtgccacgacattcacagagaagaatacagtgggcttcctagattaccccttctgcagtggtacctttaatgaaagcagagtgaacctcaccatccaaggactgagagctgttgacacgggactgtacctctgcaaggtggaactcatgtacccaccgccatactttgtgggcatgggcaacgggacgcagatttatgtcattgatccagaaccatgcccggattctgatcaggagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacgcagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttc

SEQ ID NO: 26 - protein, 320 aa, Artificial Sequence
  Mus musculus CTLA4-Mus musculus IgG3
MACLGLRRYKAQLQLPSRTWPFVALLTLLFIPVFSEAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSDQEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNF

SEQ ID NO: 27 - DNA, 1004 bp, Homo sapiens
gcctcgctcgggcgcccagtggtcctgccgcctggtctcacctcgccatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtgaggctgcacccacccaggagtgtggccacgtgggcaaacaggcagttggccagagagcctggtgctgctgctgcaggggtgcaggcagaagcggggagctatgcccagtcagtgccagcccctc

SEQ ID NO: 28 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 29 - DNA, 1890 bp, Homo sapiens
aaacagagagagatagagaaagagaaagacagaggtgtttcccttagctatggaaactctataagagagatccagcttgcctcctcttgagcagtcagcaacagggtcccgtccttgacacctcagcctctacaggactgagaagaagtaaaaccgtttgctggggctggcctgactcaccagctgccatgcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaagagaagcactttgggattctttccattatgattctttgttacaggcaccgagaatgttgtattcagtgagggtcttcttacatgcatttgaggtcaagtaagaagacatgaaccaagtggaccttgagaccacagggttcaaaatgtctgtagctcctcaactcacctaatgtttatgagccagacaaatggaggaatatgacggaagaacatagaactctgggctgccatgtgaagagggagaagcatgaaaaagcagctacccaggtgttctacactcatcttagtgcctgagagtatttaggcagattgaaaaggacaccttttaactcacctctcaaggtgggccttgctacctcaagggggactgtctttcagatacatggttgtgacctgaggatttaagggatggaaaaggaagactagaggcttgcataataagctaaagaggctgaaagaggccaatgccccactggcagcatcttcacttctaaatgcatatcctgagccatcggtgaaactaacagataagcaagagagatgttttggggactcatttcattcctaacacagcatgtgtatttccagtgccaattgtaggggtgtgtgtgtgtgtgtgtgtgtgtgtgtatgactaaagagagaatgtagatattgtgaagtacatattaggaaaatatgggttgcatttggtcaagattttgaatgcttcctgacaatcaactctaatagtgcttaaaaatcattgattgtcagctactaatgatgttttcctataatataataaatatttatgtagatgtgcatttttgtgaaatgaaaacatgtaataaaaagtatatgttaggatacaaat

SEQ ID NO: 30 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 31 - DNA, 1803 bp, Homo sapiens
tgccaccttctctgccagaagataccatttcaactttaacacagcatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgaacagtgtcaccttgcaggctgtggtggagctgacgctgggagtcttcataatacagcacagcggttaagcccaccccctgttaactgcctatttataaccctaggatcctccttatggagaactatttattatacactccaaggcatgtagaactgtaataagtgaattacaggtcacatgaaaccaaaacgggccctgctccataagagcttatatatctgaagcagcaaccccactgatgcagacatccagagagtcctatgaaaagacaaggccattatgcacaggttgaattctgagtaaacagcagataacttgccaagttcagttttgtttctttgcgtgcagtgtctttccatggataatgcatttgatttatcagtgaagatgcagaagggaaatggggagcctcagctcacattcagttatggttgactctgggttcctatggccttgttggagggggccaggctctagaacgtctaacacagtggagaaccgaaaccccccccccccccccgccaccctctcggacagttattcattctctttcaatctctctctctccatctctctctttcagtctctctctctcaacctctttcttccaatctctctttctcaatctctctgtttccctttgtcagtctcttccctcccccagtctctcttctcaatccccctttctaacacacacacacacacacacacacacacacacacacacacacacacacacagagtcaggccgttgctagtcagttctcttctttccaccctgtccctatctctaccactatagatgagggtgaggagtagggagtgcagccctgagcctgcccactcctcattacgaaatgactgtatttaaaggaaatctattgtatctacctgcagtctccattgtttccagagtgaacttgtaattatcttgttatttattttttgaataataaagacctcttaacatt

SEQ ID NO: 32 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 33 - DNA, 1212 bp, Mus musculus
gcgcactggagctcatcgcagagcccagcgacaggcaggcgaccacagggggccacccgaggtggctggggccatggccggggtcgcgtgcttggggaaaactgcggatgccgatgaatggtgcgacagcggcctgggctctctaggtcccgacgcagcggctcccggaggaccaggtctgggcgcagagcttggcccagagctgtcgtgggcgcccttagtctttggctacgtcactgaggatggggacacagccctgcacttggctgtgattcatcagcatgagcccttcctggatttcctcctgggcttttccgccggccacgagtaccttgacctgcagaatgacctaggccaaacagccctgcatctagcagccatccttggggaggcatctacagtagagaagttgtatgcagccggtgcaggagtgttggtggctgagagagggggccacacggcattgcacttggcctgccgggtcagggcacacacgtgcgcgtgcgtactgctccagccccgtcccagccacccaagagatgcctcagatacctacctcactcagagccaggactgtaccccagacaccagccatgcccctgctgccgtggattcccaacccaacccagagaacgaagaggagccgcgtgatgaagactggaggctacaactagaagctgaaaactatgatggccataccccactccatgtagctgtcatccacaaagatgcagagatggtccggctgctcagggatgccggagccgacctcaataaaccggagcctacgtgtggccggacccctctgcacctggcagtagaagcccaggcagccagcgtgctggaacttctcctgaaagccggtgctgaccccaccgcccgcatgtatgggggccgcaccccgcttggcagtgccctgctccggcccaaccccatccttgcccgcctcctccgtgcacatggggcccctgaacctgaggacgaggacgataagcttagcccttgcagcagcagcggcagcgacagtgacagtgacaacagagatgagggcgatgaatatgatgacatcgtggttcacagtggcaggagccaaaaccgacaaccgccttccccggcatccaaacctcttcctgatgaccccaaccctgcctgacttaagtgctaatattaatataatttccaacttaataaaattgcagacctgacaaccag

SEQ ID NO: 34 - protein, 359 aa, Mus musculus
MAGVACLGKTADADEWCDSGLGSLGPDAAAPGGPGLGAELGPELSWAPLVFGYVTEDGDTALHLAVIHQHEPFLDFLLGFSAGHEYLDLQNDLGQTALHLAAILGEASTVEKLYAAGAGVLVAERGGHTALHLACRVRAHTCACVLLQPRPSHPRDASDTYLTQSQDCTPDTSHAPAAVDSQPNPENEEEPRDEDWRLQLEAENYDGHTPLHVAVIHKDAEMVRLLRDAGADLNKPEPTCGRTPLHLAVEAQAASVLELLLKAGADPTARMYGGRTPLGSALLRPNPILARLLRAHGAPEPEDEDDKLSPCSSSGSDSDSDNRDEGDEYDDIVVHSGRSQNRQPPSPASKPLPDDPNPA

SEQ ID NO: 35 - DNA, 1769 bp, Homo sapiens
cctcactgactataaaagaatagagaaggaagggcttcagtgaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaactgacctggaaagaaaaagcaataacctcaaagtgactattcagttttcaggatgatacactatgaagatgtttcaaaaaatctgaccaaaacaaacaaacagaaaacagaaaacaaaaaaacctctatgcaatctgagtagagcagccacaaccaaaaaattctacaacacacactgttctgaaagtgactcacttatcccaagaaaatgaaattgctgaaagatctttcaggactctacctcatatcagtttgctagcagaaatctagaagactgtcagcttccaaacattaatgcaatggttaacatcttctgtctttataatctactccttgtaaagactgtagaagaaagcgcaacaatccatctctcaagtagtgtatcacagtagtagcctccaggtttccttaagggacaacatccttaagtcaaaagagagaagaggcaccactaaaagatcgcagtttgcctggtgcagtggctcacacctgtaatcccaacattttgggaacccaaggtgggtagatcacgagatcaagagatcaagaccatagtgaccaacatagtgaaaccccatctctactgaaagtgcaaaaattagctgggtgtgttggcacatgcctgtagtcccagctacttgagaggctgaggcaggagaatcgtttgaacccgggaggcagaggttgcagtgtggtgagatcatgccactacactccagcctggcgacagagcgagacttggtttcaaaaaaaaaaaaaaaaaaaaacttcagtaagtacgtgttatttttttcaataaaattctattacagtatgtcaaaaaaaaaaaaaaaaaa

SEQ ID NO: 36 - protein, 281 aa, Homo sapiens [sequence omitted]

SEQ ID NO: 37 - DNA, 4150 bp, Homo sapiens
tccggccccagagaactcagcctcattcctgctttaaaatctctcggccacctttgatgaggggactgggcagttctagacagtcccgaagttctcaaggcacaggtctcttcctggtttgactgtccttaccccggggaggcagtgcagccagctgcaagccccacagtgaagaacatctgagctcaaatccagataagtgacataagtgacctgctttgtaaagccatagagatggcctgtccttggaaatttctgttcaagaccaaattccaccagtatgcaatgaatggggaaaaagacatcaacaacaatgtggagaaagccccctgtgccacctccagtccagtgacacaggatgaccttcagtatcacaacctcagcaagcagcagaatgagtccccgcagcccctcgtggagacgggaaagaagtctccagaatctctggtcaagctggatgcaaccccattgtcctccccacggcatgtgaggatcaaaaactggggcagcgggatgactttccaagacacacttcaccataaggccaaagggattttaacttgcaggtccaaatcttgcctggggtccattatgactcccaaaagtttgaccagaggacccagggacaagcctacccctccagatgagcttctacctcaagctatcgaatttgtcaaccaatattacggctccttcaaagaggcaaaaatagaggaacatctggccagggtggaagcggtaacaaaggagatagaaacaacaggaacctaccaactgacgggagatgagctcatcttcgccaccaagcaggcctggcgcaatgccccacgctgcattgggaggatccagtggtccaacctgcaggtcttcgatgcccgcagctgttccactgcccgggaaatgtttgaacacatctgcagacacgtgcgttactccaccaacaatggcaacatcaggtcggccatcaccgtgttcccccagcggagtgatggcaagcacgacttccgggtgtggaatgctcagctcatccactatgctggctaccagatgccagatggcagcatcagaggggaccctgccaacgtggaattcactcagctgtgcatcgacctgggctggaagcccaagtacggccgcttcgatgtggtccccctggtcctgcaggccaatggccgtgaccctgagctcttcgaaatcccacctgaccttgtgcttgaggtggccatggaacatcccaaatacgagtggtttcgggaactggagctaaagtggtacgccctgcctgcagtggccaacatgctgcttgaggtgggcggcctggagttcccagggtgccccttcaatggctggtacatgggcacagagatcggagtccgggacttctgtgacgtccagcgctacaacatcctggaggaagtgggcaggagaatgggcctggaaacgcacaagctggcctcgctctggaaagaccaggctgtcgttgagatcaacattgctgtgctccatagttttcagaagcagaatgtgaccatcatggaccaccactcgactgcagaatccttcatgaagtacatgcagaatgaataccggtcccgtgggggctgcccggcagactggatttggctggtccctcccatgtctgggagcatcacccccgtgtttcaccaggagatgctgaactacgtcctgtcccctttctactactatcaggtagaggcctggaaaacccatgtctggcaggacgagaagcggagacccaagagaagagagattccattgaaagtcttggtcaaagctgtgctctttgcctgtatgctgatgcgcaagacaatggcgtcccgagtcagagtcaccatcctctttgctacagagacaggaaaatcagaggcgctggcctgggacctgggggccttattcagctgtgccttcaaccccaaggttgtctgcatggataagtacaggctgagctgcctggaggaggaacggctgctgttggtggtgaccagtacgtttggcaatggagactgccctggcaatggagagaaactgaagaaatcgctcttcatgctgaaagagctcaacaacaaattcaggtacgctgtgtttggcctcggctccagcatgtaccctcggttctgcgcctttgctcatgacattgatcagaagctgtcccacctgggggcctctcagctcaccccgatgggagaaggggatgagctcagtgggcaggaggacgccttccgcagctgggccgtgcaaaccttcaaggcagcctgtgagacgtttgatgtccgaggcaaacagcacattcagatccccaagctctacacctccaatgtgacctgggacccgcaccactacaggctcgtgcaggactcacagcctttggacctcagcaaagccctcagcagcatgcatgccaagaacgtgttcaccatgaggctcaaatctcggcagaatctacaaagtccgacatccagccgtgccaccatcctggtggaactctcctgtgaggatggccaaggcctgaactacctgccgggggagcaccttggggtttgtccaggcaaccagccggccctggtccaaggtatcctggagcgagtggtggatggccccacaccccaccagacagtgcgcctggaggccctggatgagagtggcagctactgggtcagtgacaagaggctgcccccctgctcactcagccaggccctcacctacttcctggacatcaccacacccccaacccagctgctgctccaaaagctggcccaggtggccacagaagagcctgagagacagaggctggaggccctgtgccagccctcagagtacagcaagtggaagttcaccaacagccccacattcctggaggtgctagaggagttcccgtccctgcgggtgtctgctggcttcctgctttcccagctccccattctgaagcccaggttctactccatcagctcctcccgggatcacacgcccacagagatccacctgactgtggccgtggtcacctaccacacccgagatggccagggtcccctgcaccacggcgtctgcagcacatggctcaacagcctgaagccccaagacccagtgccctgctttgtgcggaatgccagcggcttccacctccccgaggatccctcccatccttgcatcctcatcgggcctggcacaggcatcgcgcccttccgcagtttctggcagcaacggctccatgactcccagcacaagggagtgcggggaggccgcatgaccttggtgtttgggtgccgccgcccagatgaggaccacatctaccaggaggagatgctggagatggcccagaagggggtgctgcatgcggtgcacacagcctattcccgcctgcctggcaagcccaaggtctatgttcaggacatcctgcggcagcagctggccagcgaggtgctccgtgtgctccacaaggagccaggccacctctatgtttgcggggatgtgcgcatggcccgggacgtggcccacaccctgaagcagctggtggctgccaagctgaaattgaatgaggagcaggtcgaggactatttctttcagctcaagagccagaagcgctatcacgaagatatctttggtgctgtatttccttacgaggcgaagaaggacagggtggcggtgcagcccagcagcctggagatgtcagcgctctgagggcctacaggaggggttaaagctgccggcacagaacttaaggatggagccagctctgcattatctgaggtcacagggcctggggagatggaggaaagtgatatcccccagcctcaagtcttatttcctcaacgttgctccccatcaagccctttacttgacctcctaacaagtagcaccctggattgatcggagcctcctctctcaaactggggcctccctggtcccttggagacaaaatcttaaatgccaggcctggcgagtgggtgaaagatggaacttgctgctgagtgcaccacttcaagtgaccaccaggaggtgctatcgcaccactgtgtatttaactgccttgtgtacagttatttatgcctctgtatttaaaaaactaacacccagtctgttccccatggccacttgggtcttccctgtatgattccttgatggagatatttac

SEQ ID NO: 38 - protein, 1153 aa, Homo sapiens [sequence omitted]

SEQ ID NO: 39 - DNA, 2943 bp, Homo sapiens
accaggcaacaccattgaaggctcatatgtaaaaatccatgccttcctttctcccaatctccattcccaaacttagccactggcttctggctgaggccttacgcatacctcccggggcttgcacacaccttcttctacagaagacacaccttgggcatatcctacagaagaccaggcttctctctggtccttggtagagggctactttactgtaacagggccagggtggagagttctctcctgaagctccatcccctctataggaaatgtgttgacaatattcagaagagtaagaggatcaagacttctttgtgctcaaataccactgttctcttctctaccctgccctaaccaggagcttgtcaccccaaactctgaggtgatttatgccttaatcaagcaaacttccctcttcagaaaagatggctcattttccctcaaaagttgccaggagctgccaagtattctgccaattcaccctggagcacaatcaacaaattcagccagaacacaactacagctactattagaactattattattaataaattcctctccaaatctagccccttgacttcggatttcacgatttctcccttcctcctagaaacttgataagtttcccgcgcttccctttttctaagactacatgtttgtcatcttataaagcaaaggggtgaataaatgaaccaaatcaataacttctggaatatctgcaaacaacaataatatcagctatgccatctttcactattttagccagtatcgagttgaatgaacatagaaaaatacaaaactgaattcttccctgtaaattccccgttttgacgacgcacttgtagccacgtagccacgcctacttaagacaattacaaaaggcgaagaagactgactcaggcttaagctgccagccagagagggagtcatttcattggcgtttgagtcagcaaagaagtcaagatggccaaagttccagacatgtttgaagacctgaagaactgttacagtgaaaatgaagaagacagttcctccattgatcatctgtctctgaatcagaaatccttctatcatgtaagctatggcccactccatgaaggctgcatggatcaatctgtgtctctgagtatctctgaaacctctaaaacatccaagcttaccttcaaggagagcatggtggtagtagcaaccaacgggaaggttctgaagaagagacggttgagtttaagccaatccatcactgatgatgacctggaggccatcgccaatgactcagaggaagaaatcatcaagcctaggtcagcaccttttagcttcctgagcaatgtgaaatacaactttatgaggatcatcaaatacgaattcatcctgaatgacgccctcaatcaaagtataattcgagccaatgatcagtacctcacggctgctgcattacataatctggatgaagcagtgaaatttgacatgggtgcttataagtcatcaaaggatgatgctaaaattaccgtgattctaagaatctcaaaaactcaattgtatgtgactgcccaagatgaagaccaaccagtgctgctgaaggagatgcctgagatacccaaaaccatcacaggtagtgagaccaacctcctcttcttctgggaaactcacggcactaagaactatttcacatcagttgcccatccaaacttgtttattgccacaaagcaagactactgggtgtgcttggcaggggggccaccctctatcactgactttcagatactggaaaaccaggcgtaggtctggagtctcacttgtctcacttgtgcagtgttgacagttcatatgtaccatgtacatgaagaagctaaatcctttactgttagtcatttgctgagcatgtactgagccttgtaattctaaatgaatgtttacactctttgtaagagtggaaccaacactaacatataatgttgttatttaaagaacaccctatattttgcatagtaccaatcattttaattattattcttcataacaattttaggaggaccagagctactgactatggctaccaaaaagactctacccatattacagatgggcaaattaaggcataagaaaactaagaaatatgcacaatagcagttgaaacaagaagccacagacctaggatttcatgatttcatttcaactgtttgccttctacttttaagttgctgatgaactcttaatcaaatagcataagtttctgggacctcagttttatcattttcaaaatggagggaataatacctaagccttcctgccgcaacagttttttatgctaatcagggaggtcattttggtaaaatacttcttgaagccgagcctcaagatgaaggcaaagcacgaaatgttattttttaattattatttatatatgtatttataaatatatttaagataattataatatactatatttatgggaaccccttcatcctctgagtgtgaccaggcatcctccacaatagcagacagtgttttctgggataagtaagtttgatttcattaatacagggcattttggtccaagttgtgcttatcccatagccaggaaactctgcattctagtacttgggagacctgtaatcatataataaatgtacattaattaccttgagccagtaattggtccgatctttgactcttttgccattaaacttacctgggcattcttgtttcaattccacctgcaatcaagtcctacaagctaaaattagatgaactcaactttgacaaccatgagaccactgttatcaaaactttcttttctggaatgtaatcaatgtttcttctaggttctaaaaattgtgatcagaccataatgttacattattatcaacaatagtgattgatagagtgttatcagtcataactaaataaagcttgcaacaaaattctctgacaaaaaaaaaaaaaaaa

SEQ ID NO: 40 - protein, 271 aa, Homo sapiens
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 41 - DNA, 1358 bp, Homo sapiens
aaaagccatggccgacaaggtcctgaaggagaagagaaagctgtttatccgttccatgggtgaaggtacaataaatggcttactggatgaattattacagacaagggtgctgaacaaggaagagatggagaaagtaaaacgtgaaaatgctacagttatggataagacccgagctttgattgactccgttattccgaaaggggcacaggcatgccaaatttgcatcacatacatttgtgaagaagacagttacctggcagggacgctgggactctcagcagatcaaacatctggaaattaccttaatatgcaagactctcaaggagtactttcttcctttccagctcctcaggcagtgcaggacaacccagctatgcccacatcctcaggctcagaagggaatgtcaagctttgctccctagaagaagctcaaaggatatggaaacaaaagtcggcagagatttatccaataatggacaagtcaagccgcacacgtcttgctctcattatctgcaatgaagaatttgacagtattcctagaagaactggagctgaggttgacatcacaggcatgacaatgctgctacaaaatctggggtacagcgtagatgtgaaaaaaaatctcactgcttcggacatgactacagagctggaggcatttgcacaccgcccagagcacaagacctctgacagcacgttcctggtgttcatgtctcatggtattcgggaaggcatttgtgggaagaaacactctgagcaagtcccagatatactacaactcaatgcaatctttaacatgttgaataccaagaactgcccaagtttgaaggacaaaccgaaggtgatcatcatccaggcctgccgtggtgacagccctggtgtggtgtggtttaaagattcagtaggagtttctggaaacctatctttaccaactacagaagagtttgaggatgatgctattaagaaagcccacatagagaaggattttatcgctttctgctcttccacaccagataatgtttcttggagacatcccacaatgggctctgtttttattggaagactcattgaacatatgcaagaatatgcctgttcctgtgatgtggaggaaattttccgcaaggttcgattttcatttgagcagccagatggtagagcgcagatgcccaccactgaaagagtgactttgacaagatgtttctacctcttcccaggacattaaaataaggaaactgtatgaatgtctgtgggcagggtgaagagatccttctgtaaaggtttttgaattatgtctgctgaataataaacttttttgaaataataaatctggtagaaaaatgaaaaaaaaaaaaaaaaaa

SEQ ID NO: 42 - protein, 404 aa, Homo sapiens
MADKVLKEKRKLFIRSMGEGTINGLLDELLQTRVLNKEEMEKVKRENATVMDKTRALIDSVIPKGAQACQICITYICEEDSYLAGTLGLSADQTSGNYLNMQDSQGVLSSFPAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKDSVGVSGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGH

SEQ ID NO: 43 - DNA, 6752 bp, Homo sapiens
ctgaatagtgtccaggtgatttagaggataaataaaaatccattaaagaggtaaagacataaaaacgagaaacatggactggtttacacataacacatacaaagtctattataaaactagcatcagtatccttgaatgcaaacctttttctgagtatttaacaatcgcaccctttaaaaaatgtacaatagacattaagagacttaaacagatatataatcattttaaattaaaatagcgttaaacagtacctcaagctcaataagcattttaagtattctaatcttagtatttctctagctgacatgtaagaagcaatctatcttattgtatgcaattagctcattgtgtggataaaaaggtaaaaccattctgaaacaggaaaccaatacacttcctgtttaatcaacaaatctaaacatttattcttttcatctgtttactcttgctcttgtccaccacaatatgctattcacatgttcagtgtagttttatgacaaagaaaattttctgagttacttttgtatccccacccccttaaagaaaggaggaaaaactgtttcatacagaaggcgttaattgcatgaattagagctatcacctaagtgtgggctaatgtaacaaagagggatttcacctacatccattcagtcagtctttgggggtttaaagaaattccaaagagtcatcagaagaggaaaaatgaaggtaatgttttttcagacaggtaaagtctttgaaaatatgtgtaatatgtaaaacattttgacacccccataatatttttccagaattaacagtataaattgcatctcttgttcaagagttccctatcactctctttaatcactactcacagtaacctcaactcctgccacaatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaatgtaagtatatttcctttcttactaaaattattacatttagtaatctagctggagatcatttcttaataacaatgcattatactttcttagaattacaagaatcccaaactcaccaggatgctcacatttaagttttacatgcccaagaaggtaagtacaatattttatgttcaatttctgttttaataaaattcaaagtaatatgaaaatttgcacagatgggactaatagcagctcatctgaggtaaagagtaactttaatttgtttttttgaaaacccaagtttgataatgaagcctctattaaaacagttttacctatatttttaatatatatttgtgtgttggtgggggtgggaagaaaacataaaaataatattctcactttatcgataagacaattctaaacaaaaatgttcatttatggtttcatttaaaaatgtaaaactctaaaatatttgattatgtcattttagtatgtaaaataccaaaatctatttccaaggagcccacttttaaaaatcttttcttgttttaggaaaggtttctaagtgagaggcagcataacactaatagcacagagtctggggccagatatctgaagtgaaatctcagctctgccatgtcctagctttcatgatctttggcaaattacctactctgtttgtgattcagtttcatgtctacttaaatgaataactgtatatacttaatatggctttgtgagaattagtaagtaaatgtaaagcactcagaaccgtgtctggcataaggtaaataccatacaagcattagctattattagtagtattaaagataaaattttcactgagaaatacaaagtaaaattttggactttatctttttaccaatagaacttgagatttataatgctatatgacttattttccaagattaaaagcttcattaggttgtttttggattcagatagagcataagcataatcatccaagctcctaggctacattaggtgtgtaaagctacctagtagctgtgccagttaagagagaatgaacaaaatctggtgccagaaagagcttgtgccagggtgaatccaagcccagaaaataataggatttaaggggacacagatgcaatcccattgactcaaattctattaattcaagagaaatctgcttctaactacccttctgaaagatgtaaaggagacagcttacagatgttactctagtttaatcagagccacataatgcaactccagcaacataaagatactagatgctgttttctgaagaaaatttctccacattgttcatgccaaaaacttaaacccgaatttgtagaatttgtagtggtgaattgaaagcgcaatagatggacatatcaggggattggtattgtcttgacctacctttcccactaaagagtgttagaaagatgagattatgtgcataatttagggggtggtagaattcatggaaatctaagtttgaaaccaaaagtaatgataaactctattcatttgttcatttaaccctcattgcacatttacaaaagattttagaaactaataaaaatatttgattccaaggatgctatgttaatgctataatgagaaagaaatgaaatctaattctggctctacctacttatgtggtcaaattctgagatttagtgtgcttatttataaagtggagatgatacttcactgcctacttcaaaagatgactgtgagaagtaaatgggcctattttggagaaaattcttttaaattgtaatataccatagaaatatgaaatattatatataatatagaatcaagaggcctgtccaaaagtcctcccaaagtattataatcttttatttcactgggacaaacatttttaaaatgcatcttaatgtagtgattgtagaaaagtaaaaatttaagacatatttaaaaatgtgtcttgctcaaggctatattgagagccactactacatgattattgttacctagtgtaaaatgttgggattgtgatagatggcatccaagagttccttctctctcaacattctgtgattcttaactcttagactatcaaatattataatcatagaatgtgatttttatgcttccacattctaactcatctggttctaatgattttctatgcagattggaaaagtaatcagcctacatctgtaataggcatttagatgcagaaagtctaacattttgcaaagccaaattaagctaaaaccagtgagtcaactatcacttaacgctagtcataggtacttgagccctagtttttccagttttataatgtaaactctactggtccatctttacagtgacattgagaacagagagaatggtaaaaactacatactgctactccaaataaaataaattggaaattaatttctgattctgacctctatgtaaactgagctgatgataattattattctaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaaggtaaggcattactttatttgctctcctggaaataaaaaaaaaaaagtagggggaaaagtaccccattttaaagtgacataacatttttggtatttgtaaagtacccatgcatgtaattagcctacattttaagtacactgtgaacatgaatcatttctaatgttaaatgattaactggggagtataagctactgagtttgcacctaccatctactaatggacaagcctcatcccaaactccatcacctttcatattaacacaaaactgggagtgagagaaggtactgagttgagtttcacagaaagcaggcagattttattatatatttttcaattccttcagatcatttactggaatagccaatactgattacctgaaaggcttttcaaatggtgtttccttatcatttgatggaaggactacccataagagatttgtcttaaaaaaaaaaactggagccattaaaatggccagtggactaaacaaacaacaatctttttagaggcaatccccactttcagaatcttaagtatttttaaatgcacaggaagcataaaatatgcaagggactcaggtgatgtaaaagagattcacttttgtctttttatatcccgtctcctaaggtataaaattcatgagttaataggtatcctaaataagcagcataagtatagtagtaaaagacattcctaaaagtaactccagttgtgtccaaatgaatcacttattagtggactgtttcagttgaattaaaaaaatacattgagatcaatgtcatctagacattgacagattcagttccttatctatggcaagagttttactctaaaataattaacatcagaaaactcattcttaactcttgatacaaatttaagacaaaaccatgcaaaaatctgaaaactgtgtttcaaaagccaaacactttttaaaataaaaaaatcccaagatatgacaatatttaaacaattatgcttaagaggatacagaacactgcaacagttttttaaaagagaatacttatttaaagggaacactctatctcacctgcttttgttcccagggtaggaatcacttcaaatttgaaaagctctcttttaaatctcactatatatcaaaaatatttcctccttagcttatcaactagaggaagcgtttaaatagctcctttcagcagagaagcctaatttctaaaaagccagtccacagaacaaaatttctaatgtttaaacttttaaaagttggcaaattcacctgcattgatactatgatggggtagggataggtgtaagtatttatgaagatgttcttcacacaaatttatcccaaacagaagcatgtcctagcttactctagtgtagttctgttctgctttggggaaaatataaggagattcacttaagtagaaaaataggagactctaatcaagatttagaaaagaagaaagtataatgtgcatatcaattcatacatttaacttacacaaatataggtgtacattcagaggaaaagcgatcaagtttatttcacatccagcatttaatatttgtctagatctatttttatttaaatctttatttgcacccaatttagggaaaaaatttttgtgttcattgactgaattaacaaatgaggaaaatctcagcttctgtgttactatcatttggtatcataacaaaatatgtaattttggcattcattttgatcatttcaagaaaatgtgaataattaatatgtttggtaagcttgaaaataaaggcaacaggcctataagacttcaattgggaataactgtatataaggtaaactactctgtactttaaaaaattaacatttttcttttatagggatctgaaacaacattcatgtgtgaatatgctgatgagacagcaaccattgtagaatttctgaacagatggattaccttttgtcaaagcatcatctcaacactgacttgataattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaacaagcctggatatttgttattttggaaacagcacagagtaagcatttaaatatttcttagttacttgtgtgaactgtaggatggttaaaatgcttacaaaagtcactctttctctgaagaaatatgtagaacagagatgtagacttctcaaaagcccttgctttgtcctttcaagggctgatcagacccttagttctggcatctcttagcagattatattttccttcttcttaaaatgccaaacacaaacactcttgaaactcttcatagatttggtgtggctatgaattctccaatatcttacaccctgcccagtgctgtgaggaggctcacctgtatggcctatatcaaaggtcttccctgccctttggctttccattgggtcctgccactggggagtgctggtaggaactatgaggaacataagagattcccttgactccctccttgtggagtagacccaggatggctgtgtctctcaagcaaggaacccagattacctcaaggtggcactctgggtactttttccttctgagtgattctggtaatcttcccttgtccctttaagcctagggagggtggtacttttgctgttagcaactccagggtacttgtaccatcccttgcagtttccctgaactctgaccatagctttttaaatagtccttttattaaatcctccttttgattgagtatgccatctatttcctgctgggactcagatacagtaattgtatcagaaatagccccagaaaatagaccctcaaaataggattctgggactgggttgttcatatattcaaggaatgcaaggataataggacatgggaaatctacgggatgtagtagcatcgcaattactgaacttatcatcaatggtagaatgggatgaaatgcagac

SEQ ID NO: 44 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 45 - DNA, 924 bp, Homo sapiens
cagagccccacgaaggaccagaacaagacagagtgcctcctgccgatccaaacatgagccgcctgcccgtcctgctcctgctccaactcctggtccgccccggactccaagctcccatgacccagacaacgcccttgaagacaagctgggttaactgctctaacatgatcgatgaaattataacacacttaaagcagccacctttgcctttgctggacttcaacaacctcaatggggaagaccaagacattctgatggaaaataaccttcgaaggccaaacctggaggcattcaacagggctgtcaagagtttacagaacgcatcagcaattgagagcattcttaaaaatctcctgccatgtctgcccctggccacggccgcacccacgcgacatccaatccatatcaaggacggtgactggaatgaattccggaggaaactgacgttctatctgaaaacccttgagaatgcgcaggctcaacagacgactttgagcctcgcgatcttttgagtccaacgtccagctcgttctctgggccttctcaccacagagcctcgggacatcaaaaacagcagaacttctgaaacctctgggtcatctctcacacattccaggaccagaagcatttcaccttttcctgcggcatcagatgaattgttaattatctaatttctgaaatgtgcagctcccatttggccttgtgcggttgtgttctcatttttatcccattgagactatttatttatgtatgtatgtatttatttatttattgcctggagtgtgaactgtatttattttagcagaggagccatgtcctgctgcttctgcaaaaaactcagagtggggtggggagcatgttcatttgtacctcgagttttaaactggttcctagggatgtgtgagaataaactagactctgaac

SEQ ID NO: 46 - protein, 152 aa, Homo sapiens
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 47 - DNA, 921 bp, Homo sapiens
ttctatgcaaagcaaaaagccagcagcagccccaagctgataagattaatctaaagagcaaattatggtgtaatttcctatgctgaaactttgtagttaattttttaaaaaggtttcattttcctattggtctgatttcacaggaacattttacctgtttgtgaggcattttttctcctggaagagaggtgctgattggccccaagtgactgacaatctggtgtaacgaaaatttccaatgtaaactcattttccctcggtttcagcaattttaaatctatatatagagatatctttgtcagcattgcatcgttagcttctcctgataaactaattgcctcacattgtcactgcaaatcgacacctattaatgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctgaatattttaatttatgagtttttgatagctttattttttaagtatttatatatttataactcatcataaaataaagtatatatagaatct

SEQ ID NO: 48 - protein, 153 aa, Homo sapiens [sequence omitted]

SEQ ID NO: 49 - DNA, 1125 bp, Homo sapiens
ttctgccctcgagcccaccgggaacgaaagagaagctctatctcgcctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattc

SEQ ID NO: 50 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 51 - DNA, 5191 bp, Homo sapiens
gaattcagtaacccaggcattattttatcctcaagtcttaggttggttggagaaagataacaaaaagaaacatgattgtgcagaaacagacaaacctttttggaaagcatttgaaaatggcattccccctccacagtgtgttcacagtgtgggcaaattcactgctctgtcgtactttctgaaaatgaagaactgttacaccaaggtgaattatttataaattatgtacttgcccagaagcgaacagacttttactatcataagaacccttccttggtgtgctctttatctacagaatccaagacctttcaagaaaggtcttggattcttttcttcaggacactaggacataaagccacctttttatgatttgttgaaatttctcactccatcccttttgctgatgatcatgggtcctcagaggtcagacttggtgtccttggataaagagcatgaagcaacagtggctgaaccagagttggaacccagatgctctttccactaagcatacaactttccattagataacacctccctcccaccccaaccaagcagctccagtgcaccactttctggagcataaacataccttaactttacaacttgagtggccttgaatactgttcctatctggaatgtgctgttctctttcatcttcctctattgaagccctcctattcctcaatgccttgctccaactgcctttggaagattctgctcttatgcctccactggaattaatgtcttagtaccacttgtctattctgctatatagtcagtccttacattgctttcttcttctgatagaccaaactctttaaggacaagtacctagtcttatctatttctagatcccccacattactcagaaagttactccataaatgtttgtggaactgatttctatgtgaagacatgtgccccttcactctgttaactagcattagaaaaacaaatcttttgaaaagttgtagtatgcccctaagagcagtaacagttcctagaaactctctaaaatgcttagaaaaagatttattttaaattacctccccaataaaatgattggctggcttatcttcaccatcatgatagcatctgtaattaactgaaaaaaaataattatgccattaaaagaaaatcatccatgatcttgttctaacacctgccactctagtactatatctgtcacatggtctatgataaagttatctagaaataaaaaagcatacaattgataattcaccaaattgtggagcttcagtattttaaatgtatattaaaattaaattattttaaagatcaaagaaaactttcgtcatactccgtatttgataaggaacaaataggaagtgtgatgactcaggtttgccctgaggggatgggccatcagttgcaaatcgtggaatttcctctgacataatgaaaagatgagggtgcataagttctctagtagggtgatgatataaaaagccaccggagcactccataaggcacaaactttcagagacagcagagcacacaagcttctaggacaagagccaggaagaaaccaccggaaggaaccattctcactgtgtgtaaacatgacttccaagctggccgtggctctcttggcagccttcctgatttctgcagctctgtgtgaaggtaagcacatctttctgacctacagcgttttcctatgtctaaatgtgatccttagatagcaaagctattcttgatgctttggtaacaaacatcctttttattcagaaacagaatataatcttagcagtcaattaatgttaaattgaagatttagaaaaaactatatataacacttaggaaatataaaggtttgatcaatatagatattctgcttttataatttataccaggtagcatgcatatatttaacgtaaataagtaatttatagtatgtcctattgagaaccacggttacctatattatgtattaatattgagttgagcaaggtaactcagacaattccactccttgtagtatttcattgacaagcctcagatttgtcattaattcctgtctggtttaaagataccctgattatagaccaggcatgtataacttatttatatatttctgttaattctttctgaaggcaatttctatgctggagagtcttagcttgcctactataaataacactgtggtatcacagaggattatgcaatattgaccagataaaaataccatgaagatgttgatattgtacaaaaagaactctaactcttatataggaagttgttcaatgttgtcagttatgactgttttttaaaacaaagaactaactgaggtcaagggctaggagatattcaggaatgagttcactagaaacatgatgccttccatagtctccaaataatcatattggaattagaaggaagtagctggcagagctgtgcctgttgataaaatcaatccttaatcactttttcccccaacaggtgcagttttgccaaggagtgctaaagaacttagatgtcagtgcataaagacatactccaaacctttccaccccaaatttatcaaagaactgagagtgattgagagtggaccacactgcgccaacacagaaattatgtaagtactttaaaaaagattagatattttgttttagcaaacttaaaattaaggaaggtggaaatatttaggaaagttccaggtgttaggattacagtagtaaatgaaacaaaacaaaataaaaatatttgtctacatgacatttaaatatggtagcttccacaactactataaatgttattttggacttagactttatgcctgacttaaggaatcatgatttgaatgcaaaaactaaatattaatctgaaccatttctttcttatttcagtgtaaagctttctgatggaagagagctctgtctggaccccaaggaaaactgggtgcagagggttgtggagaagtttttgaagaggtaagttatatattttttaatttaaatttttcatttatcctgagacatataatccaaagtcagcctataaatttctttctgttgctaaaaatcgtcattaggtatctgcctttttggttaaaaaaaaaggaatagcatcaatagtgagtttgttgtacttatgaccagaaagaccatacatagtttgcccaggaaattctgggtttaagcttgtgtcctatactcttagtaaagttctttgtcactcccagtagtgtcctattttagatgataatttctttgatctccctatttatagttgagaatatagagcatttctaacacatgaatgtcaaagactatattgacttttcaagaaccctactttccttcttattaaacatagctcatctttatatttttaattttattttagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagatatacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttagtgtttttatgtgctctccaaattttttttactgtttctgattgtatggaaatataaaagtaaatatgaaacatttaaaatataatttgttgtcaaagtaatcaagtgtttgtcttttttttagttttagcttattgggattctctttgtttatatttaaaattatactttgatttagaaaacataaatgcttccccttagcattttgttatggaaaattacaaacttttatttttagaaaacagaactcctttccagaaataggttacaaacagtagtgtcctccacagaatgttggaaatgttttcaactccccactgtatactatcttgctaataagtctgtcttcagatttcgattaaccggtttgtatgtctgtgcactttagcatagctggacattaaagaggaaagagagtacatattataagttgcttatcagtaactgaggagtaaaactgataaatgtgaggcaaagaagtttaaaatatggttaaagcctaagcatatttgcaaacaaatcaaacaatactctgagaagtaaaaacataattatttaattaacaaatttcagtggataaattttataacaaattagacacagttgaaaataaaattagaaaactagaaaatagaacaaaagaaacttctggaattca

SEQ ID NO: 52 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 53 - DNA, 4663 bp, Homo sapiens
cccgggaggcagaggttgcagcgaacctggattgtgccaccgcactccagcctggtgacagagggagactctgtctcaaaaaaaaaaaaaaaaaaaaaagaaatgttactagtatgtagtaagttctcagtaaatgttagctactatactctttcaagtgctgggtttttacttgatgtcatacagtgttatataagatctccaaagatactgaggagtcctcaaggccaattttaacaagcatggttgccgcattcttgtgcttatagttgaacatttcttctttcagacacttgcacaaagtgatacttctaagatgcatttgcattaggtggcaaacttcatcctgggtatgaaaaacattgagatttgggaataaagcatagtaagactgaggttgcaattactaaaggaaaaccccaacagagataagtgaagttctgcaatatcatgcaccctcccccaacccgctctgtctccccaggccccccttcgttagaacacccatgactggctatattatatcagcatttcccataatgtaaaaagggaaaatacagacctgggcgttcatggaaagtattctaactctcacaaccagaatccctgtctttgaattttttttcttggtttttagatctttaacttttccttcagcatttcagtactcaactttttgaaaatcatcttttctgaggaatgatatttcctggcacagcatcatctctgtcaagtgactcagtttgatttttttgtttgttagtataaagtggccccaacttacagagaaaaagtgggctcttggtatcagtttgatgtcagggtttttccgtgtttgagagggagctttaaataccactcgatttgaaggtgtctgcaagcgagctccagtccgctgtcaagatgcttctggccatggtccttacctctgccctgctcctgtgctccgtggcaggccaggggtgtccaaccttggcggggatcctggacatcaacttcctcatcaacaagatgcaggtaggctgcagggggagcccatgggaaagacagctactgacaaagtgaaatatgtatgaggatgaaaaaactcggggctgactaaaggttcttatctctctatctactttaggaagatccagcttccaagtgccactgcagtgctaatgtgagtgaatgctctttaagaactttccaaattaattttaattttcacatctggaatcttcactctgaaatttcccttgcaggtgaccagttgtctctgtttgggcattccctctgtaagtatagtgaaataacataatgttgaccttggatttttttggtttgtttttaagtaaaaataagttgctttatttaatatttaatgttatacattgttgcttaatttaattgttacagattagtattccctgttaaaaccacattgttacaaattattcccttttaaaactacgatcttgaaatcctatattatgaacatttctttgtatttaattaactttatgcctcttgagaagtttgaacacttttcaacattaaaaaaagaatcctgaatatctttttagataggtggccatgtgcacaattaaataaaactggaactaaggatataataattgctgtagctcatatcatattgctttctaactcatttactgataactctagagttgtgaaacaatgtaaataaaatgacaactccttatctttcatctgtcatgaatgatctatgcgctatacctccccctccctgcctcctcccttcctccccaccaccctgttgtctgtctagctgattagagtgactgttggtttgaatgctgccctctgggcaggtagaggatctgaggttgtgagtggaaggagggcttccagagggccactgcccactacggcaggaaggatgggtggcaggaaagttctgattcctaattcaaactcctggttagggtgaggaggaggcacttctccaaggtgcagtgctttattctttctcatgcaaggcctgggagaatctgaagaatctgagcttcttgccctggctagggtaagacatcgcacccatcgcggtccatccattagatgagaagaggatagagtgccttctgggcaggaaccaggcagacagcacagcccctgtcccttggagtaccgtccatgtttttagctgctgctgaaataccagctgcattcaattgtcacatcccattagctggtgtgaaaaggcttttcctcactctgcactttcagacttacaagccttgaagccgggaagcacccgttgaaaagaacattcagagccgactatttcagggcccagagccctcatgtttcctggatgtaacatacaggaagtctcctccaggggatgtcactgtggaaaaatggcatcccctttaaatacgggagatcacttcctacattggcaagggacctgtctaaaaataatgcaagtttgagtaatggtgattaaataaaaatcatctctattatattgctctttgtgatatatttccaaagctgtcctcagaatatttctttgaataaatccttactatttaccaggacaactgcaccagaccatgcttcagtgagagactgtctcagatgaccaataccaccatgcaaacaagatacccactgattttcagtcgggtgaaaaaatcagttgaagtactaaagaacaacaagtgtccagtaagtttgttttcatatgtgatatgttcctgttggtgatttctatgtgaatggtgatgccaaccctgtttgaacacaaaaggatgataaagttggaattggtagttcaaggttgataaaagacatctaagaattttaatcagaagtaatataattaaagtgagatccactgaaacaatagaattaaagtgagatagatcattgttcctgacgaggccatttacttctctctactatggaataatgaaagaatcctttctgagtgtaattagaagctacaatctagagaatcagggatgtagctcacataatactaaattatcctagagattcaatgtactaactgaatggatgttgttaacagggatttttttttcctgttggttaaggaggttttgttttgttttggagacagagtcttgctctgttgcccaggctggagtgcagtggtgccatctgagctcactgcagcctctgcctcccgggttcaagtgattatcctgcctcagcctcccgagtagctggcattacaggtgcgtgccaccatgcctggctaatttttgtatttttaatagagatggggtttcaccatgttggccaggttgctctccaactcctgaactcaagtgatttgcccgccttgacctcccaaagtgctgggatgacaggtgtgagccaccatgcctggcctgcattaaggaggtatttaaagggcaatgcacccaggtcaaggtggaagcttgctactcatcctgaatgcccatccacacattcttttcttcagcatataccctagtccctgacagcagactgggatggcaagttgggtagaggtgacctccctgtgttttttgggtattagcatctccacacaagatcctagaaggctgaaagccctgagctcagctgtttagctgcatgcgtttctaccatcaatggcatctagttctaagtgcttaatatatgctgtctcactgaataaatacataccttagggacaattattcaatttattactctcagtgaggttaactaatttgcctaaggctgcatatttgataagtggcagagctgagatttgaactcaggcctatatgacctcagagccccactcttagccattgtactgtcaaatgaccttggaaagacaacctaaaaggataatgatacaattttaggcctcaaagagtccccagaaaaggctttctctaatgcagagatttagggccacttaataggggtgtgtgtgtgtgtgtgtgtgtgtgtgtgtgtgtgtgtgtgtgtgtaaagacccctgaaatccaatttgaggtcaaccacctatgctgtctttacaccacatgagctagcctggacctgcccacctatttgctctgtgtctcaagccacttcccttcccatccccacaatcctcaccaccgactctggctcttggcaggtaggcttctggggctgcttggctctacatcatttgagtcactctgtccttatcaactttcatccccacagtatttttcctgtgaacagccatgcaaccaaaccacggcaggcaacgcgctgacatttctgaagagtcttctggaaattttccagaaagaaaagatgagagggatgagaggcaagatatgaagatgaaatattatttatcctatttattaaatttaaaaagctttctctttaagttgctacaatttaaaaatcaagtaagctactctaaatcagtatcagttgtgattatttgtttaacattgtatgtctttattttgaaataaatacatatgtggaaaaaacaacatgagctggtctcttggcaattattcatttcttgctgctcagaacaaagaaagctacaagtgttgttaaggggaagaatagatcagagactcctgtaggagtctctgtgataagactcctgatgctgaatacagaccctcaggctcataggctgtggctggagctgcag

SEQ ID NO: 54 - protein, 140 aa, Homo sapiens
MVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 55 - DNA, 4089 bp, Homo sapiens
atgcaccctccaaaatctatttgcataagcacacacacacacacacacacacacaccccagcagttcttgcctgcccagattcctctgcagctaaagtgatgaaacttactgggcggagcttcctaaaaagattattagggtctcctgggttggtgtgcctttaaacctttggactttaccacctcctatctctcctatctccttgcaacaaaggttaggagaacaagaatgcagaaaaaacgggtcctggatgacatctgagtgcctgctttgggcttcttgatgagtgagacagaaaataaaatacaaccccctcttttaaaagccatgcttactcaggttttccttcatttgcagctaaatacagaaatgagagaatattttggagcagggatggaagaagagaggtattccccttcccacaaccttctgatttcccagtacatcccccactggaaaaattcatttaaaatcagtataataagcattgattagatgcctactatgcatctgggcttgagggcaaactggactcaggccttttggcctcaagaagctcacagtgtgagagtggcatttgtgtcctcttgaaattcacaggactaaattgtgcccaggctgacattctatccatccataggtgcctgccttctcacttccctctcttcatgggctcttgccttgtaccaaaatccaaacccaaatctcctcacatgtgagtgttggcattcatgtctcagacatgacctatgggcttgggacttttccccgtggaccccagtgacttttcagatgaacaggtatcttcaaaaacttgagaaataggagtcctgtttgttgttcttgttgctttgtcaatataaggacacagggtctttattcaaatgttcatatctatctcttgacagaaatactatgagacatattgatggagaagccgttatctccatatgctaaatgaggacttgcaccagggaacttgcccatggttctctccaaccacttaaattctgaaattttgaaatgagagtggacagtaatttcaaatcaatggggaaagaatcaaatcttcagcaaatggcttgagataattagctacacatttcagaacaaataaagaagtcagatccgggccgggcacagtggctcatgcctgtaatctcagcactctgggaggccaaggcgggcggatcataaggtcaggagatcgagaccatcctggttaacacagtgaaaccccgtctctaataaaaatacaaaaaaaaataaaaaaacttagccgggcgtggtgccaggcgcctgtagtcccagctactcgggacgctgaggcaggagaatggcttgaactcgggaggcagagcttgcagtgagctgagatcatgccactgcactccagcctgggcaacagagcgagactctgtctcaaaaaaaaaaaagaagtcagatcctaacctcaaccctatttaacagattatagatgaaagaaaggtacaaatggcttttacatacctcccttctccctgacattttgtatgtgtgtgtgtgtgtatttacacacacatctcatataaggaaattgaagggaggctgcctgcatccctgagtcactctccctctccttctgaatgcttacctgtgcccagaccacctccttagcctcgcaccctccaggcttacagggcactcttctatgcccatcccaagtatagctgataccttccaagggccagacttggtgctaagtaccaagtacgcaaagattaataaaacaatgtcctgtttcagggagctcaaagctgattcggcagggcatggtgtgtacatgaatgataaccacgtagggttgcaggtttcctagtgaggtaagcacaaggcaagatgggaaacaaaggaaggaggggttcacagcctcacccagagtccagaacccctggcctgcctggtgcccatgctgagtccacttctggaacacccagctcagagagggggttagacctgcaggctaacacagacacagcccagaaaacccaggagccgagggggaaggagaaaggtgcaagaaggggaaacccaggtcctggtccccttctctctgcttcctggcagcagaactcagacagaacccttaagccagtctaagtctggcaggaccagtaagttctgagttagctccatactagtttctagcaggctctttctcacttcctgattcttaggtttctacattgacactccctgaagagttgggaagagacaccacagtcccctgaccctgatccataggtcacacagcagggacatccacagggtgggcgtgggccctctcatccctccctcccactcacttcacgctggctgggccccaaggtgtttgcaccccttgcagtgagtgaccttctctagtgcagcaagctcagaacctgctgccactggagttgtcccattgctgatgcagaaaggtgaagaactagcagaacactggaaatgccctccatctgggtccatggctacttaagctcaatgctccctggcaggcaggaggacaggtgctattgccctgttgggacagatgaaaaacagacacagggaggatgagtgatttgccctgactatagagtggcagggccaaggcagagcccaggcctcctgcacctaggtcagtgttcctcccagttacagtctaaactggaatggcaggcaaagcccctgtggaaggggaaggtgaaggctcaatcaaaggatccccagagactttccagatatctgaagaagtcctgatgtcactgccccggtccttccccaggtagagcaacactcctcgccgcaacccaactggctccccttaccttctacacacacacacacacacacacacacacacacacacacacacaaatccaagacaacactactaaggcttctttgggaaggggaagtagggataggtaagaggaaagtaagggacctcctatccagcctccatggaatcctgacttcttttccttgttatttcaacttcttccaccccatcttttaaactttagactccagccacagaagcttacaactaaaagaaactctaaggccaatttaatccaaggtttcattctatgtgctggagatggtgtacagtagggtgaggaaaccaaattctcagttggcactggtgtacccttgtacaggtgatgtaacatctctgtgcctcagtttgctcactataaaatagagacggtaggggtcatggtgagcactacctgactagcatataagaagctttcagcaagtgcagactactcttacccacttcccccaagcacagttggggtgggggacagctgaagaggtggaaacatgtgcctgagaatcctaatgaaatcggggtaaaggagcctggaacacatcctgtgaccccgcctgtcctgtaggaagccagtctctggaaagtaaaatggaagggctgcttgggaactttgaggatatttagcccaccccctcatttttacttggggaaactaaggcccagagacctaaggtgactgcctaagttagcaaggagaagtcttgggtattcatcccaggttggggggacccaattatttctcaatcccattgtattctggaatgggcaatttgtccacgtcactgtgacctaggaacacgcgaatgagaacccacagctgagggcctctgcgcacagaacagctgttctccccaggaaatcaactttttttaattgagaagctaaaaaattattctaagagaggtagcccatcctaaaaatagctgtaatgcagaagttcatgttcaaccaatcatttttgcttacgatgcaaaaattgaaaactaagtttattagagaggttagagaaggaggagctctaagcagaaaaaatcctgtgccgggaaaccttgattgtggctttttaatgaatgaagaggcctccctgagcttacaatataaaagggggacagagaggtgaaggtctacacatcaggggcttgctcttgcaaaaccaaaccacaagacagacttgcaaaagaaggcatgcacagctcagcactgctctgttgcctggtcctcctgactg

SEQ ID NO: 56 - protein, 14 aa, Homo sapiens
MHSSALLCCLVLLT

SEQ ID NO: 57 - DNA, 762 bp, Homo sapiens
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 58 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 59 - DNA, 471 bp, Homo sapiens
tactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagac

SEQ ID NO: 60 - DNA, 1831 bp, Homo sapiens
gctggagtgcaatggtgaaattatagcagactgcagtcttcaactcctgacctcaagcaattgtcctgcctcctcaacttcctgactacaggtgtgcatgaggactacaggcaggcatgtgccaacacatgcagctttttttttttttttttttcagagatgtggtctcgctttgttgcctacactggtctcaaactcttggcctcaagggatcctcccacctcggcttcccaaagtgcagagattacagtctcattttctctctctctgcattaatcaagaatgagagaaccctccaggggacaagatgaaggggaaatagatgatgtgcaaagaaatccttgctttatgaggggaaaaagtgttcctcatgaagttcaacaaaatgatgcaggtaaagcagttagctagcacctggcacatggcagacactcatagctgcctaaggcattggagaactggatcgtgctgcagccagaggcacctgcagagcctcatgggctggctgctgcagggtgtggctgattgagagtgcttttgtgagttggcctgcagggtacacttggtaacgtgccacagctctcaggaaagtgacctaagttggatttttctgcatggacatagaattgcaaaaaattctcatttgcatggagatggggagtttatttttcctagaagctgcatgtcaagacccagaagaaagaggcatttcataataatgattaatcagctatatcttaaagaagaaagaaaacaattaaggaaatacaatactaagaaaacaaggggaaaaaacaatctccccaaggtggatccacccagcaaaccttgacagcatttcctcttatccacctgaataaaaatgaccagccctttccaaatggcagagagcactgagaggagacacaaggagcagcccgcaagcaccaagtgagaggcatgaagttacagtgtgtttccctttggctcctgggtacaatactgatattgtgctcagtagacaaccacggtctcaggagatgtctgatttccacagacatgcaccatatagaagagagtttccaagaaatcaaaagagccatccaagctaaggacaccttcccaaatgtcactatcctgtccacattggagactctgcagatcattaagcccttagatgtgtgctgcgtgaccaagaacctcctggcgttctacgtggacagggtgttcaaggatcatcaggagccaaaccccaaaatcttgagaaaaatcagcagcattgccaactctttcctctacatgcagaaaactctgcggcaatgtcaggaacagaggcagtgtcactgcaggcaggaagccaccaatgccaccagagtcatccatgacaactatgatcagctggaggtccacgctgctgccattaaatccctgggagagctcgacgtctttctagcctggattaataagaatcatgaagtaatgttctcagcttgatgacaaggaacctgtatagtgatccagggatgaacaccccctgtgcggtttactgtgggagacagcccaccttgaaggggaaggagatggggaaggccccttgcagctgaaagtcccactggctggcctcaggctgtcttattccgcttgaaaatagccaaaaagtctactgtggtatttgtaataaactctatctgctgaaagggcctgcaggccatcctgggagtaaagggctgccttcccatctaatttattgtaaagtcatatagtccatgtctgtgatgtgagccaagtgatatcctgtagtacacattgtactgagtggtttttctgaataaattccatattttacctatga

SEQ ID NO: 61 - protein, 177 aa, Homo sapiens
MKLQCVSLWLLGTILILCSVDNHGLRRCLISTDMHHIEESFQEIKRAIQAKDTFPNVTILSTLETLQIIKPLDVCCVTKNLLAFYVDRVFKDHQEPNPKILRKISSIANSFLYMQKTLRQCQEQRQCHCRQEATNATRVIHDNYDQLEVHAAAIKSLGELDVFLAWINKNHEVMFSA

SEQ ID NO: 62 - DNA, 711 bp, Homo sapiens
tggggttccaggcgggcagcagctgcaggctgaccttgcagcttggcggaatggactggcctcacaacctgctgtttcttcttaccatttccatcttcctggggctgggccagcccaggagccccaaaagcaagaggaaggggcaagggcggcctgggcccctggcccctggccctcaccaggtgccactggacctggtgtcacggatgaaaccgtatgcccgcatggaggagtatgagaggaacatcgaggagatggtggcccagctgaggaacagctcagagctggcccagagaaagtgtgaggtcaacttgcagctgtggatgtccaacaagaggagcctgtctccctggggctacagcatcaaccacgaccccagccgtatccccgtggacctgccggaggcacggtgcctgtgtctgggctgtgtgaaccccttcaccatgcaggaggaccgcagcatggtgagcgtgccggtgttcagccaggttcctgtgcgccgccgcctctgcccgccaccgccccgcacagggccttgccgccagcgcgcagtcatggagaccatcgctgtgggctgcacctgcatcttctgaattacctggcccagaagccaggccagcagcccgagaccatcctccttgcacctttgtgccaagaaaggcctatgaaaagtaaacactgacttttgaaagcaaaaaaaaaaaaaaaaaa

SEQ ID NO: 63 - protein, 180 aa, Homo sapiens
MDWPHNLLFLLTISIFLGLGQPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 64 - DNA, 1049 bp, Mus musculus
aaaacaacaggaagcagcttacaaactcggtgaacaactgagggaaccaaaccagagacgcgctgaacagagagaatcaggctcaaagcaagtggaagtgggcagagattccaccaggactggtgcaaggcgcagagccagccagatttgagaagaaggcaaaaagatgctggggagcagagctgtaatgctgctgttgctgctgccctggacagctcagggcagagctgtgcctgggggcagcagccctgcctggactcagtgccagcagctttcacagaagctctgcacactggcctggagtgcacatccactagtgggacacatggatctaagagaagagggagatgaagagactacaaatgatgttccccatatccagtgtggagatggctgtgacccccaaggactcagggacaacagtcagttctgcttgcaaaggatccaccagggtctgattttttatgagaagctgctaggatcggatattttcacaggggagccttctctgctccctgatagccctgtgggccagcttcatgcctccctactgggcctcagccaactcctgcagcctgagggtcaccactgggagactcagcagattccaagcctcagtcccagccagccatggcagcgtctccttctccgcttcaaaatccttcgcagcctccaggcctttgtggctgtagccgcccgggtctttgcccatggagcagcaaccctgagtccctaaaggcagcagctcaaggatggcactcagatctccatggcccagcaaggccaagataaatctaccaccccaggcacctgtgagccaacaggttaattagtccattaattttagtgggacctgcatatgttgaaaattaccaatactgactgacatgtgatgctgacctatgataaggttgagtatttattagatgggaagggaaatttggggattatttatcctcctggggacagtttggggaggattatttattgtatttatattgaattatgtacttttttcaataaagtcttatttttgtggctaaaaaaaa

SEQ ID NO: 65 - protein, 189 aa, Mus musculus
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 66 - DNA, 732 bp, Homo sapiens
atgggccagacggcaggcgaccttggctggcgtctcagcctgttgctgcttcccttgctcctggttcaagctggtgtctggggattcccaaggcccccagggaggccccagctgagcctgcaggagctgcggagggagttcacagtcagcctgcatctcgccaggaagctgctctccgaggttcggggccaggcccaccgctttgcggaatctcacctgccaggagtgaacctgtacctcctgcccctgggagagcagctccctgatgtttccctgaccttccaggcctggcgccgcctctctgacccggagcgtctctgcttcatctccaccacgcttcagcccttccatgccccgctgggagggctggggacccagggccgctggaccaacatggagaggatgcagctgtgggccatgaggctggacctccgcgatctgcagcggcacctccgcttccaggtgctggctgcaggattcaacctcccggaggaggaggaggaggaagaggaggaggaggaggaggagaggaaggggctgctcccaggggcactgggcagcgccttacagggcccggcccaggtgtcctggccccagctcctctccacctaccgcctgctgcactccttggagctcgtcttatctcgggccgtgcgggagttgctgctgctgtccaaggctgggcactcagtctggcccttggggttcccaacattgagcccccagccctga

SEQ ID NO: 67 - protein, 243 aa, Homo sapiens [sequence omitted]

SEQ ID NO: 68 - DNA, 736 bp, Homo sapiens
ttcaaggttacccatctcaagtagcctagcaatattggcaacatcccaatggccctgtccttttctttactgatggccgtgctggtgctcagctacaaatccatctgttctctgggctgtgatctgcctcagacccacagcctgggtaataggagggccttgatactcctggcacaaatgggaagaatctctcctttctcctgcctgaaggacagacatgactttggattcccccaggaggagtttgatggcaaccagttccagaaggctcaagccatctctgtcctccatgagatgatccagcagaccttcaatctcttcagcacaaaggactcatctgctacttgggaacagagcctcctagaaaaattttccactgaacttaaccagcagctgaatgacctggaagcctgcgtgatacaggaggttggggtggaagagactcccctgatgaatgtggactccatcctggctgtgaagaaatacttccaaagaatcactctttatctgacagagaagaaatacagcccttgtgcctgggaggttgtcagagcagaaatcatgagatccttctctttatcaaaaatttttcaagaaagattaaggaggaaggaatgaaacctgtttcaacatggaaatgatctgtattgactaatacaccagtccacacttctatgacttctgccatttcaaagactcatttctcctataaccaccgcatgagttgactcaaaac

SEQ ID NO: 69 - protein, 181 aa, Homo sapiens
MAVLVLSYKSICSLGCDLPQTHSLGNRRALILLAQMGRISPFSCLKDRHDFGFPQEEFDGNQFQKAQAISVLHEMIQQTFNLFSTKDSSATWEQSLLEKFSTELNQQLNDLEACVIQEVGVEETPLMNVDSILAVKKYFQRITLYLTEKKYSPCAWEVVRAEIMRSFSLSKIFQERLRRKE

SEQ ID NO: 70 - DNA, 840 bp, Homo sapiens
acattctaactgcaacctttcgaagcctttgctctggcacaacaggtagtaggcgacactgttcgtgttgtcaacatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgaagatctcctagcctgtgcctctgggactggacaattgcttcaagcattcttcaaccagcagatgctgtttaagtgactgatggctaatgtactgcatatgaaaggacactagaagattttgaaatttttattaaattatgagttatttttatttatttaaattttattttggaaaataaattatttttggtgcaaaagtca

SEQ ID NO: 71 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 72 - DNA, 750 bp, Homo sapiens
atggccatgctcagggtccagcccgaggcccaagccaaggtggatgtgtttcgtgaagacctctgtaccaagacagagaacctgctcgggagctatttccccaagaagatttctgagctggatgcatttttaaaggagccagctctcaatgaagccaacttgagcaatctgaaggccccattggacatcccagtgcctgatccagtcaaggagaaagagaaagaggagcggaagaaacagcaggagaaggaagacaaggatgaaaagaagaagggggaggatgaagacaaaggtcctccctgtggcccagtgaactgcaatgaaaagatcgtggtccttctgcagcgcttgaagcctgagatcaaggatgtcattgagcagctcaacctggtcaccacctggttgcagctgcagatacctcggattgaggatggtaacaattttggagtggctgtccaggagaaggtgtttgagctgatgaccagcctccacaccaagctagaaggcttccacactcaaatctctaagtatttctctgagcgtggtgatgcagtgactaaagcagccaagcagccccatgtgggtgattatcggcagctggtgcacgagctggatgaggcagagtaccgggacatccggctgatggtcatggagatccgcaatgcttatgctgtgttatatgacatcatcctgaagaacttcgagaagctcaagaagcccaggggagaaacaaagggaatgatctattga

SEQ ID NO: 73 - protein, 249 aa, Homo sapiens
MAMLRVQPEAQAKVDVFREDLCTKTENLLGSYFPKKISELDAFLKEPALNEANLSNLKAPLDIPVPDPVKEKEKEERKKQQEKEDKDEKKKGEDEDKGPPCGPVNCNEKIVVLLQRLKPEIKDVIEQLNLVTTWLQLQIPRIEDGNNFGVAVQEKVFELMTSLHTKLEGFHTQISKYFSERGDAVTKAAKQPHVGDYRQLVHELDEAEYRDIRLMVMEIRNAYAVLYDIILKNFEKLKKPRGETKGMIY

SEQ ID NO: 74 - DNA, 1669 bp, Homo sapiens
ctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaa

SEQ ID NO: 75 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 76 - DNA, 37797 bp, Homo sapiens
cctccttagacaagcttctacatgctactcatctctccatcaaaccaccatattcgggctttggccatctgctctccacagccaagtccccagtggcctctctgcttctgacacagtgaaagccattcagatctgtcttgttggcagcattcctcactttgagcagcgccctcctactaggatacccctccttgactacaaccccacattctctacttcctgggctcttctgtcactggaggatgactcccaggtgtgaatcttcatcccgcgtccctcactcaagcccccgatcctcatatccagctttatcctcatgggatgcttcaccaggatgagtcataagcacctcagactcagggtgtcccaaaccactcatctacctggcaagcctgcactctgcatgtgcctcattctgaacatggcaccatcacctgctgcaatgtccagaccacaaacaccctacaatatccttgactctcctttctccccttctccctgtatacagactccaaattctattgagactattacctcctacacccctcacatttgcccagccttccccatctctgcctctaccaccatagttcaagctctcccatggtcccttcctggttacctgttcttcttgcctccttaagcctctcatgacactggccatgtcacttgcctccacccatcacccgctaggctcttagctggagtctgggccctgctaccttcctccccttcttccctacccttgactccacctccctgtgcttcagccaaccagataacttgagtttcgtgaatgcatgcctcagtttacctgattaactcattttcatctttcaggcctcagagcaggtatcaccctgtcagggccaggtgcctcttcttagctcccaaagccccagctactcttcatggaacatcattggcttgggctacggatcttcccaaattggagctttttcacaaagggcttaggtctcactcattctattaatccatctgtgtctccccagggctagcagtgccaagtaactgacgggtgattaatagatgcttgggtaagtatcacctctttaccatgtgacaatttgtttacctgccttgagctcctccagggcaggactcttgcctttgcagaatctatctggcaggtactgttgcagagatgtttactgaagaagggaatgaattagtaccaaggtgaggaccccacccttccccacgggctccaaaagcagcttagagcccaacaaaacctgccccacatttttggcgtttctgtggatcacacgatttactcatctgtctttcaatgagcatgacaggtggggtgggggtggagggattagagattgaggagctggggagggtggtcagctcctggggtgcagaaacaagtctgatgggccatggtgttctgggaatcagcactgcctcccctcacccctccctgcagtgttttgtagcctcaagatcagtgagggaatcttcgggcccccagcatgcaggaccgaagcccccgagacagctgtccctcagtcccaaggtccccatttggaagcagccacaggaggcctaagggacctatacccttggtttgaggaagactgtggcgagggagagagggagggagggctggcagtgagggcaagggctgggaaaactgagcacgggcacagtgcgggagcgggtgggtgcccagggcagccaggggcgcacgggttgggaggcgccaggcggcccgccctccttgcacgggccggcccagcttccccgcccctggcgtccgctccctcccgctcgcagcttacttaacctggcccgggcggcggaggcgctctcacttccctggagccgcccgcttgcccgtcggtcgctagctcgctcggtgcgcgtcgtcccgctccatggcgctcttcgtgcggctgctggctctcgccctggctctggccctgggccccgccgcgaccctggcgggtcccgccaagtcgccctaccagctggtgctgcagcacagcaggctccggggccgccagcacgggtaagccgagccgcctggccaggggctgcggaaggtcaggtagtcggggctcggagcgcaagccgctgggggcattgaactgggctgggggcgcaggggacaaagcccgaactaaaaaccttgcagcgtggagcgctcggacaccagccctgcacgcggtggaaggagagagggagggaggtggaggaccatggagggaaagcgggaggccgccgctttgtagaagggagtggggaagtggaccagagaccttcgactcaggccaagagcctgagacggacagcgctttcagcttctcctcccagccactgcagaaagggggaaatggcaactctttggccataatcaccgggggagggtgccaagggcaaagcccacccagcagtacacctattccaacccagccaggcccccggccagcgactccagacaagaacctgggccacacacggtggcagcatctaaggtgccccaggctcctgtgctcctggccaggccctgcactcagacactgctggcacccgacactgctctctgggtacagcaagggcaatgtggcacttcttgtcctgcccgatgaagagcaggagaatgcactgggccctcacacacactgttcaaatggggaaactgagtcctgagtggttccactttcccacagtcctgaagtgtgcactggagccaggattggagtctgtcttaaagtaatagctgggtttgtaaatgtaggacactatcattgcaggaattcctttgagaccctgaagatgtgttggctttaggagacaaactcaagcagaaggtctggtctgatagtggccctaatactgacccaggcagaggcaggcaacatttctacctcaaaaaccaggccatacctgcgtcacaaatacccaggctttgctgcagcttccagcctacctggttgcaccaacttctttttcataactaggtaaaactatatatgagtagaatcttgtagtgactcctcagaggaagcctaaataccatcggggtctggcgttcacacccacaagcaatgcccaaacctccaagagactgggcagatctgtgctcaaatcaaaactcattgttgggggtgatagagttgacttcacaggccctgaaagtcttggctccttgcactaggagtgctctgggtacgggtacaggctgccccttgtagggcatagttgctcttgtttcctctacttgtggctttatggtctaggcctttcaggagtttggggctctggcggagagggcctgctgggagcacatctggccaccctgcagagtgaaatcaaaccaggcctggctgcaacctcaacaccctcctggaaagaggagaatactggggatatcctggggtctttctggaagtgggagaatcagctttgacttgggcagtgtgcagaatagagtgaggggggatgtcagaaagatgagagggatatgaggcctcaacatcaaaatgcaagcacctggcatttttattatctctgcccacctctccgttggtctctctgcctttcctgccaatgaattgtgttatgtttgggtgcctcaatttgcctaggagggttctatttcttctgtatcttcgccactaagtcaggagaagatccttatagcatgccctgcaacagtgtcacctgtaagggcatctctctgcacagccacagtgaaggatcctcaaaggtattgagggctttccatcaagagccatctttacagcaaacctctttcccttcagagcccagaagagtgctgaccagctggaaaacagggtttttttcttaaatgcagatgctcttgattatgagttccagatattagatcaacttccccaccatacccctgcaggcaaagcctcttaattagcttcctgcagcacagctggaaaggcctattgtaatctgtgatgggcagagtaatctaagaagtcacaggagcacccctgtcccagtagaatctggatgcgcaggcacatgaaccatggcaaaatggttgcaggcacagttgtatttactctgatctaactgtccctgttaatgccacagggctgcctggcctggcacacagggctgtggcgccttgtgcaaatggataacgttgttctagctccagcctttcattcaaagtgaaaactgttagaaagggaaggaaaactttgctattttaaggaattgtagcgtgctgcctgatatgaaggaagaaataacagctgtgccttgcttgtgcgcagcactcgattgccgcttttgctttcgacctcaccacaacacagtgagatctactgttcatgttcccattttacaggaggtgaaactgcagcttagtgaggtagagagtgacttagttcagacacagaatgctgttgggagagtaataactatgatatggtctcttgactcccagctatatctgtgttgctatagggaaggggaaaaataatactgaaagagaagtaaaaatacaatcacacttccaaacatcaaccaccaaaaactgaactgaatttcctgaagcacttggttttcaaatctaagctgaacatcaatgctgttattcttgaggcccagaagcaacttgctcatttcaattaagcttcagcatgaacttcctatgtacacagcccacccacactccccgatgtgagaaggagagggtcacagccgcccccagcctctgctgctgccacaaggacagcagcagtggaaacattcagcaaaggaatgttggagccacatccacaagagactcactgaagattcgccaaacgcctacggaaagtggcagggaattcattgacagtaattgtttcctgcttgatcagattgaagagcttctgggattctgtaacaataaataggaccgggggctggagtatggccagcaaggactcttcaggggttattcagggactgtctaacctgtgaatcctaggcagcaaacagaaaccaggtattcagaaatctggaggatttggtcaggcccagctaggactagggaggcatgggcctctgctggctgtggtcccttctccagccttcacttctcttgtccctagatccttacatggattcattaatgctcattgtccctcctgggcccactcactttcacctgttgaacaaaaaactggccaagaggtgacagtcatatcaccgcagaagagacagggcagagaaatgaaggggcagaatggactcccacccaaaagcctgactctgaatatttgagaattgttcaagttcctgcagaggaatcatgatggggacagtaggtgtagtttttactgcaatattggtgtcttcttaacaaatacgctgcacatcaagtgatgtctgtggatggcattcttaaagtaacagggaaattgatgttaaagaaatacttcatcctttgggtgatacctgaagttctctgagcttggaggtcttgtgaaagccctcagtattgtttgttttatttgctttcctctgacttgtgattcagtcagatgcatgcctgcctctggctcaggaagatcaaccctctcctgactgaccacgcctctcctgactgaccacgtagcacagcagcttcctttccctaggggctcctaatgaagctttcacaatcacctggcctgagcacagtttgggtcaggacttggtatatacttgaaaaaaacatgcaaaaccaaaatcctgtggttctggaaaaggcttcttagcagaacccccagacatttacactctgctttttcacagggtccctgaggattctttggatctgggtagtttggggagcagtattttcaacaagttcatttcgtgctccttctacaccctgcctggatgctaggccccatctagaatgtgaacaacagaacaaggcagaacacttgtcctcaaggttctgttgagtgttagatgcagagaagagacaccccccacctccccgcatcacttacaggaattctgtttggaacccaacatcaaataaggaccgtatccactgtcagaggatgggaagcagcatgtcatctgggacattggagaaaggctcctgggggaagtgggacttgagctgtgatctaagtaatgaacaactgagagttaaatgggagagcatcccctatcagggtcctgagagcaaccagccatggtttaaaccagctataaagcctcgggtttataggatagacagtaacaatggcttgtctttgggagccaagcagctggtccaggcatgcagagcatgtctgtatggagagctgcctgagagatgcttttgtttacacttatcaattgcccatgtcaaagaaggatatgtacatgaagttacatcagtatgtaagagagattttaacaatttttgcaggggaagctttcatgggggctgatgggaatctaggtaaacagaaccaaagtctaaacccaagatatccccagtaccaagactgaaatgactctctcctctatctctagaaagttccagtgacccaaggaggcaaacacgatgggagtcattaaagtggggtggacgtgctgatcatcttcctaattctgctgcttttgttttcagccccaacgtgtgtgctgtgcagaaggttattggcactaataggaagtacttcaccaactgcaagcagtggtaccaaaggaaaatctgtggcaaatcaacgtgagtatctgtaaccagccaggagaccaagctgtatgcacgctggctgcagttccccagggcctgggccagccttctagaaggtcaggttgcctaaaaagccatgaagatgcatgtgcgaacatgtctgggacctgcgtgctagggagtggcatttttaggaagctggccaattttgttttgcatttttaaggctgctgacaagacttggagacatttttcagggctggtttgggtttgcaagaaacatgaaacactgcgtgtgtgtgtgtgtgtgtgtgtttctcaatcctcataaaataatacagatatgcagtggagaagccaccagcatgtgactctggaaaagaaagcccattggtgaatctgtactaaagaatgccatccctatcttacagtcctaaggtaaacaccccaaaaagacttagagcactaaacatatgcagattatgagacagcatagcatataatatttgcacagacttcctcattcaaaccctagctctacctgggccagtcgattcatctttagaaccctccattgctttacctgaaaagttcgtataacaaaaggacccaccttatggggttgttacaaggattgaatgaaataatgtacataagagactgaatatggtgcccagcacatatcagtgctcaataaatgctagctactattattattatcaccctagatttgcaaatctagaccacacaagcagaagtaagagtgccaacggggtgtggaccagtgtggttacaatagggcttgttgatgtctgtttcagcaaggagggaggcagcttttaccccactgcccagctccctggtggaatcaggtgcatgttctaacaattctggggaaacctaatctgttttggcactgtcaacagatctcaaagctggctgtctcctatagctaggaagatgtgtatgacaaatctcctgagccacttgtgaaggcctgaccttcctcctgtctccatacataatgggatgattaagaaactctaagccactctcttaagcacttttcaatgttagggatttttaagtttattgttgtgacattgcttttgagcagacatctcctccaatttaatagccaactgaaagaagagaaaatgctctttccttaaactgtatgtggaaataaatattccaatgtgtgaccctgattatgttaggcaattagcaatcctaatatgaattgagggaagttgggattcatggcacagctggggagataccagcagtccctgggagcctgtccagggcaggtccatggcagcttgctccatgcctgattgacagcccagcctgcaagctaaaagttgagtgagctaggaggacacactgccaagattcagctaacagacacccagcgatattcttgctgctatgaacaaaaggagactatgcaaattatacaccacccattcttccaggatgcctgacttaaaaaataagaaaaaagatgggccgggcacagtggctcacgcctgtaatcccaacactttgggaggccgaggtgggcggatcacaaggtcaggagacagagaccatcctggctaacatggtgaaaccccgtctctactaaaaaaatacaaaaatattagcgggcgtggtggcgggcacctgtagtcccagctactcgggaggctgaggcaggagaatggcgtgaacctgggaggcggagcttgcagtgagccaagatcgtgccactgcagtccagcctgggtgacagagtgagacaccgtctcaaaaaaaaaaaaaaaaaaagaaaagaaaacctttagtactgattgattttttcccatgtgtgtatattatctactcaaattaacaattaattacttaattaaacacaaagccaggcctcacctaattgcttcttggaaggtgaccagagtgctagtgccaagcaaacaactcttctatatctcaagagccctgggcttcagagggccatcttttttgttaattcaagtttctctgaaaatggagacccgtttatgatgacaagctggctacagggtagcatctgccacactgtttcgggggtgccgctgggctgaagcatttgcccagctagttaacaatagctcgataacattccctatcagtgtccaggctgagaatactgtcagtgatgagtcgccttggctcttgtacctgtatctttgtgtgccaggacaaggcacaagcaacagagctgtgtgttgccaaaatgttcctgatgagcaggtcaacccctcgggggcaggtttggatatgataatgtggtgatgtggtggcgcagctcccttacccagtgagcacaaggggagtcctctaggaaaaggaagaaatgtctggatgaggtggggagatggggttcagagtggactcaggcaaagcccgatgcccagtcccagctgttggcctagtctcacaaagccagaaggatatgacatttacattcaactcttgaatttgtggccactgctttgggcaacttcaaagagagaaaatgaagatagaaaaatattatttgatataaaacttctaggacaagagaggcccttcctggaacattacatgtagtattaggaaggtggagctgccctggaaaagatccagagaactcagagagaggaagaggtggaacccatctctgttcttgtagagagctcagtaagagtggcttggcagggctcctgtgtacctgagaccaagaccagtgaggaggctactgtctgaccaccatacggtcagaattcagtgccatgggtggtcaggtgggaaggggagaggactgtgctggctggagttgatgttatcctggggaaagtaggtccctagatgcctttagttgagtgaggagcagactgggaaatgggagcacagtagtggttggggcaaaaaggactgtctctgcatgaggtccataggcagttggaattttctcagcaagactccagagaaggaggctggagcagaggtgtatgttgggatgaaaaggagtaaagtatcatgggggaggaggcagctcaggttgtcaagggtcaagaaaccagaaggagaatttcaccttggaagcagacaacgggtaccaagcatacaggggaatactttgtggtgagaggtcacacagagatacaggagccgacctggtgagacaggagcctggagccacctgcctgcttttgtgaggccccagactccactgctatcatcaggtgaagctctgttgcctgcacacaaaagcttttctgcatttacaaagagagaagggcctgagtttctggtgcaatgcgtcaagctgacatatggactttattacaggaagtggttaccagtgggtccctatttagtggctgttattgtgaattttattgttcggaaattcactttagcatttatttcagatcctaaatagcaccggagtgatacaatggctaatcaaacaaagagggctgtggggagcagacagtcagcatccccctctgtgatttcaggccctggtttgattagtagccataaaattttttacgtgtggcactttgagcaaaggtgcaggaaattgtggtcaggaagcctggctgcctctcgacaggcttcctttgtgctagccccagggagaggaggcctatttaacagccaagtccaagttgacatcatgggactggaatagtcatagcaggagctcagacatcataaacgtggcatagggagggctggtggaggagctagcgggtatgggtggcagctattcattccaaaagtcttgaaattgtttcacgagcaacacatttcacaagtgcgaagcccttctctggagccaagatgagctggcagagcactcctgtttctctagtagcaagtgttcctttgcccaggggcaaaaatattaatactccttcagcactgcattaatgcttaaagatttaacttttaaagagatcagctggtgcatggtcgagcttttccatcagctggcagggctttttcagtaggtgtccttctgggcagggcactggggacagctgacgtgaaggtgaagaagagctgtcgttttcctcccttatatcccacaaccttggtcccaagaggaaaaaaaagaagatggtgagaagtcatccaagcagaccccagacccatactagtgcctcctttcctgtttcatatccctgtgcagccagctgggatctcttgaataatctgctctgggggcactgagattggacatacaccaaacagcggagatcgaccaaacgcctctgttgggcagtgtttcctgagggttctgtcccattctgtaaactaggaggctgactagctgacaaggaattttattctgttgggtatttacatgaacctatgtgccacctggggtaagaccctgtggtaggtagaaacatgacttcccaaaaatgtccacatcctaatctctaattctgtaaatatattcccttactggaaaaagagactttgcaggtgtgattaaattaaggatcataagagggagagattatccaggattatttgatgagtctaatataatcatcagggtacttaaaagagggaggcaggctgtgcctggtggttcacgcctttaatcccagcactttgggagactgaggcgagcgggtcacgaggacaggagttcgagaccagcctgaccaacatggtgaaactccccctctagtaaaaaaaaaaatacaaaaattagccaggcatggtggtacacacctgtaatcccagctactcaggaggctgaggcgggagaattgcttgaacccaggaggcagaggttgtggtgagctgagatcgcaccactgccctccagcctgggcaacagagcaagactccatctcaaaaaaaaaaaaagagggaggcagtgggatcagagtcagagaaggcaacgtgatgatgaaagctgacatttgagtgatgcaaccacaagccaaggaatgcaggcagcttctcaaagctggaaaggacgagcaatggattcttccctacagcctctgtgaggaatgcagcctttgattttaaccccataaggccgatttctgactctagcctctggaattgtaagataatttgcatgatctcaagccactaaatttgtggtaatttgtcacagaaagcaatgggaagccaacacaggccttatttgttgacttatagatgcatttttctttatttcaatgtacttttatcaatggtctcatgtagggtattgctttcaatgaagatattaacatagtttcaactttaaggtttatatctggagtttctttagaagcttcacaactgaccacttagtaaacagtaagcatctgttaagtgcttctcatatgtaagttcattcaattctcacaatcacactataagataaatatgattattagcccatttacagatgaggagacaggctcaaaagacttttatgcaacctggtcaaagtcattcactggtaagctgaggaggtctgtccacttccttttgctgcccccagggggtatcaagcctggcagttagtgtcagcgacttaggaggtgaacaagtgagcaggcctgtaggacctggctaaactgccccaggtctctgtctacagcctcaaacctgtggctgtgggtcccagagacaaggcctcctcagcatcagagaaggatgcctttgtctcagggtcatcaaccttctccaggttgctcaccccctgctgtaaaggggatccccaagaccgctcatcagacaaggagcttgggaactgaggagacacagtcagcctccaggagtgcccaaaatgccctcacatgctgcatacagattgccacaaataaagtacatccacattctgaagactctgtcctcatcaccaaccaggctggcccctggtgagggctgtagtggttgaggcctttgttggtagacggtaggttaaagcaagccatgattttctattgggaggcttcagaatcagctcagctgtgtttccaagaccaggagggcagaaagcaaaccatcccaggcaagcagtccatgggccatgtcagatgtctagacgttatgggtctgtgtttgctctgccattcctctcggaaactatgatgccctgtatggtttaccttcagtcacaggtgactggcctacagggccattccttgttccaacgacttctcgagtataattaatccccaggcatttacggccagagcagccggccaaatccgtgaagtgcagtggttgttttaaattatattaacttcttggaaacttattttagggagagaaaactcagtacttctctctatccaatcttgagtaaaaatgttagaagggactggtggagagcctcccagacatccctacacatagactttgggttgacattatctctttgcaccttccttgaaactttcttctaaattaggtgccttccctaatttaggcaccttcccagtactagtctgtgacctgttaggaaccaggccacacagcaggagttgagtggcagggagtgagcattattgcctgagctccgcctcctgtcagatcagcagtggcattagattctcatagcagtccgaatactattgtgaactgtgcgtgtaagggatctagcttgtgcattccttatgagaatctaatgcccgatggtctgagatggaagagtttcataccaaaaccaccccttccccctgccaccatctggggaaatattgtctaccacgaaactgatccctggtgccaaaaaggttggggaccgctgtcctaagggatctgctttttctgacctgaggtttttctttattagactgtatctggctgaggagaagcctgaagcctttaatcggaacagctttggctgatgagattagattcagaaaccaacagattggtcttttctatgcagggaagcctaggaactggggggctatggctgggaagccccctattgtttccatcctttcctatgttcatcctggaggaatggcatcagacccatgcctctgtgattgctcccagcccatccaaccacagcatctatgttctgcctgggaccagggccagggagcatggcacactgagctgagtataaggagagtggagcaggccactgccagcccagaaaattttggtcaaagttgcctgaaatcttctcagccttcgattcacagctgctctctgctgctctggggccatgcagaccagttcagaaaagagttaatttgttggggcagttggaggcaggtggactgccagctttgacaccttcccagcccacaggctgctgcactggggctgaaggcgtggctaacccctgcacacctagagagtgacagagatgccagactgggcagcaggaaggcaagaggattaagagagagcttcctggctgaaagccacactcggttaaccaggaaaaagcccttggcacgagaagactcagtggcctgagggactgagccttggttgttgggcatgtgctgcataagccatccatgtgtgacagtagagtgtagtccagccactgtgggacatgggtgctgaaagaccacatggagaggaacagtgagtgctgacaagggctagccttgatcactttggagacaccccctgtgtcttctagatgtcagactttccaaatctgtctgctatcctccaaacgtgcattttcaagagcaatggaaaaaggattggacttgatggaatgcagcaagagtcctaggtctgttactacctacctatgaccttaagaaactccttcacccctcagaacccttacagctttctttctgattctatcatgagttactctactccaagctgagacttttctgcttagatctatcccttcctcctaaacccccaacctccatttctcctggtgtctttctttacacacccctcagcatacacacacacctagccacaggaaccaatgagttaatatttgaggagttggttttcttttgtcctcaatgagatcctggtgaggccacttgagctgttcagctcccttgcggtattttggggatggaactcagaagccaacaatatagaaaaagagtctttggccagctttcccaggggctccatgccatagagagtactgcacccgtgtgcacagggggccctgacatgaggactttgaggataacactattcctccaactctgcttcagcatctccatggattttcacacagacactttaggaaagaaactaagtttggggggacttgacctaatcccacatcccagccccagtaatacagccctggaatttatcacagaaagcctagaatcccatgcatatcccatgcatatgcatccctagtcctatgggttcaaggcttggagctctccctggatttagctgggaaaagttggcagacagttcttctctgtcttctagaaatatggactagaatcgtgagtgtgagattgcaagtaacttttaaaatcatctagtttaacttcaccccatttcatagaccaagaaactgagaccagagagagaaatggactttcaagttcaccctgctagttactgatggatcacaagtcaaatctcctgattctagcactgtttctcttacaccacaccacctttgaaagtgtgtcaatcaaatcttactttagttgcagaggatgactttagtttctgaagataaaattgtgagtcaatcaagatcagtcccaagacaatagcctgtttagcccttataagttcagggatgaaaggttagaaagaaacaggatggaaggaggactggagaaaaaaacaaaagaggaaggaaggaggaggaagcaaacaggaaaaaaaaagaatgtgcatagcttgtcactcctcagtcatttcctgggagcccatttctagcaaagtgacagctgcaactccctggccacctgagcatcttagctgatctgtctctgaaacaccccctggagaacagatgaatcaggcttcatcttcgcttaactaagtcttccctgagacgactccatttaaatgaacaagagcaggatttcctgggcacactgagagcaccttccagaggcccctccagagccctaaagcctgtatttcttccagtcggcctgtttctttcctggtgatgtcattaaacgccctttgagagtcccacagtgagcagttctgcggtaaaacccgctgcaattaaagtctgagtcctttcctgtctcaaagggcatattcatatagaagaaaggaaaaggaaggactggctgtttgcatttggttccaggcctgttgagtagaggtcgtgctcactccaccgaaggtacagggtagccttcagcagaacctggggatttggttttaagcaagtctttcttaggtgtgggctttcagaacacttccttccttgcaatattatttgaaattctcagtgttttagccgtccccagaatattggttcgttaaagctgtgtatttcagatctccagacagtggtcactgtttgtatattttcaatttcaaaccagaaaacaaaagttcttattgattactttttttatttaaaaaataaaaagtaagtatcttcgtaagaggagctttgttttaattttaaagtttaaaatttgattgtgaagacagagaaaaacttgatgattgtagatatattcccctctttggctattcaatcagagaactagaaaatcatgagagatttaatgaccactgcctgatacacatatgtgttttacagatgaggaaactgagacccagagagatgatgaaattggctgaggatggcccagctggtcagtgacagactcagagccagagctggtgcagggctctttctattccttcctgttccctttcaggaacactcaccatcggctttcctgtgaataatgttgagataaaatccttggtgcattatgttttctagtcacaacattgactaggctgccagagtcctctgttctcccagttggttggctgtaggtgttggcagccgccaggagcattctacagaacagaggaggagtgagactctccttgctcaggaaaggcagacctatgacttagcaaatgactcctaagaggagagtgtttcacccaccattcctcttccttggctgtggaggcaacttagtggagaggggccagatgacctgtgaggaacagtgaagccctgcctaacacaatgtatggttgtcttgttacagagtcatcagctacgagtgctgtcctggatatgaaaaggtccctggggagaagggctgtccagcaggtgaatgaatcctccgggccttgcctgttggtgtgggtggaagggaatggtgggagagaggagtacccacataaaaggcagcagagtgtgaatgggggcagtggcacaaggacatggcattctccccacgtgcccactggccccaggctctatgcgaggggctgaggaatggaagctggaaacagcgcatttcctgagctgctcctcctggcctccttaccacactggtggagtagactccaactgtggcctgtccatgcccttcccagcaggcacaggctcaggctcaggctcttggcctctgcctctggctgggagtgattctaaacacatccagcagggtcagcctgatagcccatcagtttccgatcagctctgctagagagccgatgggatgtgggaggagggggtcactggtgggctggcaaccccaagccatccccatctccctctgtgtctaaacttggccctttggagttcggtagggagaagagccataggccaggtgggctcacccagagtcagcagagagtcccacaaatggttgcactgggcgaaagacagcatggcacctgtgaattttattagagcttttcttttagtgctacacacaagtgactgtacaggggagttagtattttgttttaattttgaaatagagtcatcttttggtatctgcgggggattgattctaggacccattctaggatgccatatcctcagatgttcaagtccctgatataaagtggtatagtatttgcatgtaatctatgcatattcttccatgtactttaaatcatctcaagattacttataataccaaatataatgtaaatcctatgtaagtagttgttataccctcttttaaatttttgtattatcttttattgtatttcaaaaaatatttttggtccatgtttagttgaatctgtgggtgaagaacccacagatacgaagggccaactgtattggctatttttttagttaagaatgtgagactgaggccaggcgcagtggctcatgcctttgattccagcactttgggaggccaagaggggacgatcacctgagccaagaattcgagaccagcagcccgtgcaacatagtgagaccttgtctcttaaagattgtgagactgggctgggcacggtggctcacgcctgtaatcctagcactttgggaggccaaggcaggtggatcaactgaggtcaggagtttgagatcagcctggctaacatagtgaaactctgtctctactaaaaatacaaaaaaattagctgggtgtggtggtgggcgcctataatcccagctactcaggaggctgaggcaggagaatcgcttgtatccaggaggcggaggttgcagtgagctgagatagggccgttgcactccagcctgggcaagaagagcaaaactccatctcaaaaataaataaataaataaataaataaatcatgagactgagacataacaggaaggagggcaatttggttggttccaaggttcctagagtatgtgatgggagaggttggtgcgggtggggccatggaggtactgactcaagtggagggacaggtggggaaatgggatgggaaaagaagattgaccttagaaggggagctcaacctctgaaccctaatttcagacccttcaaaatgaatattaagctcattttggtctaagaaacaaaaaacaaatgaacatgaaactcattttggtcttataaggtctgagaaaccccttctaaacttcaagctgctttaagaaataacattttattacctgcaaatacacacagtactttggagatttataatagtctcttattctaatagaagccattagggaaccagtttcaataaacaggtaaatctgtaagactagtttgtaattaggatatctgtttccagtgtccattcctgcctctgttatctaaatgtctgggaacaagagctgtgctctgctgtgtttaaaatgattaaaaatcaccaattagttgagttcacgtagacaggcatttgacttattgagttgttttaagaagactataacaagccttaagccccccagaaacagcctgtctttgggctttcccacatgcctcctcgtcctctccacctgtagatgtaccgtgctctctgtcagagaagggagggtgtggttgggctggacccccagaggccatccctccttctgtcttctgctcctgcagccctaccactctcaaacctttacgagaccctgggagtcgttggatccaccaccactcagctgtacacggaccgcacggagaagctgaggcctgagatggaggggcccggcagcttcaccatcttcgcccctagcaacgaggcctgggcctccttgccagctgtgagatgacctccgtctgcccgggggactcttatggggaactgccttacttccccgaggggtgggcatgatgaatgggagtctgcagtcatttcctactgtttcaggaagctttctccttaaccccttagaaaaggctgtggaacttgagctaaaatatgtcttaccaggttgcgtctaatgccccccgttccctactgggcagaaagacttgggtgcttcctgaggagggatccttggcagaagagaggcctgggctcacgagggctgagaacatgtttcccagagttgcaaggacccatctcttaaacacagagtctgcagcccctaactgacaccctgtccttcctcctaggaagtgctggactccctggtcagcaatgtcaacattgagctgctcaatgccctccgctaccatatggtgggcaggcgagtcctgactgatgagctgaaacacggcatgaccctcacctctatgtaccagaattccaacatccagatccaccactatcctaatggggtaggggatccccagccatactgcatggcccttggtgcataatgaacccatttctgttccatgtgtgggctggtttctggggtttaagctgtagacaacccaccctctttgtgcctgcttctccttgggccctctattccacagcttgtggaacccacattttgctactgtgtttgaaaacactgttttctcctcccggggctttgggactatgcctctgttgtgttgactgctcatccttgctgcttctctgggcagattgtaactgtgaactgtgcccggctgctgaaagccgaccaccatgcaaccaacggggtggtgcacctcatcgataaggtcatctccaccatcaccaacaacatccagcagatcattgagatcgaggacacctttgagacccttcgggtaagggactgccctgggtggaggcccaggcttgggacacattgcctcccaagaggggcctagcaggaactcttctgcaggagaggtagaggatggctcctgtaggggaacatagagcaggttcccctgaatgcccttgaacatggagaattcattgaccagacattcagcttgacctaacctgtgaaattctccatcttctttataaagtgttcccttccttgcctcccctggaaaggtcagtggtgtgtggctgcagcagcacagtgtcctctgagccctggacctgcactgtggcttccagaggtggcagttcccacatggggtactagaataaatggcctatcaggctgtgtgtgctttgggatcacatgtccccaccctaggaccctggttccaaccatacgcatgttctcttggagcccagaacagcagagaagccaccagtgtggacacagaagtcaagggtctgatttccagcctggcttctgactgctctggggccgcaggaatacggttccttcccccatgcccagcaggcatttgtcttacaactggaggggaaggcatgttcctcttggcaaggactgctcaggaggaagtggaggcaggctgccctgtcagggtttttgccttgattcaaggagaacttcctaaccacaaaggatacaagtgggagtgaggcggaccctccctagagatctccaacacagagagacaaacacgctggggctggctggcactgacaggcctcgcaggtgtggatggctgttagctgggagcttcgctgtctaagctcctctcccatgcttttcttctgggttgctcgaaggacgggggtctgcaagaaaatgatgttcccacatagttggcagcacgtgaacagcaattgatccctttgcatcacctcctcttactgtttagatttggtaaatatttcttccttccctcttctgaccctccattttgccgatctttccttcttataacacatacttactaggtacctgctacttcccgggtgggcctatgtgccaggagtatagaggtgaacaaggaaggcaaagttctattctcagtagagctaatactctatctggagagagacaacaaacaaatcaacaaggtagccaggggctgtgataatttatgtcaagtgggcaggtaaatcgggagtgacagtagtgcagggaggattggaaagtcagggagttctctctggaggaggtggcttttgatctgcagcctaaaggatgagaatgggtccattatacaaaatgctggggcaagagcacacccagtagaggggagagtaatagcaaaggctcagggcaggaagggcaagggagaggccagtgggtgaggtcacatgtgaagggcatacaatgggcaaagacaaggccagagtggccaggcccaatcctccaggacttgcagacctgggaaagagtgcatctccatcctgggagcagcaggaaaccactcaggcctttagaagatccttctggcagctgtgtagagaatgggtggtgtgatccttccatgcatgggctcatgtacgtgattaccaataactgtcgagtgacagtgtgaggagggctgcaagccatgagtgtaggcacagcagacagactcacctttgtctggcggtgagatggggtgggaagtgtgccaagttgacctcccaaagaaatgatattttagtggaagaatgaatagaatcagagaagcaaagtaagagggaagagcagagaggacagcagggacaaggacttgggggcaggaagaggaaaggcaggttaaggacatgaaagatggccaggctggctggagctcaggcccagcaaggccccctgggggccatggtcatgggtgagcttgggtttggcttctgttttcgtcttgggcttctgtgaaagcctcgagcccttgcggggaaccagtgaagctgtgtgtgcatcttctgtggggagtgccagagtcttcagggagcactccatcttctctcctccccacaggctgctgtggctgcatcagggctcaacacgatgcttgaaggtaacggccagtacacgcttttggccccgaccaatgaggccttcgagaagatccctagtgagactttgaaccgtatcctgggcgacccagaagccctgagaggtgagcatcctttggctcctgctgctgcctcatttgtgcagctagattgagcccaagacctgctctggtccaagatgaacataccacctgccatgaggtgaccctcaggatatccactgcagccatgggctggggtcatcctgtcctgttgcttcagctaaccgtgtctctagcagccacactactctgagggctgactacagaatccagcagcttttgtctgggagagctggactgaagagaggcatagctggagacccatagctggccctggccagaaacagggagagtgaaaggctggaatagccaaggccagagcaaggctaataggtagagcaacagcttacaggtgtgggggtggcagatactggcacccttgaaatggattcctcatgcccacgcttcactattcttctctgtggctaggggatttatggataaaccaaaattacagttaaaaaccagccataggccaggcacagtgactcacgcctttaatatcagcactttgggaggacaaggtgggcggatcacctgagatctggaatttgagaccagcctggccaacatggcgaaaccccatctctactaaaaatacaaaaattagctgggcatggtggtgggcacctgtaatcccagttactcaggggctgaggcaggagaaccacttgaacccaggaggtggaggttgcagtgagccaagcttgcaccactgcactccagcctgggtgacacagcgacactccgtctcaagaaaaaaaaaaaaaaaaacagttatagtagtcaacttttgactctccatttcagatttcgtcatgccctcctcaatgagctgctaagttaggcagtgcattgattattgctgcaggagagggaaggaaggagctaacgtgttttcacatgttttccttttggagatgagaaaggaggactctgccttccccctaccctgcccctttctactccaggacctctgaaaggccatgagcacaaagctgctgcctgagtcccctgaaatgcagggtacgccccaggtctctgatgtaccccaccacacttttcctctcaaacatattccaggatcacttgatttcttttgaatctatttaaacccaccgtgtcaatgtgctatataaaatgtctaatgcatttcagacaccctatacatctatacatttaaagtgttctccttctatctgtgcagggatgggaaagggcatatttctgaaagcacagatgggaagacgggatttgttccgtgtccaggtgattatggtacctctatgcgcctggccggcactggggacagaggccatgaaaatgaatacagcacagcctttgcctccaagaaacttaagacctagtagaaatggcaggctttaaaacaggttgttgggatctgatttggtgagtgcaatgacagagatactcacagcacaaaatggggaatgagggcgggcattgggacacacatagccttaaggggcccaaaggcttttagaactgtattccctattaaaacatgatttgcacagagcacattctttgctttggagacctcagaactccttactataggccgggcatggttataatcccagcactttgggaagccaaggcgggcagatcacttgaggctgagagttcaagaccagcctggccaacatggtaaaaccccgtctctactaaaaatacaaaaattagctgggtgtggtggtggccacctgtaatcccagctactcaggaggctgaggtaggagaatcacttgaacctgggaggcagaagttgcaataagcccagatcatgccactgcactccagcctgggcaacaaagctagactctctcaaaagaaaaaaacaaaacaaaacaaaacaaaacaaaaaaaactccttattataaactgtaagaaaaaaaaggcccctacttcgtcccttttgcaaatctgccttttcctactcactaaccagctggttcagagcaaggacactctgtttggtgccatcgctgcagactggaaggaagaggtccttgccccacacccaacagtctcctgctgttaccggcaggttggcaggcaggcaggcgagaagcagccagggctggtggtgtgtccagtttgaagactagtttccagccctggccctgctcaccctccaagtggccctggcaggttcctctaccacatcgtggacttcaccttccttctctaagaagctcaatccccaaggcctcattcccataggccttctcaccctttttctttccctctggctgaatgtggccagcacgggcttccaaggccatcaactcgtctgcagcagccccatgccttgcagggcctcagagcttcctcctgcctatgacagtgtggttttggttcccacacttgggatcagattgaaactcgcctccgtggtgagaatatgggacatagagcctcggtgaccttggtgagcagcagtccaggccacctgctcagcctggggttgggggggggctcctcctccttgactggtccttgcatttgcctccatccagcctgtctgggctctccgaggcaatggagaccagcaggagtcacgatgggtcaggagccccctttgggcctcagccctgccctgccccctaaagtagcacttggataagcaaataaattattatacttactatttatgggtgtggtgaatgggatggcaaaggccaagtcttactgatcaccaaaccttaagatatatcctggcagctagtagacccttgggctaaatgaacagaaaactggacaaataaagtgtacacaaataactcaaagctgtcatttgtacacttttcgtcttttcctactacagtttacatttttataaaggtgagtagatttctaaaatcccgtggtaggctctcttgagtttttcttgtatccctgaagttcagctacaaataagctaatcactaacatttgttgagcatttactctgttgtcaggccccgtgccgagtgctttaggttcagaatttcatgtcatccccacagcagccctaggagatgaatgcaattcttatgtccacttgactgataaggaagttgaggttcaaagaggctaaatgactctcccagggtcccacagctggaaagtggccacagggccccagctggttttctagggcagcaggcagaaggcgaggaggatctgggccctgtggtgccccagcctcatctgagggtcctcatctgagagaacaggatcctcacagcatgggcaggctgcaagtggtccctgaggttatcgtggagtggaccctgacttgacctgagtctgtttggaccccagacctgctgaacaaccacatcttgaagtcagctatgtgtgctgaagccatcgttgcggggctgtctgtagagaccctggagggcacgacactggaggtgggctgcagcggggacatgctcactatcaacgggaaggcgatcatctccaataaagacatcctagccaccaacggggtgatccactacattgatgagctactcatcccagactcaggtaggccaggcctccgggggccttggccctgcctggcccaccatctcttctgccatcctttgtggcgggggaggggaaattcagagatctttgggcgacttccctgcctggacccagctcacagcttctcggccactgcaaatgtgtgggttgtgaccagactgatgtgtcttgagcttcaggcttgcaagtgcagtggagaggcagtggggagctattgaaggggtctggggacagactcaatcacagaggcctttcagaagatctgcctgctgtgcatgggcaaagagggccacttgctgacctcagagcatgtgctttctcagtagtgcccaagctgtcccatggtcactgacccagttagaatgactgaatggactttggcttgtgtctcattaggaatcctagccccattctagtcttccagtgagatctgtccatgagtgaaggaatctcacaggaaaaaacaaaatgcttctatgggtgtggttgctggccttatctacaccacagaagccatcacacagactgtctttcttcccattgttagaatgtgccctgaccaagcagcccacagggcctgggacagaggctgatctctgcctaactgagctcacctctcctccctctcctcctgactggttagattttctaggtgactgttcccctgatgacacaagcccgctgggccccagcagtgtttagaggggttgttgactcacgagatgacattcctgctgatgtgtgtcatgccctggggtggatgaatgataaatgaaaacagcgcttttaacttttgaacccactttctccttccttgtagccaagacactatttgaattggctgcagagtctgatgtgtccacagccattgaccttttcagacaagccggcctcggcaatcatctctctggaagtgagcggttgaccctcctggctcccctgaattctgtattcaaaggtaacatggggaaggcatccctgttagattgtccctggaggcagcttccccacccctgtcacctccacaacactctccgatttacagcaccccatgggacattagaacttccactcagctcaaccaaaagcagatgtgacttcagcagaaacttcagaggctctgttgtttcattaggcagtgcagagaatgcctttggggagccgttcctcagaactcaagacttgacatctgggaggcagccgttcctcagaactcaagacttgacatctgggagagcagagcattcccttgcctttctatttgcagggtcacttgccaatgtatagtcaagaggtcagagtgagggtacagctgagctgcagccccaggaaggcagagaagggggccaagttgtgtgcgtgcctgcccttccctcttagggcaaaactccaaacacccttgattatctggatcttctttaattctccatagaagataccagatgttaaggaatattggcagcttcacttggtttctcaatccctgtttccaaactcaaggagggatgggctttttcactgtatttatctctcatcactctcttcattgcaggagcacatctctctggacctaaccatcaccctttcttgtagatggaacccctccaattgatgcccatacaaggaatttgcttcggaaccacataattaaagaccagctggcctctaagtatctgtaccatggacagaccctggaaactctgggcggcaaaaaactgagagtttttgtttatcgtaatgtaagttctgggtcctaaatcatgctcctgggaagctccttactgtgggacttgtattagtgtaaaaaaaaatgtcctcaataagcaggagtttgcatgagaactggttgctgacaaggaaggaaataatttctggaaaatatagataacaaaatgagatcctgcagaaggattggaatctctttttctggaggcctttgagaataaaccacacaattatccaacctgtattgtgaaggaataagtccttcttgaattcaggaattaacacctgggaggagggatggagttcagactctttctgagcttatgagaagagaagccccctaaactaaaatacagccctccttggtccaaaaggtgccttctctcttctgctgtatcttctttgttttcaaacccaacagttaccctggaaatcaaaaaggaagtacaactcaacatagctcttgcctgggaccaaccagcaccatttggctaaagatggttatcatctgttaaacaaagaaataaataaatgggttcaacgtatttatttcaacattgtcaatggacctcatgtgtaactgatattctcattatgggacctctgtgtgactttattggggcctctctaaccgttctttccttaaggaagaccatttattgttttatttcctggagaaaatacatcattttatcccagccttaataacccatcccagtgtatactccttcatcttcatggataatgaccctgctacatgctctgaacaaatcaggaggcccctcgtggaagtataaccagtcctttctttctctgtccctcttctgtgcagagcctctgcattgagaacagctgcatcgcggcccacgacaagagggggaggtacgggaccctgttcacgatggaccgggtgctgacccccccaatggggactgtcatggatgtcctgaagggagacaatcgctttaggtaattagttccatccccgggtggagcttctgcccagtggtcatgctggagtgggatgtggggccccagctatttgtcaagctttcttctaccttggggattcaattaacactagcagtgcactgctgcgaccttccagacttgggatggggaaaaggcaagggtcgccttgaaagcttacattgggaagaagggttacttctaagagtgtaatcttcacatgcatgggaagcagggaggggggactacatttttatgactgaagtgcaaggaaaacatcaccctctcattgtaaagctccaagtgagccaagagcacatagtttacagtgcacgatgagcctctcactctctgcgcagtatctgtttattgcaactgaagcacccttgtgagtttgttttcttgcccggctatctccatttctgacttgctcattcaccttggggtgctgtcatattgaatgtttccctgtcactgacttcagccacctgcacaagggcttggagaccacacccctctgccctcccagaatcatatccctggaggctcagctagtctctgggtcagccatacctctgccctttcttttccctcctttctcctgtggcctctgacgtctggccatttaacagagcttagcatttttgctgggtggagagagctggagcctggaatcactccctctttgtgcatacggagggcatgaaaaccaaggtgtgtgcattccagtggcctggactctactatcctcagtggtgaggtatttaaggaaaatacctctcagcgtggtgaggtatttaaggaaaatacctgttgacaggtgacattttctgtgtgtgtatctacagcatgctggtagctgccatccagtctgcaggactgacggagaccctcaaccgggaaggagtctacacagtctttgctcccacaaatgaagccttccgagccctgccaccaagagaacggagcagactcttgggtaaagaccaacttaagtacacgtctccatttttctaaagtagtgatccctcagggccccagcagcaaacagttggcacatcaaggattgacttgaagggattttatgacaagactattagtgaaagagtgggcgggactaaaggaactagcaaaggatgaggccaaccagggactagcaaccctgggaagcctttactacccctaggcctgggggaatgggaggatgagagcaggaaccagggaggtcatgagccttggacaagggcacagaacagcagccagagccatgtgcagccagccactgtcagaaccatgcaagggggaccactcagcgccccagcctccctctcagacagttgccatctgggtctcttgttggctgatgcgagagcaggagggagcccactgatgcagttcatagagctcagcctcctgggcaggaaaccgggcagagaggagtagaaaagaattaagggtggctgcgaccagcccagtcactgaggcacgtttcccactggagacctatgagcacagtgataataaagccagttacctgcactgactatccctccagacaaaagctttcccaagaagttagtcatggctctgagagatctagttgaggatgtttggcaggggatctagtggttacgggtggctaagaaaaatgaggaaggtaagagtatcttgcagcctgtgttgggaggattaaataggatgccacacacagggccaggcagacagcctggtcagtaatagccatgacgatgggggcggggggagcaggaatgggagttgcagtgtttagctcagatgcatgcctgtgagagatgcttccactctcacagaaagatgagaccaaggaaaaggaggaggaagaggaaggaccttgacaaaccttggggcccacattgtctacacctcccttcctgctctagagcagaatagaaagttcaggttgcaggcagctctaagttgaattcgtgtcctgtttaattttctttattgctaaatgaatgcctgtgtctgtgatgctgacgtatgttcctaaggagaggggagaagttcattctgaacataaacttttcatcctctctctgtccagcaagaatggaatattccccaagtggcctgagccagcttggctttctttttgttttcaattatgtgggagttgaggagggggatgggaaaagcttcccaaacacaccctcccccaggcctgaggcacccctgggggacagagagtgttagaggttggtacaggcgttagagatattgaaaggacatcccatgcaccccaggggctggtgtggctctgtacttccaggcaatattttgtggaaggggaaccttgtcagctccaggttgtggatgtttgaaaatcagttggtacccagtggctccatcctctggcaggcatgtggatttgtcaataaccaagtgaactctccaaaataagttaaaacttcctcccttctcagtttcaagatgctggaaatagctgttcataagccctggggaaatttagccctttggctggtaatgggagtatccgagatgagagggcagctggaaactttcggaatgacctcccacacttaatttgggaaatgcctctgcacctttatgggcaaccagatgcctgccccagttgctggagacactgatgtgggctgaaaggaatgctgagacgtgacgaggagagatgctgcggagggaatatccccctcagccctgacctcatcggctccatggctcctccacagtacagctgtctactcttttaagttctcccttcaggaaatagccatctcaaacagaatgtgcatttgagggcagaatgtgtaaatattgcactactgtgttataaccgtcaggagccatgctgatgatgaaacgtcccagatgccggtgctggaaaggtccctggctttccaagcaaatatttatctcatggaaacatgagtcatactcacagaggagtatggattaactccttctcagcagccagggagcccagcatcccagacagcatatttaacccagaggccaactgactgctggggcagatttgtggtcatgaacatgtgctttgtgtcctctgaccattagacagattgtgggtcacaacgttgagtatacagtgggagcttaataagtgcttattccctgggcagggagttcttcatttcaggggtgaccacttacatcttctcctctgggccctccttgaccaggctaattaccattcttgggattaactctatctccttttcccgcaacctgcaggagatgccaaggaacttgccaacatcctgaaataccacattggtgatgaaatcctggttagcggaggcatcggggccctggtgcggctaaagtctctccaaggtgacaagctggaagtcagcttggtaagtgtcctgcaaatcaaaggctggctaaatttccccagggcagggctccaggacatatctcacccccaggatggaattatacacacacaaccttcaagttgcagcccgaatctctgagtgtaattcgtccaaagaaaaagagaaaagagaagagggtcttcagggaaatcaagtgagatcatagttagacatgagtaagaacttccagatttacaagggaatagagcatctgatttggcatctgagagaggctattagatcttccttctcttaaggaggttgtaggcaactagttatgtgactgaagagatcagtctgtactcacaccatcccaccccccaaacccagggcttcactgagttgtaccatgaaccagaccatcccaagaggctttttgagttctgacacttgctctgtgagccttcccttgctctgcacattgatgatataactttgtaactgcactaagagtgttcctaaagcagatagccagccgagctccagaaatctccctggctgcacctgcagaggccactgacccctctgtggagggaccgctcttcagtgtgtggctggcttctactctctgctcctctctcttggtcttcagccatccattgctcaccagtttctcaccaggagcataggaagatatgcatgtagggaggtaggcacggggatgacttgtttgactttaagcaggtcattcaagaatctcctcgcacctggtttcagatgctggggtcctgtctgtcacaggcttctgtgcctcctacccccttgagtttgtcacatggcccttcaggaaggcctgagatagatttgccctgggtgggcctcctatgagaaaatcttaagtgaggcacccaggcaaaatggaaagagccttttgcccagagcaggaagcctgtcttccatttccagctgttccacctacttagcttaaaagaggcacttcgcctgtcttcagtctcagtctcagtctcctcttctgtggaatgggacaataatatctactctccttatcatacactgctgtgaggactgagtggatcacacaaaaaagcattatgtaaattgcaaagtgctaaatccacacaggagatttgaattaatccaccacactgaaggtctgtcaagggcagggactgtttcattcaccagagtatccccagtcttacacaggacttggcatatgaaaagtgttcagtaggccgggtgcagtggctcatgcctgtaatcccagcactttgggaggccaaagtgggcggatcatctgaggtcaggagttcaagtccagcctggccaacgtggtgaaacctcatctctactaaaaatacaaaattagctgggcgtggtggcacatgcctgtaatcacagctactctggaggctgaggcaggagaatcacttgaacccaggaggcggaggttgcagtgagtcgagatcatgccactgcactccagcctgggcgacaagattgaaactccatctcaaaaacaaagaacaaggaaaaaaacgaaaactgttcagtaaacacttgctgagtgaataaaataaataaataaatgtataaataaatgctctactttcaaccactactctgtttttcttttagaaaaacaatgtggtgagtgtcaacaaggagcctgttgccgagcctgacatcatggccacaaatggcgtggtccatgtcatcaccaatgttctgcagcctccaggtaagtgtcgcatccccactgactctgcagccagtccttttctccatgtggcagttggtggagagaagaaaaactgttctaaacaatgatgagaataacatgtaattgtgatagttaaactgtgcctatgtgactgattgcagagtgaattgggagctgttggttttgaatgcaccacactaaggaatgtgaggacacattgctctttgcggagttgcccagctatattagctcccctcggacacagcccagttttctgtattcgcgtggatgctgtccgcgcgattcccagcactcctcttacagcatctcacctcagtgtatgttccttgcctccagtgcagttgaacctcagtcctgcctctcctcatgtgtgcattcacctttcttggcgctctctccccatgggccaagttctaccatgagttatgaaacattatggagaaaacatgtctttggaaatgtgagccagaaagcccaccagtgcccctcagtcacggttgttatgaatgacatgctaatggtttcactctggtcaaacctgccttttctttcctcttcagccaacagacctcaggaaagaggggatgaacttgcagactctgcgcttgagatcttcaaacaagcatcagcgttttccagggtaagatgcctgctaggtttgcgcctagcctgagcagcctcaggtcctctgtttgggccatagaggagcctctccagcccctgtcttccttggctgctccccagggctctcttaaaacttctccccactcccactgaggcatcctcagccccagcctgtgtcaaattcagagtaaagaaccaaggcaactccctggctttcatgggccaaagcgcaggctttcacaccgaggcctctgagcctcagatcatggggaagtcactgctggagagaacagacatagctctggaagccatctgcccaagagggcagcccatcccaagttcatcttacagtggccaggcctgccctgagccggggcctctgggtcactcttctgctgtccatggcattgcccatcctgggtgaggctggggctctcctgggcactgtatgtattctggatacagggatactgggctcgctatgtgtgtggagccatcccttccttgccccagccccacctccctctcaaaccctctctggctctttctgagcttcctttcctgctccccagcttgcccagtgctcagtgccccacttggctcttttgctacttcgggtcaggtggagcctcttgggaatgtgaagtgccttacagaaagattgcacttcaagaggagaggctgcagggagccatcctaaacccagaggcctggagcttactgtgtcactttacttttgtacacaggggtctccttagtgccctcgagaaggattcttggccctgagcttctactcctgaggccacctctgtgcagccccagctccctcaactctaggctgtagtctcagtgggaaagcctggcttgggggtctcctaggaatgtccacctgaaggcacacttgataggggcttgcacaacttatgtctgccaaggccacctgaggaactccctggtgcctataagttccaccttccccttcctcttcctcgccccagcattttttctgagtaggggtggcaatgggcaaagccattgtcataagcagttgcaggtataactttcactagaaaacctgacaccttgtgttttctttcaggcttcccagaggtctgtgcgactaggtgagtctggtctgggtttgaagtcattgcagacctgtttaggccttacccccaagcaagcccaagcctgccatctgctgtatatagataagaacatcatggtgcagtaaaagaagcctggcctttggagtcagaacagcagggtgacttggggtcagacccagagcaccccatttccttctctgtaagatgaggataataagagtaacaaccttttagggttaaggtgagttttcagcttaggaagtctgggaatattgcaaagggcttggcaggaacccatggtgaggatctagttccaagttgataggtacagaaaaccagaacatcgggccttgagtaaagagtgaagtttcacaaaccacaaagcacctgctatgtgcaggagagcatggcagaaggaggctgcttggccctggtccttgagattctgacagtgtcctagacagacatggggagatctgcacctatttgacgttaccaacttctctttttcagcccctgtctatcaaaagttattagagaggatgaagcattagcttgaagcactacaggaggaatgcaccacggcagctctccgccaatttctctcagatttccacagagactgtttgaatgttttcaaaaccaagtatcacactttaatgtacatgggccgcaccataatgagatgtgagccttgtgcatgtgggggaggagggagagagatgtactttttaaatcatgttccccctaaacatggctgttaacccactgcatgcagaaacttggatgtcactgcctgacattcacttccagagaggacctatcccaaatgtggaattgactgcctatgccaagtccctggaaaaggagcttcagtattgtggggctcataaaacatgaatcaagcaatccagcctcatgggaagtcctggcacagtttttgtaaagcccttgcacagctggagaaatggcatcattataagctatgagttgaaatgttctgtcaaatgtgtctcacatctacacgtggcttggaggcttttatggggccctgtccaggtagaaaagaaatggtatgtagagcttagatttccctattgtgacagagccatggtgtgtttgtaataataaaaccaaagaaacatacgtcctgtgtgcatggtacagtgtgctgacctgaggccgtcatgctcctccacacctcaattctgctctggagaagctcagaaaggagccccgagggatggttttggggagattccagcagccagccctcagacagccagacagctcatgggggtttgagcctgtctttgccaaacaggtttttatttcaccctcctccggtcctggggtttcaagttttcagtgttgccttcaccccgcactttattcctcttattacttggaagtaccttccctccagcatggtgatcccctgcctgtgtgctggacttttgagtcctcagcaccaacctgtgaagtggttgccagcataatcccattatgcagatgaggagaccaaggcccagggaagggagaaccaccagcagcacgtaaaatagctgagctgggactggaactcacacctcctgactctcagtgaccaccactgacaacagcataagtccaggttttccaggcccatcccctctgtgccaacccacattcagattccttccccggctcccgtaatctctggcatctagaatatcctcaggactctgagaggtgatatcatgtggttgtggtgccattgccccctacctgtgtggcctggggccagtcatgtgacctcccagggtctcctcttctgtaatagggagatgaccgtcacatctacttcatgggtccatcgtgaggatgaaatgagatgatctatataaaatgcttggtacaacattaggtggccttatttttatcctgccgtctgggactgctcaggatcaatgcgccagagagcctttatttgtgtctttcccacaggtgggctggcccactttcctagagaatgggacagacctccttcccacccacacccatctctgccaaggctgattcactccagcaggcggagctcatttcacttcatggaaccaatgacccaaagatatatccccagcactactgctggtcagtccactgctgctgggaatacagcaatggtagtggcagacagaggccctctcttaaatagcttccagtctgaggaaagagagatatgacatcaatccattaaaatcattcatccattggttccacaaatatt

SEQ ID NO: 77 - protein, 683 aa, Homo sapiens
MALFVRLLALALALALGPAATLAGPAKSPYQLVLQHSRLRGRQHGPNVCAVQKVIGTNRKYFTNCKQWYQRKICGKSTVISYECCPGYEKVPGEKGCPAALPLSNLYETLGVVGSTTTQLYTDRTEKLRPEMEGPGSFTIFAPSNEAWASLPAEVLDSLVSNVNIELLNALRYHMVGRRVLTDELKHGMTLTSMYQNSNIQIHHYPNGIVTVNCARLLKADHHATNGVVHLIDKVISTITNNIQQIIEIEDTFETLRAAVAASGLNTMLEGNGQYTLLAPTNEAFEKIPSETLNRILGDPEALRDLLNNHILKSAMCAEAIVAGLSVETLEGTTLEVGCSGDMLTINGKAIISNKDILATNGVIHYIDELLIPDSAKTLFELAAESDVSTAIDLFRQAGLGNHLSGSERLTLLAPLNSVFKDGTPPIDAHTRNLLRNHIIKDQLASKYLYHGQTLETLGGKKLRVFVYRNSLCIENSCIAAHDKRGRYGTLFTMDRVLTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPANRPQERGDELADSALEIFKQASAFSRASQRSVRLAPVYQKLLERMKH

SEQ ID NO: 78 - DNA, 660 bp, Homo sapiens
aaaaaacagcccggagcctgcagcccagccccacccagacccatggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctagttgcctcccatctgcagagcttcctggaggtgtcgtaccgcgttctacgccaccttgcccagccctgagcc

SEQ ID NO: 79 - protein, 204 aa, Homo sapiens
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 80 - DNA, 2475 bp, Homo sapiens
agccgctctccgcatcccaggacagcggtgcggccctcggccggggcgcccactccgcagcacccagcgagcgagcgagcgagcgagggcggccgacgcgcccggccgggacccagctgcccgtatgaccgcgccgggcgccgccgggcgctgccctcccacgacatggctgggctccctgctgttgttggtctgtctcctggcgagcaggagtatcaccgaggaggtgtcggagtactgtagccacatgattgggagtggacacctgcagtctctgcagcggctgattgacagtcagatggagacctcgtgccaaattacatttgagtttgtagaccaggaacagttgaaagatccagtgtgctaccttaagaaggcatttctcctggtacaagacataatggaggacaccatgcgcttcagagataacacccccaatgccatcgccattgtgcagctgcaggaactctctttgaggctgaagagctgcttcaccaaggattatgaagagcatgacaaggcctgcgtccgaactttctatgagacacctctccagttgctggagaaggtcaagaatgtctttaatgaaacaaagaatctccttgacaaggactggaatattttcagcaagaactgcaacaacagctttgctgaatgctccagccaagatgtggtgaccaagcctgattgcaactgcctgtaccccaaagccatccctagcagtgacccggcctctgtctcccctcatcagcccctcgccccctccatggcccctgtggctggcttgacctgggaggactctgagggaactgagggcagctccctcttgcctggtgagcagcccctgcacacagtggatccaggcagtgccaagcagcggccacccaggagcacctgccagagctttgagccgccagagaccccagttgtcaaggacagcaccatcggtggctcaccacagcctcgcccctctgtcggggccttcaaccccgggatggaggatattcttgactctgcaatgggcactaattgggtcccagaagaagcctctggagaggccagtgagattcccgtaccccaagggacagagctttccccctccaggccaggagggggcagcatgcagacagagcccgccagacccagcaacttcctctcagcatcttctccactccctgcatcagcaaagggccaacagccggcagatgtaactggtaccgccttgcccagggtgggccccgtgaggcccactggccaggactggaatcacaccccccagaagacagaccatccatctgccctgctcagagaccccccggagccaggctctcccaggatctcatcaccgcgcccccagggcctcagcaacccctccaccctctctgctcagccacagctttccagaagccactcctcgggcagcgtgctgccccttggggagctggagggcaggaggagcaccagggatcggaggagccccgcagagccagaaggaggaccagcaagtgaaggggcagccaggcccctgccccgttttaactccgttcctttgactgacacacatgagaggcagtccgagggatcctccagcccgcagctccaggagtctgtcttccacctgctggtgcccagtgtcatcctggtcttgctggccgtcggaggcctcttgttctacaggtggaggcggcggagccatcaagagcctcagagagcggattctcccttggagcaaccagagggcagccccctcactcaggatgacagacaggtggaactgccagtgtagagggaattctaagacccctcaccatcctggacactctcgtttgtcaatgtccctctgaaaatgtgacgcccagccccggacacagtactccagatgttgtctgaccagctcagagagagtacagtgggactgttaccttccttgatatggacagtattcttctatttgtgcagattaagattgcattagtttttttcttaacaactgcatcatactgttgtcatatgttgagcctgtggtctataaaacccctagttccatttcccataaacttctgtcaagccagaccatctctaccctgtacttggacaacttaacttttttaaccaaagtgcagtttatgttcacctttgttaaagccaccttgtggtttctgcccatcacctgaacctactgaagttgtgtgaaatcctaattctgtcatctccgtagccctcccagttgtgcctcctgcacattgatgagtgcctgctgttgtctttgcccatgttgttgatgtagctgtgaccctattgttcctcacccctgccccccgccaaccccagctggcccacctcttccccctcccacccaagcccacagccagcccatcaggaagccttcctggcttctccacaaccttctgactgtcttttcagtcatgccccctgctcttttgtatttggctaatagtatatcaatttgcactt

SEQ ID NO: 81 - protein, 553 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSPRPQGLSNPSTLSAQPQLSRSHSSGSVLPLGELEGRRSTRDRRSPAEPEGGPASEGAARPLPRFNSVPLTDTHERQSEGSSSPQLQESVFHLLVPSVILVLLAVGGLLFYRWRRRSHQEPQRADSPLEQPEGSPLTQDDRQVELPV

SEQ ID NO: 82 - DNA, 505 bp, Homo sapiens
aaagttctctggaggatgtggctgcagagcctgctgctcttgggcactgtggcctgcagcatctctgcacccgcccgctcgcccagccccagcacacagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaaacagcactgccctccaaccccggaaacttcctgtgcaacccagattatcacctttgaaagtttcaaagagaacctgaaggactttctgcttgtcatcccctttgactgctgggagccagtccaggagtgagaccggccagatgaggctggccaagccggggagctgctctctcatgaaacaagag

SEQ ID NO: 83 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 84 - DNA, 1080 bp, Homo sapiens
ccggggggcatgagggtccgagacttgttcttctgtcccttccaagacccggcgacaggaggcatgaggggcccccggccgaaatgacagtgctggcgccagcctggagcccaacaacctatctcctcctgctgctgctgctgagctcgggactcagtgggacccaggactgctccttccaacacagccccatctcctccgacttcgctgtcaaaatccgtgagctgtctgactacctgcttcaagattacccagtcaccgtggcctccaacctgcaggacgaggagctctgcgggggcctctggcggctggtcctggcacagcgctggatggagcggctcaagactgtcgctgggtccaagatgcaaggcttgctggagcgcgtgaacacggagatacactttgtcaccaaatgtgcctttcagcccccccccagctgtcttcgcttcgtccagaccaacatctcccgcctcctgcaggagacctccgagcagctggtggcgctgaagccctggatcactcgccagaacttctcccggtgcctggagctgcagtgtcagcccgactcctcaaccctgccacccccatggagtccccggcccctggaggccacagccccgacagccccgcagccccctctgctcctcctactgctgctgcccgtgggcctcctgctgctggccgctgcctggtgcctgcactggcagaggacgcggcggaggacaccccgccctggggagcaggtgccccccgtccccagtccccaggacctgctgcttgtggagcactgacctggccaaggcctcatcctgcggagccttaaacaacgcagtgagacagacatctatcatcccattttacaggggaggatactgaggcacacagaggggagtcaccagccagaggatgtatagcctggacacagaggaagttggctagaggccggtcccttccttgggcccctctcattccctccccagaatggaggcaacgccagaatccagcaccggccccatttacccaactctgaacaaagcccttgcccccatgaaattgtttataaatcatccttttctccca

SEQ ID NO: 85 - protein, 235 aa, Homo sapiens
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 86 - DNA, 1321 bp, Homo sapiens
ccgcctcgcgccgagactagaagcgctgcgggaagcagggacagtggagagggcgctgcgctcgggctacccaatgcgtggactatctgccgccgctgttcgtgcaatatgctggagctccagaacagctaaacggagtcgccacaccactgtttgtgctggatcgcagcgctgcctttccttatgaagaagacacaaacttggattctcacttgcatttatcttcagctgctcctatttaatcctctcgtcaaaactgaagggatctgcaggaatcgtgtgactaataatgtaaaagacgtcactaaattggtggcaaatcttccaaaagactacatgataaccctcaaatatgtccccgggatggatgttttgccaagtcattgttggataagcgagatggtagtacaattgtcagacagcttgactgatcttctggacaagttttcaaatatttctgaaggcttgagtaattattccatcatagacaaacttgtgaatatagtcgatgaccttgtggagtgcgtcaaagaaaactcatctaaggatctaaaaaaatcattcaagagcccagaacccaggctctttactcctgaagaattctttagaatttttaatagatccattgatgccttcaaggactttgtagtggcatctgaaactagtgattgtgtggtttcttcaacattaagtcctgagaaagggaaggccaaaaatccccctggagactccagcctacactgggcagccatggcattgccagcattgttttctcttataattggctttgcttttggagccttatactggaagaagagacagccaagtcttacaagggcagttgaaaatatacaaattaatgaagaggataatgagataagtatgttgcaagagaaagagagagagtttcaagaagtgtaaattgtggcttgtatcaacactgttactttcgtacattggctggtaacagttcatgtttgcttcataaatgaagcagctttaaacaaattcatattctgtctggagtgacagaccacatctttatctgttcttgctacccatgactttatatggatgattcagaaattggaacagaatgttttactgtgaaactggcactgaattaatcatctataaagaagaacttgcatggagcaggactctattttaaggactgcgggacttgggtctcatttagaacttgcagctgatgttggaagagaaagcacgtgtctcagactgcatgtaccatttgcatggctccagaaatgtctaaatgctgaaaaaacacctagctttattcttcagatacaaactgcag

SEQ ID NO: 87 - protein, 245 aa, Homo sapiens
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKGKAKNPPGDSSLHWAAMALPALFSLIIGFAFGALYWKKRQPSLTRAVENIQINEEDNEISMLQEKEREFQEV